SEQUENCE LISTING (34 sequences)

SEQ ID NO: 1 - DNA, 34 bp, D156A
cattgccttcttgattgcgggctctggtagcatc

SEQ ID NO: 2 - DNA, 34 bp, V254A
gcctttaagatcctagcggtcatcacggatggag

SEQ ID NO: 3 - DNA, 34 bp, Q327A
gaagaccattcagaacgcgcttcgggagaagatc

SEQ ID NO: 4 - DNA, 32 bp, I332A
cagcttcgggagaaggcgtttgcgatcgaggg

SEQ ID NO: 5 - DNA, 32 bp, F333A
cttcgggagaagatcgcggcgatcgagggtac

SEQ ID NO: 6 - DNA, 33 bp, E336A
gaagatctttgcgatcgcgggtactcagacagg

SEQ ID NO: 7 - DNA, 30 bp, Primer
attggatccgctggcaccgagattgccatc

SEQ ID NO: 8 - DNA, 30 bp, Primer
aatttctcgaggtctccaaccgtgccttcc

SEQ ID NO: 9 - protein, 27 aa, Amino acid insertion
PKGRHRGVTVVRSHHGVLICIQVLVRR

SEQ ID NO: 10 - DNA, 20 bp, primer Eo26-H3
gaggggaagcttagtgggcc

SEQ ID NO: 11 - DNA, 19 bp, primer Eo-24
gaagttggcctgagcctgg

SEQ ID NO: 12 - DNA, 25 bp, E-cad 5′#1
ctgcctcgctcgggctccccggcca

SEQ ID NO: 13 - DNA, 27 bp, E-cad 3′#1
ctgcacatggtctgggccgcctctctc

SEQ ID NO: 14 - DNA, 45 bp, primer Ecad5′Kozak
gcgttaaagcttcacagctcatcaccatgggcccttggagccgca

SEQ ID NO: 15 - DNA, 33 bp, Primer Ecad3′(Xho)
aggcgctcgagaatccccagaatggcaggaatt

SEQ ID NO: 16 - DNA, 26 bp, primer MAdCAM-1 5′#1
atggatttcggactggccctcctgct

SEQ ID NO: 17 - DNA, 24 bp, primer MAdCAM-1 3′#5
ctccaagccaggcagcctcatcgt

SEQ ID NO: 18 - DNA, 49 bp, primer Mad5′Kozak
gcgttaaagcttcacagctcatcaccatggatttcggactggccctcct

SEQ ID NO: 19 - DNA, 57 bp, Primer Mad 3′ #6 Sal
gctagtcgacggggatggcctggcggtggctgagctccaagcaggcagcctcatcgt

SEQ ID NO: 20 - DNA, 28 bp, Primer Alpha1.5
gactttcagcggcccggtggaagacatg

SEQ ID NO: 21 - DNA, 28 bp, Primer Alpha1.3
ccagttgagtgctgcattcttgtacagg

SEQ ID NO: 22 - DNA, 29 bp, A1.5Nde
atatcatatggacatagtcatagtgctgg

SEQ ID NO: 23 - DNA, 33 bp, A1.3Bam
atatggatccctaagacatttccatttcaaatg

SEQ ID NO: 24 - DNA, 29 bp, A2.5Nde
atatcatatggatgttgtggttgtgtgtg

SEQ ID NO: 25 - DNA, 32 bp, A2.3Bam
atatggatccctatgacatttccatctgaaag

SEQ ID NO: 26 - DNA, 28 bp, A1.I.Bam
cggatcccccacatttcaagtcgtgaat

SEQ ID NO: 27 - DNA, 31 bp, A1.I.Pst
gctgcagtcatattctttctcccagagtttt

SEQ ID NO: 28 - DNA, 22 bp, Primer specific for 5′EcHPPK
gtagatgacagtggcgtatatt

SEQ ID NO: 29 - DNA, 23 bp, Primer specific for 3′EcHPPK
gccttaccatttgtttaatttgt

SEQ ID NO: 30 - protein, 159 aa, amino acid sequence of E. coli HPPK
MTVAYIAIGSNLASPLEQVNAALKALGDIPESHILTVSSFYRTPPLGPQDQPDYLNAAVALETSLAPEELLNHTQRIELQQGRVRKAERWGPRTLDLDIMLFGNEVINTERLTVPHYDMKNRGFMLWPLFEIAPELVFPDGEMLRQILHTRAFDKLNKW

SEQ ID NO: 31 - protein, 10 aa, Histidine tag
MGHHHHHHGG

SEQ ID NO: 32 - DNA, 52 bp, 5′EcHisHPPK
cgccatgggccaccaccaccaccaccacggcggcatgacagtggcgtatatt

SEQ ID NO: 33 - DNA, 29 bp, 3′EcXhoHPPK
cggctcgagttaccatttgtttaatttgt

SEQ ID NO: 34 - protein, 169 aa, amino acid sequence of His(6)-HPPK gene
MGHHHHHHGGMTVAYIAIGSNLASPLEQVNAALKALGDIPESHILTVSSFYRTPPLGPQDQPDYLNAAVALETSLAPEELLNHTQRIELQQGRVRKAERWGPRTLDLDIMLFGNEVINTERLTVPHYDMKNRGFMLWPLFEIAPELVFPDGEMLRQILHTRAFDKLNKW

(19) United States
(12) Patent Application Publication
(10) Pub. No.: US 2003/0088061 A1
(43) Pub. Date: May 8, 2003

(54) Materials and methods to modulate ligand binding/enzymatic activity of alpha/beta proteins containing an allosteric regulatory site

(76) Inventor: Donald E. Staunton, Kirkland, WA (US)

Correspondence Address: MARSHALL, GERSTEIN & BORUN, 6300 SEARS TOWER, 233 SOUTH WACKER, CHICAGO, IL, 60606-6357, US

(21) Appl. No.: 09/976,935

(22) Filed: Oct. 12, 2001

Related U.S. Application Data
(60) Provisional application No. 60/239,750, filed on Oct. 12, 2000.

(51) Int. Cl. A61K 31/165; C07K 14/435

(52) U.S. Cl. 530/350; 514/617

(57) ABSTRACT

Methods of modulating binding between an α/β protein and a binding partner are provided, along with methods of identifying modulators and their use.

### CROSS REFERENCE TO RELATED APPLICATION

This application claims the benefit of U.S. Provisional Application Serial No. 60/239,750, filed Oct. 12, 2000.

### FIELD OF THE INVENTION

The present invention provides materials and methods to regulate binding activity of alpha/beta (α/β) molecules comprising an allosteric regulatory site.

### BACKGROUND OF THE INVENTION

The alpha/beta (α/β) domain superfamily of proteins includes approximately ninety-seven families identified by specific fold structures. Proteins in the superfamily generally possess distinctive fold structures such as a TIM barrel, a horsehead fold or a beta-alpha-beta structure wherein a central beta sheet is surrounded by alpha helices, and is formed from multiple beta strand domains arranged in a parallel, anti-parallel or mixed orientation.

Many members of the superfamily, including proteins comprising an integrin I domain, von Willebrand factor comprising A domain structures, and various enzymes, have an open twisted beta sheet which gives rise to a fold in the protein's three dimensional structure. This fold is commonly referred to as a Rossmann fold, a Rossmann-like fold, or a dinucleotide binding fold. Many functionally diverse proteins contain Rossman folds, and these proteins can be identified using the SCOP, SMART, and CATH databases. A prototypic Rossmann fold is found at the site of NADP binding in glyceraldehyde-3-phosphate dehydrogenase.

Many Rossmann domains include a functional site on the “upper face” of the central beta sheet. This site in, for example, integrin I domains, Rho/Rac GTPases, and heterotrimeric GTPases, permits coordinated metal ion binding. In at least some integrin I domains, the bound metal ion forms a critical direct contact with a bound ligand and this site of metal ion binding has been designated the metal ion dependent adhesion site (MIDAS). Metal ion binding sites in other proteins are also proximal to ligand binding, including, for example, GTP/GDP binding to GTPases, and cofactor (i.e., NAD and FAD) binding to the bacterial protein ENR. Previous work has shown that for at least some proteins, including GTPases, LFA-1 [Huth, et al., Proc. Natl. Acad. Sci. (USA) 97:5231-5236 (2000)], Mac-1 [Oxvig, et al., Proc. Natl. Acad. Sci. (USA) 96:2215-20 (1999)] and Alpha2 [Emsley, et al., Cell 101:47-56 (2000)], ligand binding in the MIDAS region requires a conformation change between the active and inactive state of the protein.

The integrin I domain structure has been characterized in detail. Among the integrins in which I domain structures have been identified, primary amino acid sequence comparison indicates that overall homology can vary widely among different integrin family members. Despite this divergence in homology, some residues are highly conserved in many integrins. Further, it has remained unclear whether the observed divergence in amino acid sequence homology gives rise to substantial differences in tertiary structure of the I domain within the individual subunits or the quaternary structure in the heterodimers.

The I domains for α_{M} [Lee et al., Cell 80:631-638 (1995)], α_{L} [Qu et al., Structure 4:931-942 (1996)], α₁ [Rich, J. Biol. Chem., 274:24906-24913 (1999)], and α₂ [Emsley et al., J. Biol. Chem., 272:28512-28517 (1997)] have been crystallized, thereby permitting detailed analysis of previously speculated functional regions. The α_{M} crystalline structure clearly identified a Rossmann fold including a ligand-binding crevice formed along the top of the central, hydrophobic beta sheet, wherein the beta sheet is surrounded by multiple amphipathic α helices [Dickeson, et al., Cell. Mol. Life. Sci. 54:556-566 (1998)]. Consistent with previous observations, crystalline I domains for both α_{M} and α_{L} have also been shown to include a MIDAS region.

General structural observations from the crystalline α_{M} I domain appear to correlate to the crystalline structure of α_{L}. These observations clearly indicate that α_{L} undergoes a conversion from an inactive to an active state before ligand binding can occur. This observation has been confirmed in NMR studies wherein ICAM-1 binding to the α_{L} I domain was shown to require positional perturbations of amino acid residues in the α_{L} MIDAS region, as well as in a second region, still within the I domain but distal to the MIDAS region [Huth, et al., Proc. Natl. Acad. Sci. (USA) 97:5231-5236 (2000)].

Site directed mutagenesis in this second region has indicated that residues therein are not part of the ICAM-1 binding site, i.e., these residues do not interact directly with the ligand, but that these residues do, at least in part, play a role in regulating ICAM-1 binding. Amino acid residues that comprise this region have been designated the I domain allosteric site (IDAS) [Id.], and it is postulated that this region undergoes and/or induces a functionally relevant conformational shift that may be modulated by a small molecule. If the overall tertiary structure is conserved in the I or A domains of other proteins, such a site could provide an attractive target for modulating ligand binding for these proteins.

Furthermore, the crystal structure of the entire extracellular region of alphaVbeta3, an integrin, was recently reported [Cousin, Science, 293:1743-1746 (Sep. 7, 2001)]. The crystal structure confirms predictions that the beta subunit of all integrins contains an I domain. Because this I domain has been implicated in regulating integrin function, it is an additional potential site for modulating ligand binding for these proteins. Identification of such regulatory regions provides means by which modulators, i.e., agonists and antagonists, of ligand binding can be identified. Identification of such modulators provides candidate compounds that can provide protection against, and relief from, the myriad of pathological states associated with aberrant activity of α/β proteins.

Accordingly, there exists a need in the art to identify modes of modulating α/β proteins, which have a wide variety of functions and primary structures, in such a manner as to influence their biological activity.

### SUMMARY OF THE INVENTION

In one aspect, the present invention provides methods of modulating binding interaction between a first molecule which is not LFA-1 or an I domain-containing fragment thereof, and a binding partner molecule, said first molecule comprising an α/β domain structure, said α/β structure comprising an allosteric regulatory site, said method comprising the step of contacting said first molecule with an allosteric effector molecule that interacts with said allosteric regulatory site and promotes a conformation in a ligand binding domain of said α/β structure that modulates binding between said first molecule and said binding partner molecule. As used herein, the term “α/β structure” for a molecule refers to a general class of molecules that comprise a characteristic structure which is not necessarily indicative of, for example, molecules having multiple subunits which are designates as α and β subunits. This general class of molecules, however, can include molecules having multiple subunits which are designates as α and β subunits. The invention further provides methods of modulating binding interaction between a first molecule which is not LFA-1 or an I domain-containing fragment thereof, and a binding partner molecule, said first molecule comprising an α/β domain structure, said α/β structure comprising an allosteric regulatory site, said method comprising the step of contacting said first molecule with an allosteric effector molecule, said allosteric effector molecule comprising a diaryl compound, said diaryl compound interacting with said allosteric regulatory site and promoting a conformation in a ligand binding domain of said α/β structure that modulates binding between said first molecule and said binding partner molecule. In another aspect, the invention provides methods of modulating binding interaction between a first molecule which is not LFA-1 or an I domain-containing fragment thereof, and a binding partner molecule, said first molecule comprising an α/β domain structure, said α/β structure comprising an allosteric regulatory site, said method comprising the step of contacting said first molecule with an allosteric effector molecule, said allosteric effector molecule selected from the group consisting of diaryl sulfide compounds and diarylamide compounds, said allosteric effector molecule interacting with said allosteric regulatory site and promoting a conformation in a ligand binding domain of said α/β structure that modulates binding between said first molecule and said binding partner molecule.

In one embodiment, methods of the invention utilize a first molecule which comprises a Rossmann fold structure, said Rossmann fold structure comprising said allosteric regulatory site. As used herein, the term Rossmann fold structure encompasses Rossmann-like fold structures and dinucleotide fold structures, as is known in the art. In the methods the Rossmann fold structure in the first molecule comprises a β sheet having β sheet strands positioned in a 321456 or 231456 orientation. Alternatively, the Rossmann fold structure in the first molecule comprises a β sheet having β sheet strands positioned in a 3214567 orientation. In another aspect, the Rossmann fold structure in said first molecule comprises a β sheet having β sheet strands positioned in a 32145 orientation. As used herein, the term orientation refers to the positioning of the individual strands of a β sheet in a parallel, antiparallel or mixed configuration. Preferably, methods employ a first molecule which comprises an I domain structure or an A domain structure.

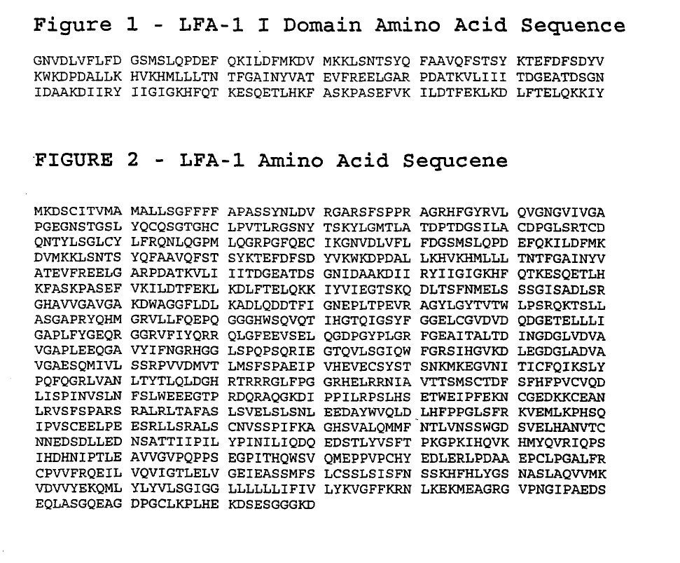

The invention further provides methods of modulating binding interaction between a first molecule and a binding partner molecule, said first molecule having an amino acid sequence which exhibits less than about 90% identity to the LFA-1 I domain amino acid sequence set out in FIG. 1, said first molecule comprising an α/β structure, said α/β domain structure comprising an allosteric regulatory site, said method comprising the step of contacting said first molecule with an allosteric effector molecule that interacts with said allosteric regulatory site and promotes a conformation in a ligand binding domain of said α/β structure that modulates binding between said first molecule and said binding partner molecule. The allosteric regulatory sites of the present invention include “I-like domains” or “IDAS-like domains,” as well as IDAS domains. As used herein, the terms I-like domains and IDAS-like domains refer to regulatory sites discrete (i.e., distinguishable) from the MIDAS region (in MIDAS-containing molecules), and discrete (i.e., distinguishable) from ligand, substrate or co-factor binding sites, that do not necessarily include a complete I domain per se, but do undergo and/or induce a functionally relevant conformational shift that may be modulated by a small molecule to increase or decrease binding between a first molecule and a binding partner molecule. In another aspect, the invention provides methods of modulating binding interaction between a first molecule and a binding partner molecule, said first molecule having an amino acid sequence which exhibits less than about 90% identity to the LFA-1 I domain amino acid sequence set out in FIG. 1, said first molecule comprising an α/β structure, said α/β domain structure comprising an allosteric regulatory site, said method comprising the step of contacting said first molecule with an allosteric effector molecule, said allosteric effector molecule comprising a diaryl compound, said diaryl compound interacting with said allosteric regulatory site and promoting a conformation in a ligand binding domain of said α/β structure that modulates binding between said first molecule and said binding partner molecule. In still another aspect, the invention provides methods of modulating binding interaction between a first molecule and a binding partner molecule, said first molecule having an amino acid sequence which exhibits less than about 90% identity to the LFA-1 I domain amino acid sequence set out in FIG. 1, said first molecule comprising an α/β domain structure, said α/β structure comprising an allosteric regulatory site, said method comprising the step of contacting said first molecule with an allosteric effector molecule, said allosteric effector molecule selected from the group consisting of diaryl sulfide compounds and diarylamide compounds, said allosteric effector molecule interacting with said allosteric regulatory site and promoting a conformation in a ligand binding domain of said α/β structure that modulates binding between said first molecule and said binding partner molecule. In a preferred embodiment, each of the methods the first molecule has an amino acid sequence that exhibits a percent identity with respect to the LFA-1 I domain amino acid sequence less than about 40%, about 45%, about 50%, about 55%, about 60%, about 65%, about 70%, about 75%, about 80%, about 85%, or about 90%. In another aspect, the first molecule comprises a Rossmann fold structure, said Rossmann fold structure comprising an allosteric regulatory site and the first molecule has an amino acid sequence that exhibits a percent identity with respect to the LFA-1 I domain amino acid sequence less than about 40%, about 45%, about 50%, about 55%, about 60%, about 65%, about 70%, about 75%, about 80%, about 85%, or about 90%. In another aspect, the methods of the invention utilize a first molecule wherein the Rossmann fold structure in said first molecule comprises a β sheet having β sheet strands positioned in a 321456 or 231456 orientation and the first molecule has an amino acid sequence that exhibits a percent identity with respect to the LFA-1 I domain amino acid sequence less than about 40%, about 45%, about 50%, about 55%, about 60%, about 65%, about 70%, about 75%, about 80%, about 85%, or about 90%. In another aspect, the methods use a protein wherein the Rossmann fold structure in said first molecule comprises a β sheet having β sheet strands positioned in a 3214567 orientation and the first molecule has an amino acid sequence that exhibits a percent identity with respect to the LFA-1 I domain amino acid sequence less than about 40%, about 45%, about 50%, about 55%, about 60%, about 65%, about 70%, about 75%, about 80%, about 85%, or about 90%. In another aspect, the method utilize a first molecule with a Rossmann fold structure comprising a β sheet having β sheets strands positioned in a 32145 orientation, and the first molecule has an amino acid sequence that exhibits a percent identity with respect to the LFA-1 I domain amino acid sequence less than about 40%, about 45%, about 50%, about 55%, about 60%, about 65%, about 70%, about 75%, about 80%, 85%, or about 90%. Preferably, the first molecule comprises an I domain structure and the first molecule has an amino acid sequence that exhibits a percent identity with respect to the LFA-1 I domain amino acid sequence less than about 40%, about 45%, about 50%, about 55%, about 60%, about 65%, about 70%, about 75%, about 80%, about 85%,or about 90%. In another preferred embodiment, the first molecule comprises an A domain structure and the first molecule has an amino acid sequence that exhibits a percent identity with respect to the LFA-1 I domain amino acid sequence less than about 40%, about 45%, about 50%, about 55%, about 60%, about 65%, about 70%, about 75%, about 80%, about 85%, or about 90%.

In methods of the invention, the modulator promotes a conformation in the ligand binding domain of said first molecule that increases binding between said first molecule and said binding partner molecule, and in one aspect, the increase in binding between the first molecule and the second molecule results in increased enzymatic activity of the first molecule. In another embodiment, the modulator promotes a conformation in the ligand binding domain of said first molecule that decreases binding between said first molecule and said binding partner molecule and the decrease in binding between the first molecule and the second molecule results in decreased enzymatic activity of the first molecule.

Methods include use of a first molecule selected from the group consisting of the proteins set forth in Table 1 as well as other proteins which comprise I or A domains, G proteins, heterotrimeric G proteins, and tubulin GTPase. Preferably, methods of the invention utilize a first molecule selected from the group consisting of the proteins set forth in Table 1. In one aspect, the first molecules is a eukaryotic molecule. Preferably, the first molecule is a human molecule. In another aspect, the first molecule is a prokaryotic molecule. In one embodiment, the first molecule is a bacterial molecule.

More preferably, the first molecule is selected from the group consisting of α_{M}β₂, complement protein C2, complement protein Factor B, α_{E}β₇, α₄β₇, α_{V}β₃, α₄β₁, α_{d}β₂, von Willebrand factor, Rac-1, HPPK, ftsZ, and ENR. In methods wherein the first molecule is α_{M}β₂ and the binding partner protein is fibrinogen; the first molecule is α_{M}β₂ and the binding partner protein is iC3b; the first molecule is α_{E}β₇ and the binding partner protein is E-cadherin; the first molecule is α₄β₇ and the binding partner protein is MadCAM-1; the first molecule is α_{V}β₃ and the binding partner protein is vitronectin; the first molecule is α₄β₁ and the binding partner protein is VCAM; the first molecule is α_{d}β₂ and the binding partner protein is VCAM; the first molecule is von Willebrand factor and the binding partner protein is gpIb; the first molecule is complement protein C2 and the binding partner protein is complement protein C4b; the first molecule is complement protein Factor B and the binding partner protein is complement protein C3b; the first molecule is Rac-1 and the binding partner is GTP; the first molecule is HPPK and the binding partner is ATP or HMDP; the first molecule is ftsZ and the binding partner is GTP; and the first molecule is ENR and the binding partner is NADH.

### DETAILED DESCRIPTION OF THE INVENTION

In one aspect, the present invention provides methods of modulating binding interaction between a first molecule which is not LFA-1 or an I domain-containing fragment or mimetics thereof, and a binding partner molecule, said first molecule comprising an α/β structure, said α/β structure comprising an allosteric regulatory site, said method comprising the step of contacting said first molecule with an allosteric effector molecule that interacts with said allosteric regulatory site and promotes a conformation in a ligand binding domain of said α/β structure that modulates binding between said first molecule and said binding partner molecule. As used herein, “binding partner molecules” includes ligands, substrates and cofactor, the binding of which is required to effect one or more biological activity of the first molecule. An I domain fragment of LFA-1 is a polypeptide portion or fragment (i.e., a polypeptide that is less than full length LFA-1 as set out in FIG. 2) of LFA-1 that comprises (i) the I domain of LFA-1, or (ii) a portion of the LFA-1 I domain that maintains biologically active features of the LFA-1 I domain. Synthetic mimetics of the LFA-1 I domain, including peptidomimetics which replicate or affect one or more biological activities of the LFA-1 I domain, are also included in this definition. The α/β superfamily of proteins includes those proteins having an beta-alpha-beta structure wherein a central beta sheet domain is flanked on both sides of the sheet by one or more alpha helix domains.

In another aspect, the present invention provides methods of modulating binding interaction between a first molecule which is not LFA-1 or an I domain-containing fragment or mimetics thereof, and a binding partner molecule, said first molecule comprising a Rossmann fold structure, said Rossmann fold structure comprising an allosteric regulatory site, said method comprising the step of contacting said first molecule with an allosteric effector molecule that interacts with said allosteric regulatory site and promotes a conformation in a ligand binding domain of said Rossmann fold structure that modulates binding between said first molecule and said binding partner molecule. A Rossmann fold structure in a protein comprises a beta sheet structure wherein individual beta sheet domains of the protein are positioned in either parallel, antiparallel, or mixed orientations. In preferred aspects of the present invention, the beta sheet of the first molecule is comprised of individual beta sheet strands. Numerical designations for the individual beta sheet strands are assigned according to their position in the primary amino acid sequence of the first protein, with the first beta sheet strand being that one closest to the amino terminus of the protein sequence. Rossmann fold structures are further characterized by the presence of a ligand binding fold, pocket, or site in the three dimensional structure of the beta sheet that is generally positioned at the “top” of the beta sheet structure.

In another aspect, the present invention provides methods of modulating binding interaction between a first molecule which is not LFA-1 or an I domain-containing fragment or mimetic thereof, and a binding partner molecule, said first molecule comprising a Rossmann fold structure, said Rossmann fold structure comprising a β sheet having β strands positioned in a 321456 or 231456 orientation and an allosteric regulatory site, said method comprising the step of contacting said first molecule with an allosteric effector molecule that interacts with said allosteric regulatory site and promotes a conformation in a ligand binding domain of said Rossmann fold structure that modulates binding between said first molecule and said binding partner molecule. In another aspect, the present invention provides methods of modulating binding interaction between a first molecule which is not LFA-1 or an I domain-containing fragment or mimetic thereof, and a binding partner molecule, said first molecule comprising a Rossmann fold structure, said Rossmann fold structure comprising a β sheet having β strands positioned in a 3214567 orientation and an allosteric regulatory site, said method comprising the step of contacting said first molecule with an allosteric effector molecule that interacts with said allosteric regulatory site and promotes a conformation in a ligand binding domain of said Rossmann fold structure that modulates binding between said first molecule and said binding partner molecule. The present invention also provides methods of modulating binding interaction between a first molecule which is not LFA-1 or an I domain-containing fragment or mimetic thereof, and a binding partner molecule, said first molecule comprising a Rossmann fold structure, said Rossmann fold structure comprising a β sheet having β strands positioned in a 32145 orientation and an allosteric regulatory site, said method comprising the step of contacting said first molecule with an allosteric effector molecule that interacts with said allosteric regulatory site and promotes a conformation in a ligand binding domain of said Rossmann fold structure that modulates binding between said first molecule and said binding partner molecule. Numerical designations for individual beta sheets in the first molecule are as described above.

In another aspect, the present invention provides methods of modulating binding interaction between a first molecule which is not LFA-1 or an I domain-containing fragment or mimetic thereof, and a binding partner molecule, said first molecule comprising an I domain structure, said I domain structure comprising an allosteric regulatory site, said method comprising the step of contacting said first molecule with an allosteric effector molecule that interacts with said allosteric regulatory site and promotes a conformation in a ligand binding domain of said I domain structure that modulates binding between said first molecule and said binding partner molecule. I domain structures are known in the art to comprise approximately 200 amino acids as exemplified by the domains identified in a number of integrins [See Dickeson, et al., Cell. Mol. Life Sci. 54:556-566 (1998)].

The present invention also provides methods of modulating binding interaction between a first molecule which is not LFA-1 or an I domain-containing fragment thereof, and a binding partner molecule, said first molecule comprising an A domain structure, said A domain structure comprising an allosteric regulatory site, said method comprising the step of contacting said first molecule with an allosteric effector molecule that interacts with said allosteric regulatory site and promotes a conformation in a ligand binding domain of said A domain structure that modulates binding between said first molecule and said binding partner molecule. A domain motifs are known in the art to share homology with I domains and are exemplified by the domains found in von Willebrand factor.

The present invention also provides methods of modulating binding interaction between a first molecule and a binding partner molecule, said first molecule having an amino acid sequence which exhibits less than about 90% identity to the LFA-1 I domain amino acid sequence [set out in FIG. 1], said first molecule comprising an α/β structure, said α/β structure comprising an allosteric regulatory site, said method comprising the step of contacting said first molecule with an allosteric effector molecule that interacts with said allosteric regulatory site and promotes a conformation in a ligand binding domain of said α/β structure that modulates binding between said first molecule and said binding partner molecule. Identity as used herein can be calculated using basic BLAST analysis using default parameters. Values for percent identity reflect one-to-one correspondence between amino acid residues across the entire LFA-1 sequence I domain as set out in FIG. 1 and a region of amino acid residues of the same or similar length in the first molecule. In another embodiment of the method, the first molecule has an amino acid sequence that exhibits a percent identity with respect to the LFA-1 I domain amino acid sequence of less than about 40%, about 45%, about 50%, about 55%, about 60%, about 65%, about 70%, about 75%, about 80%, about 85%, or about 90%.

In still another aspect, the present invention provides methods of modulating binding interaction between a first molecule and a binding partner molecule, said first molecule having an amino acid sequence which exhibits less than about 90% identity to the LFA-1 I domain amino acid sequence [set out in FIG. 1], said first molecule comprising a Rossmann fold structure, said Rossmann fold structure comprising an allosteric regulatory site, said method comprising the step of contacting said first molecule with an allosteric effector molecule that interacts with said allosteric regulatory site and promotes a conformation in a ligand binding domain of said Rossmann fold structure that modulates binding between said first molecule and said binding partner molecule. In alternative embodiments of the method, the first molecule has an amino acid sequence that exhibits a percent identity with respect to the LFA-1 I domain amino acid sequence of less than about 40%, about 45%, about 50%, about 55%, about 60%, about 65%, about 70%, about 75%, about 80%, about 85%, or about 90%.

In another aspect, the present invention provides methods of modulating binding interaction between a first molecule and a binding partner molecule, said first molecule having an amino acid sequence which exhibits less than about 90% identity to the LEA-1 I domain amino acid sequence [set out in FIG. 1], said first molecule comprising a Rossmann fold structure with β sheets strands positioned in a 321456 or 231456 orientation and an allosteric regulatory site, said method comprising the step of contacting said first molecule with an allosteric effector molecule that interacts with said allosteric regulatory site and promotes a conformation in a ligand binding domain of said Rossmann fold structure that modulates binding between said first molecule and said binding partner molecule. In alternative embodiments of the method, the first molecule has an amino acid sequence that exhibits a percent identity with respect to the LFA-1 I domain amino acid sequence of less than about 40%, about 45%, about 50%, about 55%, about 60%, about 65%, about 70%, about 75%, about 80%, about 85%, or about 90%.

In still another aspect, the present invention provides methods of modulating binding interaction between a first molecule and a binding partner molecule, said first molecule having an amino acid sequence which exhibits less than about 90% identity to the LFA-1 I domain amino acid sequence [set out in FIG. 1], said first molecule comprising a Rossmann fold structure, said Rossmann fold structure with β sheet strands positioned in a 3214567 orientation and an allosteric regulatory site, said method comprising the step of contacting said first molecule with an allosteric effector molecule that interacts with said allosteric regulatory site and promotes a conformation in a ligand binding domain of said Rossmann fold structure that modulates binding between said first molecule and said binding partner molecule. In alternative embodiments of the method, the first molecule has an amino acid sequence that exhibits a percent identity with respect to the LFA-1 I domain amino acid sequence of less than about 40%, about 45%, about 50%, about 55%, about 60%, about 65%, about 70%, about 75%, about 80%, about 85%, or about 90%.

The present invention also provides methods of modulating binding interaction between a first molecule and a binding partner molecule, said first molecule having an amino acid sequence which exhibits less than about 90% identity to the LFA-1 I domain amino acid sequence [set out in FIG. 1], said first molecule comprising a Rossmann fold structure β sheet strands positioned in a 32145 orientation and an allosteric regulatory site, said method comprising the step of contacting said first molecule with an allosteric effector molecule that interacts with said allosteric regulatory site and promotes a conformation in a ligand binding domain of said Rossmann fold structure that modulates binding between said first molecule and said binding partner molecule. In alternative embodiments of the method, the first molecule has an amino acid sequence that exhibits a percent identity with respect to the LFA-1 I domain amino acid sequence of less than about 40%, about 45%, about 50%, about 55%, about 60%, about 65%, about 70%, about 75%, about 80%, about 85%, or about 90%.

The present invention further provides methods of modulating binding interaction between a first molecule and a binding partner molecule, said first molecule having an amino acid sequence which exhibits less than about 90% identity to the LFA-1 I domain amino acid sequence [set out in FIG. 1], said first molecule comprising an I domain structure, said I domain structure comprising an allosteric regulatory site, said method comprising the step of contacting said first molecule with an allosteric effector molecule that interacts with said allosteric regulatory site and promotes a conformation in a ligand binding domain of said I domain structure that modulates binding between said first molecule and said binding partner molecule. In alternative embodiments of the method, the first molecule has an amino acid sequence that exhibits a percent identity with respect to the LFA-1 I domain amino acid sequence of less than about 40%, about 45%, about 50%, about 55%, about 60%, about 65%, about 70%, about 75%, about 80%, about 85%, or about 90%.

In another aspect, the present invention provides methods of modulating binding interaction between a first molecule and a binding partner molecule, said first molecule having an amino acid sequence which exhibits less than about 90% identity to the LFA-1 I domain amino acid sequence [set out in FIG. 1], said first molecule comprising an A domain structure, said A domain structure comprising an allosteric regulatory site, said method comprising the step of contacting said first molecule with an allosteric effector molecule that interacts with said allosteric regulatory site and promotes a conformation in a ligand binding domain of said A domain structure that modulates binding between said first molecule and said binding partner molecule. In alternative embodiments of the method, the first molecule has an amino acid sequence that exhibits a percent identity with respect to the LFA-1 I domain amino acid sequence of less than about 40%, about 45%, about 50%, about 55%, about 60%, about 65%, about 70%, about 75%, about 80%, about 85%, or about 90%.

In each of the methods of the present invention, the modulator promotes a conformation in the ligand binding domain of said first molecule that increases binding between said first molecule and said binding partner molecule. Alternatively, the modulator promotes a conformation in the ligand binding domain of said first molecule that decreases binding between said first molecule and said binding partner molecule. Preferably, the methods include a first molecule selected from the group consisting of the molecules set out in Table 1 or otherwise described herein. Most preferably, methods utilize a first molecule selected from the group consisting of α_{M}β₂, complement protein C2, complement protein Factor B, α_{E}β₇, α₄β₇, α_{V}β₃, α₄β₁,α_{d}β₂ von Willebrand factor, Rac-1, HPPK, ftsZ, and ENR. Furthermore, preferably, the methods and compositions of the present invention use a modulator that is a diaryl compound. More preferably, the methods and compositions of the present invention use a modulator that is selected from diaryl sulfide compounds and diarylamide compounds. Most preferably, the methods and compositions of the present invention use a modulator that is a diaryl sulfide compound.

In methods wherein the first molecule is α_{M}β₂, the preferred binding partner protein is fibrinogen, and a preferred modulator is selected from the group consisting of Cmpd S, Cmpd R, Cmpd N, Cmpd O, Cmpd P, Cmpd Q, Cmpd L, Cmpd V, Cmpd F, Cmpd AA, and Cmpd AC as set out in Table 2. In methods wherein the first molecule is α_{M}β₂, an alternative preferred binding partner protein is iC3b and a preferred modulator is selected from the group consisting of Cmpd H, Cmpd I and Cmpd C. In methods wherein the first molecule is α_{E}β₇, the preferred binding partner protein is E-cadherin and a preferred modulator is selected from the compounds set out in Table 2 herein. In methods wherein the first molecule is α₄β₇, the preferred binding partner protein is MAdCAM-1. In methods wherein the first molecule is α_{V}β₃, the preferred binding partner protein is vitronectin. In methods wherein the first molecule is α₄β₁, the preferred binding partner protein is VCAM. In methods wherein the first molecule is α_{d}β₂, the preferred binding partner protein is VCAM. In methods wherein the first molecule is von Willebrand factor, the preferred binding partner protein is gpIb. In methods wherein the first molecule is complement protein C2, the preferred binding partner protein is complement protein C4b. In methods wherein the first molecule is complement protein Factor B, the preferred binding partner protein is complement protein C3b. In methods wherein the first molecule is either α₁β₁, α₂β₁, α₁₁β₁ the preferred binding partner is collagen. In methods wherein the first molecule is α₂β₁, the preferred binding partner is collagen and a preferred modulator is selected from the group of compounds set out in Table 2 herein. In methods wherein the first molecule is Rac-1, the preferred binding partner is GDP/GTP and a preferred modulator GTP. In methods wherein the first molecule is HPPK, the preferred binding partner is ATP or HMDP. In methods wherein the first molecule is ftsZ, the preferred binding partner is GTP. In methods wherein the first molecule is ENR, the preferred binding partner is NADH.

Methods of the present invention include those wherein the first molecule, the binding partner molecule or both are isolated proteins, or binding fragments thereof, obtained from natural sources or from cells modified to express the molecules as heterologous proteins. The methods also embrace use of the first molecule, or a binding fragment thereof, the binding partner molecule, or a binding fragment thereof, both which are expressed on the surface of cells which express the molecules as homologous proteins or on the surface of cells which have been modified to express heterologous proteins. In vivo and in vitro methods are contemplated.

In vivo methods are expected to alleviate and/or prevent pathological states which arise from aberrant binding activity between the first molecule and the binding partner molecule. For example, indications associated with inappropriate complement activation for which methods of the present invention axe expected to alleviate or prevent include: (i) diseases involving antibody/complement deposition which includes systemic lupus erythematosus (SLE), Goodpasture's disease, rheumatoid arthritis, myasthenia gravis, autoimmune hemolytic anemia, autoimmune thrombocytopenic purpura, and Rasmussen's encephalitis; (ii) diseases involving ischemia-reperfusion injury, including stroke, myocardial infarction, cardiac pulmonary bypass, acute hypovolemic disease, renal failure, and allotransplantation; (iii) central nervous system pathologies such as Alzheimer's disease and multiple sclerosis; and (iv) miscellaneous indications such as trauma, chemical or thermal injury, and xenotransplantation.

Likewise, inhibitors of alpha 1, alpha 2, and alpha 11 are also expected to be useful for treating cancer. During metastasis, tumor cells must pass through the extracellular matrix prior to intravasation and following extravasation. Migration through these regions is dependent on integrin activity. In addition, it has been shown that blocking of α₁ or α₂ activity with monoclonal antibodies [Locher et al., Mol. Biol. Cell. 10:271-282 (1999)] or removal of α₁ activity in a knockout mouse [Pozzi, et al., Proc. Natl. Acad. Sci. (USA) 97:2202-2207 (2000)] results in changes in matrix metalloproteinase (MMP) levels. MMPs are extracellular matrix-degrading enzymes which have been proposed to play a role in a variety of types of cancer. [For a review, see Nelson, et. al., J. Clin. Oncol. 18:1135-1149 (2000)]. Inhibitors of MMPs are currently being tested for clinical utility in treating many types of cancer. To date, MMP inhibitors have not been as effective in human trials as in animal models. Modulating MMP expression by inhibiting integrin activity can prove to be more effective by differentially modulating different MMP levels and by specifically targeting this MMP modulation to α₁, α₂, or α₁₁ expressing cells.

More particularly, it has been demonstrated that alpha 11 is expressed on foamy macrophages in atherosclerotic plaques as well as in a subset of macrophages in synovium from a patient with rheumatoid arthritis. No expression has been seen in non-activated monocyte derived macrophages. Inhibitors of alpha 11/ligand binding interactions could therefore be useful for reducing migration and/or signaling events of macrophages that are associated with different inflammatory processes. Accordingly, alpha 11 inhibitors could represent useful therapeutics for treating inflammatory diseases, including atherosclerosis and rheumatoid arthritis.

Similarly, alpha 1 and alpha 2 integrins have been shown to be upregulated on certain cells (including T cells and monocytes) following stimulation. It has also been demonstrated that blocking interactions between alpha 1 or alpha 2 and their ligands using monoclonal antibodies inhibited inflammatory responses in mouse models of delayed-type hypersensitivity, contact hypersensitivity and arthritis [deFougerolles et. al. J. Clin. Invest. 105:721-729(2000)]. Antagonists of alpha1 and alpha 2 may inhibit inflammation through a variety of mechanisms including inhibiting cell migration, cell proliferation and the production of inflammatory mediators such as matrix metalloproteinase 3, tumor necrosis factor alpha and interleukin-1. Accordingly, small molecule inhibitors or antagonists of alpha1 and alpha2 associations (ligand binding), i.e., allosteric effector molecules, could be useful for the treatment of inflammatory diseases such as arthritis, fibrotic diseases and cancer.

Fibrotic disease states are characterized by the excessive production of fibrous extracellular matrix by certain cell types that are inappropriately activated. It is believed that the mechanism of fibrous extracellular matrix formation involves, at least in part, α/β protein activity. Accordingly, by inhibiting α/β proteins, the present invention provides methods and compositions for the treatment and prevention of various fibrotic disease states, including scleroderma (morphea, generalized morphea, linear scleroderma), keloids, hypertrophic scar, nodular fascuitis, eosinophilic fasciitis, Dupuytren's contracture, kidney fibrosis, pulmonary fibrosis, chemotherapy/radiation induced lung fibrosis, atherosclerotic plaques, inflammatory bowel disease, Crohn's disease, arthritic joints, invasive breast carcinoma desmosplasis, dermatofibromas, endothelial cell expression, angiolipoma, angioleiomyoma, sarcoidosis, cirrhosis, idiopathic interstitial lung disease, idiopathic pulmonary fibrosis (4 pathologic types), collagen vascular disease associated lung syndromes, cryptogenic organizing pneumonia, Goodpasture's syndrome, Wegener's granulomatosis, eosinophilic granuloma, iatrogenic lung disease, pneumoconioses (asbestosis, silicosis), hypersensitivity pneumonitides (farmer's lung, bird fancier's lung, etc.), interstitial pulmonary fibrosis, chemical pneumonitis, hypersensitivity pneumonitis and the like.

With respect to bacterial proteins, ENR is already a target for anti-tuberculosis drugs and a target of the broad spectrum biocide triclosan. Small molecules would therefore be useful in drug resistant tuberculosis. Moreover, the activity spectrum of ENR and DapB inhibitors would be useful as Gram negative inhibitors. Furthermore, because ERA-GTPase is highly conserved among bacteria, inhibitors would be useful against a broad spectrum of bacteria, depending on permeability. In addition, inhibitors of the various bacterial proteins would be useful for treating bacterial diseases involving Gram negative bacteria and infections with undefined bacterial pathogens.

Other chemotherapeutics, such as sulfonamides, inhibit bacterial growth by antagonizing the de novo folate biosynthetic pathway [Mandell and Petri, Sulfonamides, Trimethoprim-sulfamethoxazole, Quinolones, and Agents for Urinary Tract Infections, in The Pharmacological Basis of Therapeutics (Goodman and Gilman eds., 1996)]. The primary goal of anti-folate therapy is to deplete the intracellular pools of reduced folate, resulting in the inhibition of DNA replication due to insufficient levels of thymidine [Hitchings and Baccanari, Design and Synthesis of Folate Antagonists as Antimicrobial Agents, in Folate Antagonists as Therapeutic Agents (1984)].

The enzyme 6-hydroxymethyl-7,8-dihydropterin pyrophosphokinase (HPPK) catalyzes the transfer of pyrophosphate from ATP to 6-hydroxy-7,8-dihydropterin (HMDP) in the de novo folate biosynthetic pathway [Richey and Brown, J. Biol. Chem., 244:1582-1592 (1969)]. HPPK is expressed in both Gram positive and Gram negative bacteria, fungi, and protozoa, but not in higher eukaryotes, and represents an important target for the development of antibiotics with anti-folate activity. By inhibiting HPPK, the present invention can provide methods and compositions for the treatment and prevention of various bacterial and fungal infections.

FtsZ is the product of an essential bacterial gene that is involved in cell division. FtsZ binds and hydrolyzes GTP, and when bound to GTP it forms long, linear polymers. The GTP-dependent polymerization of ftsZ is related to its function in bacterial cell division. During septation, ftsZ forms a ring to define the plane of cell division. Cells lacking ftsZ can not undergo septation, do not divide and die. FtsZ is highly conserved (approximately 60%) throughout the bacterial kingdom. Accordingly, by inhibiting ftsZ, the compositions and methods of the present invention provide broad-spectrum antibiotics. The atomic structure of ftsZ shows that it is an alpha/beta protein [Nogales et al., (1998) Nature Structural Biology 5:451-458].

Modulators of vWF binding are useful in treatment of thrombotic vascular diseases, such as myocardial infarction (MI) and thrombotic stroke. Acute administration of a vWF A1-domain binding antagonist can reduce the risk of coronary vascular occlusion in high risk patients such as those with unstable angina, or following PTCA or stent placement. Several gpIIb/IIIa antagonists have recently been approved for clinical use in these settings (ReoPro®, Itrafiban, sibrafiban). While these agents are effective, their use is accompanied by bleeding, thus limiting their effective dose. If the bleeding side effects of an A1-domain inhibitor are limited, it can be used chronically in individuals at risk for vascular occlusion. These individuals include patients with angina, claudication, and those with a history of MI or stroke. Abnormalities of vWF metabolism are the cause of the occlusive thrombus in thrombotic thrombocytopenic purpura, suggesting A1 domain inhibitors may also be useful in this setting.

Rac1, Rac2 and Rac3 are members of the Ras superfamily of small molecular weight (approximately 22-25 kDa) GTPases, many of which are α/β proteins [Edwards and Perkins, FEBS Lett 358:283 (1995); De Vos et al., Science 239:888 (1988); Worthylake et al., Nature 408:682 (2000)]. Primary amino acid sequence comparison indicates that the overall homology of the Rac proteins is about 88 to about 92 percent identical. It is known that Rac1 and Rac2 proteins play a crucial role in cell survival, proliferation, metastasis and reactive oxygen species (ROS) production [Symons, Curr. Opin. in Biotech., 6:668 (1995); and, Scita, EMBO J., 19(11):2393 (2000)]. Due to the importance of Rac proteins in the control of cell proliferation, antagonists of the Rac guanine nucleotide exchange reaction and, in particular, small molecules that interfere with the exchange of GDP for GTP of Rac1 in the presence of Tiam1, are of considerable interest for the methods and compositions of the present invention.

In view of the indications described above, the present invention further provides methods for alleviating or preventing a condition arising from aberrant binding between a first molecule that is not LFA-1 I or an I domain fragment thereof and a binding partner molecule, wherein said first molecule is an α/β protein selected from the group of proteins set forth in Table 1, said method comprising the steps of administering to an individual in need thereof an effective amount of a modulator of binding between said first molecule and said binding partner molecule. As used herein, the term effective amount refers to the administration of an amount of a modulator sufficient to achieve its intended purpose. More specifically, a “therapeutically effective amount” refers to an amount effective to treat or to prevent development of, or to alleviate the existing symptoms of, the subject being treated. Determination of the effective amounts is well within the capability of those skilled in the art, especially in light of the detailed disclosure provided herein.

In one aspect, the present invention provides methods of treatment wherein the α/β protein comprises a Rossmann fold. In another aspect, methods of treatment are provided wherein the Rossmann fold in the targeted protein includes five, six or seven β strands which makeup the central β sheet structure. When the Rossmann fold comprises five β strands, it is preferred that the positioning of the individual strands is 32145 as defined above. When the Rossmann fold comprises six β strands, it is preferred that the positioning of the individual strands is 321456 or 231456 as defined above. When the Rossmann fold comprises seven β strands, it is preferred that the positioning of the individual strands is 3214567 as defined above. Methods of treatment the present invention include those wherein the first molecule exhibits less than about 90% amino acid sequence identity with the I domain amino acid sequence of LFA-1 as set out in FIG. 1. Preferably, the first molecule will have a percent amino acid sequence identity with the I domain of LFA-1 less than about 40%, about 45%, about 50%, about 55%, about 60%, about 65%, about 70%, about 75%, about 80%, about 85%, or about 90%. Sequence identity for purposes of this aspect of the present invention is calculated using, for example, basic BLAST search analysis with default parameters.

The present invention also provides methods for identifying a modulator of binding between a first molecule that is not LFA-1 or an I domain fragment thereof and a binding partner molecule, wherein said first molecule is an α/β protein selected from the group of proteins set forth in Table 1, said method comprising the steps of measuring binding between the first molecule and the binding partner molecule in the presence and absence of a test compound, and identifying the test compound as a modulator of binding when a change in binding between the first molecule and the binding partner molecule is detected in the presence of the test compound as compared to binding in the absence of the test compound. In one aspect, the present invention provides methods wherein the α/β protein comprises a Rossmann fold. In another aspect, methods are provided wherein the Rossmann fold in the targeted protein includes five, six or seven β strands which makeup the central β sheet structure. When the Rossmann fold comprises five β strands, it is preferred that the positioning of the individual strands is 32145 as defined above. When the Rossmann fold comprises six β strands, it is preferred that the positioning of the individual strands is 321456 231456 as defined above. When the Rossmann fold comprises seven β strands, it is preferred that the positioning of the individual strands is 3214567 as defined above. Methods of the present invention include those wherein the first molecule exhibits less than about 90% amino acid sequence identity with the I domain amino acid sequence of LFA-1 as set out in FIG. 1. Preferably, the first molecule will have a percent amino acid sequence identity with the I domain of LFA-1 less than about 40%, about 45%, about 50%, about 55%, about 60%, about 65%, about 70%, about 75%, about 80%, about 85%, or about 90%. Sequence identity for purposes of this aspect of the present invention is calculated using, for example, basic BLAST search analysis with default parameters.

The present invention also provides modulators of binding between a first molecule that is not LFA-1 or an I domain fragment thereof and a binding partner molecule, wherein said first molecule is an α/β protein selected from the group of proteins set forth in Table 1. In one aspect, the modulators are those that affect binding of an α/β protein which comprises a Rossmann fold. In another aspect, modulators are provided which affect binding when the Rossmann fold in the targeted protein includes five, six or seven β strands which makeup the central β sheet structure. When the Rossmann fold comprises five β strands, it is preferred that the positioning of the individual strands is 32145 as defined above. When the Rossmann fold comprises six β strands, it is preferred that the positioning of the individual strands is 321456 or 231456 as defined above. When the Rossmann fold comprises seven β strands, it is preferred that the positioning of the individual strands is 3214567 as defined above. Modulators are also provided for a first molecule which exhibits less than about 90% amino acid sequence identity with the I domain amino acid sequence of LFA-1 as set out in FIG. 1. Preferably, the first molecule will have a percent amino acid sequence identity with the I domain of LFA-1 less than about 40%, about 45%, about 50%, about 55%, about 60%, about 65%, about 70%, about 75%, about 80%, about 85%, or about 90%. Sequence identity for purposes of this aspect of the present invention is calculated using, for example, basic BLAST search analysis with default parameters.

The present invention also provides compositions comprising a modulator. Preferred compositions are pharmaceutical compositions. The pharmaceutical compositions of the present invention comprise one or more modulators of the present invention, preferably further comprising a pharmaceutically acceptable carrier or diluent. The term “pharmaceutically acceptable carrier” as used herein refers to compounds suitable for use in contact with recipient animals, preferably mammals, and more preferably humans, and having a toxicity, irritation, or allergic response commensurate with a reasonable benefit/risk ratio, and effective for their intended use.

The present invention also provides modulators which exist in a prodrug form. The term “prodrug” as used herein refers to compounds which are rapidly transformed in vivo to the parent, or active modulator, compound, for example, by hydrolysis. A thorough discussion is provided in Higuchi, et al., Prodrugs as Novel Delivery Systems, vol. 14 of the A.C.S.D. Symposium Series, and in Roche (ed), Bioreversible Carriers in Drug Design, American Pharmaceutical Association and Pergamon Press, 1987, both of which are incorporated herein by reference. Prodrug design is discussed generally in Hardma, et al., (Eds), Goodman & Gilman's The Pharmacological Basis of Therapeutics, Ninth Edition, New York, N.Y. (1996), pp. 11-16. Briefly, administration of a drug is followed by elimination from the body or some biotransformation whereby biological activity of the drug is reduced or eliminated. Alternatively, a biotransformation process may lead to a metabolic by-product which is itself more active or equally active as compared to the drug initially administered. Increased understanding of these biotransformation processes permits the design of so-called “prodrugs” which, following a biotransformation, become more physiologically active in an altered state. Prodrugs are therefore pharmacologically inactive compounds which are converted to biologically active metabolites. In some forms, prodrugs are rendered pharmacologically active through hydrolysis of, for example, an ester or amide linkage, often times introducing or exposing a functional group on the prodrug. The thus modified drug may also react with an endogenous compound to form a water soluble conjugate which further increases pharmacological properties of the compound, for example, as a result of increased circulatory half-life.

As another alternative, prodrugs can be designed to undergo covalent modification on a functional group with, for example, glucuronic acid sulfate, glutathione, amino acids, or acetate. The resulting conjugate may be inactivated and excreted in the urine, or rendered more potent than the parent compound. High molecular weight conjugates may also be excreted into the bile, subjected to enzymatic cleavage, and released back into circulation, thereby effectively increasing the biological half-life of the originally administered compound.

Compounds of the present invention may exist as stereoisomers where asymmetric or chiral centers are present. Stereoisomers are designated by either “S” or “R” depending on the arrangement of substituents around a chiral carbon atom. Mixtures of stereoisomers are contemplated by the present invention. Stereoisomers include enantiomers, diastereomers, and mixtures thereof. Individual stereoisomers of compounds of the present invention can be prepared synthetically from commercially available starting materials which contain asymmetric or chiral centers or by preparation of racemic mixtures followed by separation or resolution techniques well known in the art. Methods of resolution include (1) attachment of a mixture of enantiomers to a chiral auxiliary, separation of the resulting mixture by recrystallization or chromatography, and liberation of the optically pure product from the auxiliary; (2) salt formation employing an optically active resolving agent, and (3) direct separation of the mixture of optical enantiomers on chiral chromatographic columns.

The pharmaceutical compositions of the present invention can be administered to humans and other animals by any suitable route. For example, the compositions can be administered orally, rectally, parenterally, intracisternally, intravaginally, intraperitoneally, topically (as by powders, ointments, or drops), bucally, or nasally. The term “parenteral” administration as used herein refers to modes of administration which include intravenous, intraarterial, intramuscular, intraperitoneal, intrasternal, intrathecal, subcutaneous and intraarticular injection and infusion.

Pharmaceutical compositions of this present invention for parenteral injection comprise pharmaceutically-acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as olive oils), and injectable organic esters such as ethyl oleate. Proper fluidity can be maintained, for example, by the use of coating materials such as lecithin, by the maintenance of the required particle size, in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

In some cases, in order to prolong the effect of the drug, it is desirable to slow the absorption of the drug from subcutaneous or intramuscular injection. This result may be accomplished by the use of a liquid suspension of crystalline or amorphous materials with poor water solubility. The rate of absorption of the drug then depends upon its rate of dissolution, which in turn may depend upon crystal size and crystalline form. Alternatively, delayed absorption of a parenterally administered drug from is accomplished by dissolving or suspending the drug in an oil vehicle.

Injectable depot forms are made by forming microencapsule matrices of the drug in biodegradable polymers such a polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The injectable formulations can be sterilized, for example, by filtration through a bacterial- or viral-retaining filter, or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In such solid dosage forms, the active compound is mixed with a least one inert, pharmaceutically-acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, gums (e.g. alginates, acacia) gelatin, polyvinylpyrrolidone, and sucrose, (c) humectants such as glycerol, (d) disintegrating agents such as agar-agar, calcium carbonate, potato or tapioca starch, alginic acid, certain silicates, and sodium carbonate, (e) solution retarding agents such a paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a part of the intestinal tract, optionally, in a delayed manner. Exemplary materials include polymers having pH sensitive solubility, including commercially available materials such as Eudragit®. Examples of embedding compositions which can be used include polymeric substances and waxes.

The active compounds can also be in micro-encapsulated form if appropriate, with one or more of the above-mentioned excipients.

Liquid dosage forms for oral administration include pharmaceutically-acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, may contain suspending agents such as, for example, ethoxylated isostearyl alcohols, polyoxyethylene sorbitol and sorbitani esters, microcrystalline cellulose, aluminum metahydroxide, bentonite, agar-agar, and tragacanth, and mixtures thereof.

Compositions for rectal or vaginal administration are preferably suppositories which can be prepared by mixing the compounds of the present invention with suitable non-irritating excipients or carriers such as cocoa butter, polyethylene glycol or suppository wax, which are solid at room temperature but liquid at body temperature. Accordingly, such carriers melt in the rectum or vaginal cavity, releasing the active compound.

Compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically-acceptable and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p. 33 et seq.

The compounds of the present invention may be used in the form of pharmaceutically-acceptable salts derived from inorganic or organic acids. “Pharmaceutically-acceptable salts” include those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically-acceptable salts are well known in the art. For example, S. M. Berge, et al., describe pharmaceutically-acceptable salts in detail in J. Pharmaceutical Sciences, 66:1 (1977), incorporated herein by reference in its entirety. The salts may be prepared in situ during the final isolation and purification of the compounds of the present invention or separately by reacting a free base function with a suitable acid. Representative acid addition salts include, but are not limited to acetate, adipate, alginate, citrate, aspartate, benzoate, benzenesulfonate, bisulfate, butyrate, camphorate, camphorolsulfonate, digluconate, glycerophosphate, hemisulfate, heptanoate, hexanoate, fumarate hydrochloride, hydrobromide, hydroiodide, 2-hydroxyethanesulfonate (isothionate), lactate, maleate, methanesulfonate, nicotinate, 2-naphthalenesulfonate, oxalate, palmoate, pectinate, persulfate, 3-phenylpropionate, picrate, pivalate, propionate, succinate, tartrate, thiocyanate, phosphate, glutamate, bicarbonate, p-toluenesulfonate and undecanoate. Examples of acids which may be employed to form pharmaceutically acceptable acid addition salts include inorganic acids as hydrochloric acid, hydrobromic acid, sulphuric acid and phosphoric acid and such organic acids as oxalic acid, maleic acid, succinic acid and citric acid.

Basic nitrogen-containing groups can be quaternized with agents such as, for example, lower alkyl halides including methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained.

Basic addition salts can be prepared in situ during the final isolation and purification of compounds of the present invention by reacting a carboxylic acid-containing moiety with a suitable base such as the hydroxide, carbonate or bicarbonate of a pharmaceutically acceptable metal cation or with ammonia or with an organic primary, secondary or tertiary amine. Pharmaceutically-acceptable basic addition salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

Dosage forms for topical administration of a compound of the present invention include powders, sprays, ointments and inhalants. The active compound is mixed under sterile conditions with a pharmaceutically-acceptable carrier and any needed preservatives, buffers, or propellants which may be required. Ophthalmic formulations, eye ointments, powders, and solutions are also contemplated as being within the scope of the present invention.

Actual dosage levels of active ingredients in the pharmaceutical compositions of this present invention may be varied so as to obtain an amount of the active compound(s) that is effective to achieve the desired therapeutic response for a particular patient, compositions, and mode of administration. The selected dosage level will depend upon the activity of the particular compound, the route of administration, the severity of the condition being treated, and the condition and prior medical history of the patient being treated. However, it is within the skill of the art to start doses of the compound at levels lower than required to achieve the desired therapeutic effort and to gradually increase the dosage until the desired effect is achieved.

Generally dosage levels of about 0.1 to about 1000 mg, about 0.5 to about 500 mg, about 1 to about 250 mg, about 1.5 to about 100 mg, and preferably of about 5 to about 20 mg of active compound per kilogram of body weight per day are administered orally or intravenously to a mammalian patient. If desired, the effective daily dose may be divided into multiple doses for purposes of administration, e.g., two to four separate doses per day.

The efficacy of the compounds of the present invention have been investigated and can be described by parameters, such as, for example EC50 and LC50. As used herein, the term EC50 refers to the effective concentration needed to inhibit activity by 50% in a cell based assay. The term IC50, as used herein, refers to the concentration required to inhibit protein activity in a biochemical assay by 50%. The term LD50, as used herein, refers to the compound concentration necessary to kill 50% of the cells over a defined time interval in toxicity assays.

TABLE 1
Proteins which Comprise I or A domains,
G proteins, heterotrimeric G proteins, and tubulin GTPase.

1. TIM beta/alpha-barrel (23)

| contains parallel beta-sheet barrel, closed; n = 8, S = 8; strand order 12345678 | |
|---|---|
| the first six superfamilies have similar phosphate-binding sites | |
| 1. | Triosephosphate isomerase (TIM) (1) |

| | |
|---|---|
| 1. | Triosephosphate isomerase (TIM) (12) |

| | |
|---|---|
| 2. | Ribulose-phoshate binding barrel (4) |

| 1. | Histidine biosynthesis enzymes (2) |
|---|---|
| | structural evidence for the gene duplication within the barrel |
| | fold |
| 2. | D-ribulose-5-phosphate 3-epimerase (1) |
| 3. | Orotidine 5′-monophosphate decarboxylase (OMP |
| | decarboxylase) (4) |
| 4. | Tryptophan biosynthesis enzymes (6) |

| | |
|---|---|
| 3. | Thiamin phosphate synthase (1) |

| | |
|---|---|
| 1. | Thiamin phosphate synthase (1) |

| | |
|---|---|
| 4. | FMN-linked oxidoreductases (1) |

| | |
|---|---|
| 1. | FMN-linked oxidoreductases (9) |

| | |
|---|---|
| 5. | Inosine monophosphate dehydrogenase (IMPDH) (1) |

| The phosphape moiety of substrate binds in the ‘common’ | |
|---|---|
| phosphate-binding site | |
| 1. | Inosine monophosphate dehydrogenase (IMPDH) (4) |

| | |
|---|---|
| 6. | PLP-binding barrel (2) |
| | circular permutation of the canonical fold: begins with an alpha helix |
| | and ends with a beta-strand |

| | |
|---|---|
| 1. | Alanine racemase-like, N-terminal domain (4) |
| 2. | “Hypothetical” protein yb1036c (1) |

| | |
|---|---|
| 7. | NAD(P)-linked oxidoreductase (1) |

| | |
|---|---|
| 1. | Aldo-keto reductases (NADP) (7) |
| | Common fold covers whole protein structure |

| | |
|---|---|
| 8. | (Trans)glycosidases (7) |

| | |
|---|---|
| 1. | alpha-Amylases, N-terminal domain (22) |
| | Common fold domain is interrupted by a small calcium-binding |
| | subdomain |
| | This domain is followed by an all-beta domain common to the |
| | family |
| 2. | beta-Amylase (4) |
| 3. | beta-glycanases (21) |
| | consist of a number of sequence families |
| 4. | Family 1 of glycosyl hydrolase (8) |
| 5. | Type II chitinase (9) |
| | glycosylase family 18 |
| 6. | Bacterial chitobiase (beta-N-acetylhexosaminidase), catalytic |
| | domain (1) |
| | Glycosyl hydrolase family 20 |
| 7. | Beta-D-glucan exohydrolase, N-terminal domain (1) |

| | |
|---|---|
| 9. | Metallo-dependent hydrolases (3) |
| | the beta-sheet barrel is similarly distorted and capped by a C-terminal |
| | helix has transition metal ions bound inside the barrel |

| | |
|---|---|
| 1. | Adenosine deaminase (ADA) (1) |
| 2. | alpha-subunit of urease, catalytic domain (2) |
| 3. | Phosphotriesterase-like (2) |

| | |
|---|---|
| 10. | Aldolase (4) |

| Common fold covers whole protein structure | |
|---|---|
| 1. | Class I aldolase (14) |
| | the catalytic lysine forms schiff-base intermediate with |
| | substrate |
| 2. | Class II aldolase (1) |
| | metal-dependent |
| 3. | 5-aminolaevulinate dehydratase, ALAD (porphobilinogen |
| | synthase) (3) |
| | hybrid of classes I and II aldolase |
| 4. | Class I DAHP synthetase (2) |

| | |
|---|---|
| 11. | Enolase C-terminal domain-like (2) |
| | binds metal ion (magnesium or manganese) in conserved site inside |
| | barrel |
| | N-terminal alpha + beta domain is common to this family |

| | |
|---|---|
| 1. | Enolase (2) |
| 2. | D-glucarate dehydratase-like (6) |

| | |
|---|---|
| 12. | Phosphoenolpyruvate/pyruvate domain (6) |

| | |
|---|---|
| 1. | Pyruvate kinase (5) |
| 2. | Pyruvate phosphate dikinase, C-terminal domain (1) |
| 3. | Phosphoenolpyruvate carboxylase (1) |
| 4. | Phosphoenolpyruvate mutase (1) |
| | forms a swapped dimer |
| 5. | 2-dehydro-3-deoxy-galactarate aldolase (1) |
| | forms a swapped dimer; contains a PK-type metal-binding site |
| 6. | Isocitrate lyase (2) |
| | forms a swapped dimer; elaborated with additional |
| | subdomains |

| | |
|---|---|
| 13. | Malate synthase G (1) |

| | |
|---|---|
| 1. | Malate synthase G (1) |

| | |
|---|---|
| 14. | RuBisCo, C-terminal domain (1) |

| | |
|---|---|
| 1. | RuBisCo, large subunit, C-terminal domain (6) |

N-terminal domain is alpha + beta

| | |
|---|---|
| 15. | Xylose isomerase-like (3) |
| | different families share similar but non-identical metal-binding sites |

| | |
|---|---|
| 1. | Endonuclease IV (1) |
| 2. | L-rhamnose isomerase (1) |
| 3. | Xylose isomerase (12) |

| | |
|---|---|
| 16. | Bacterial luciferase-like (3) |
| | consists of clearly related families of somewhat different folds |

| | |
|---|---|
| 1. | Bacterial luciferase (alkanal monooxygenase) (1) |
| | typical (beta/alpha)8-barrel fold |
| 2. | Non-fluorescent flavoprotein (luxF, FP390) (2) |
| | incomplete beta/alpha barrel with mixed beta-sheet of 7 |
| | strands |
| 3. | Coenzyme F420 dependent tetrahydromethanopterin reductase |
| | (1) |

| | |
|---|---|
| 17. | Quinolinic acid phosphoribosyltransferase, C-terminal domain (1) |
| | incomplete beta/alpha barrel with parallel beta-sheet of 7 strands |

| | |
|---|---|
| 1. | Quinolinic acid phosphoribosyltransferase, C-terminal domain |
| | (2) |

| | |
|---|---|
| 18. | Phosphatidylinositol-specific phospholipase C (PI-PLC) (2) |

| | |
|---|---|
| 1. | Mammalian PLC (1) |
| 2. | Bacterial PLC (2) |

| | |
|---|---|
| 19. | Cobalamin (vitamin B12)-dependent enzymes (3) |

| | |
|---|---|
| 1. | Methylmalonyl-CoA mutase, N-terminal (CoA-binding) |
| | domain (1) |
| 2. | Glutamate mutase, large subunit (1) |
| 3. | Diol dehydratase, alpha subunit (1) |

| | |
|---|---|
| 20. | tRNA-guanine transglycosylase (1) |

| | |
|---|---|
| 1. | tRNA-guanine transglycosylase (1) |

| | |
|---|---|
| 21. | Dihydropteroate synthetase-like (2) |

| | |
|---|---|
| 1. | Dihydropteroate synthetase (3) |
| 2. | Methyltetrahydrofolate: corrinoid/iron-sulfur protein |
| | methyltransferase MetR (1) |

| | |
|---|---|
| 22. | Uroporphyrinogen decarboxylase, UROD (1) |

| | |
|---|---|
| 1. | Uroporphyrinogen decarboxylase, UROD (1) |

| | |
|---|---|
| 23. | Methylenetetrahydrofolate reductase (1) |

| | |
|---|---|
| 1. | Methylenetetrahydrofolate reductase (1) |

| 2. | NAD(P)-binding Rossmann-fold domains (1) | |
|---|---|---|
| | core: 3 layers, a/b/a; parallel beta-sheet of 6 strands, order 321456 | |
| | The nucleotide-binding modes of this and the next two folds/superfamilies are | |
| | similar | |
| | 1. | NAD(P)-binding Rossmann-fold domains (8) |

| | |
|---|---|
| 1. | Alcohol/glucose dehydrogenases, C-terminal domain (9) |
| | N-terminal all-beta domain defines family |
| 2. | Tyrosine-dependent oxidoreductases (27) |
| | also known as short-chain dehydrogenases and SDR family |
| | parallel beta-sheet is extended by 7th strand, order 3214567; |
| | left-handed |
| | crossover connection between strands 6 and 7 |
| 3. | Glyceraldehyde-3-phosphate dehydrogenase-like, N-terminal |
| | domain (20) |
| | family members also share a common alpha + beta fold in |
| | C-terminal domain |
| 4. | Formate/glycerate dehydrogenases, NAD-domain (9) |
| | this domain interrupts the other domain which defines family |
| 5. | Lactate & malate dehydrogenases, N-terminal domain (16) |
| 6. | 6-phosphogluconate dehydrogenase-like, N-terminal domain |
| | (8) |
| | the beta-sheet is extended to 8 strands, order 32145678; |
| | strands 7 & 8 are antiparallel to the rest |
| | C-terminal domains also show some similarity |
| 7. | Amino-acid dehydrogenase-like, C-terminal domain (11) |
| 8. | Succinyl-CoA synthetase, alpha-chain, N-terminal |
| | (CoA-binding) domain (2) |

| 3. | FAD/NAD(P)-binding domain (1) | |
|---|---|---|
| | core: 3 layers, b/b/a; central parallel beta-sheet of 5 strands, order 32145; | |
| | top antiparallel beta-sheet of 3 strands, meander | |
| | 1. | FAD/NAD(P)-binding domain (5) |

| | |
|---|---|
| 1. | C-terminal domain of adrenodoxin reductase-like (3) |
| 2. | FAD-linked reductases, N-terminal domain (10) |
| | C-terminal domain is alpha + beta is common for the family |
| 3. | Guanine nucleotide dissociation inhibitor, GDI (1) |
| | Similar to FAD-linked reductases in both domains but does not |
| | bind FAD |
| 4. | Succinate dehydrogenase/fumarate reductase N-terminal |
| | domain (5) |
| 5. | FAD/NAD-linked reductases, N-terminal and central domains |
| | (17) |
| | duplication: both domains have similar folds and functions |
| | most members of the family contain common C-terminal |
| | alpha + beta domain |

| 4. | Nucleotide-binding domain (1) | |
|---|---|---|
| | 3 layers. a/b/a; parallel beta-sheet of 5 strands, order 32145; Rossmann-like | |
| | 1. | Nucleotide-binding domain (2) |
| | | this superfamily shares the common nucleotide-binding site with and |
| | | provides a link between the Rossmann-fold NAD(P)-binding and |
| | | FAD/NAD(P)-binding domains |

| | |
|---|---|
| 1. | N-terminal domain of adrenodoxin reductase-like (3) |
| 2. | D-amino acid oxidase, N-terminal domain (2) |
| | This family is probably related to the FAD-linked reductases |
| | and shares with them the C-terminal domain fold |

| 5. | N-terminal domain of MurD (UDP-N-acetylmuramoyl-L-alanine:D-glutamate | |
|---|---|---|
| | ligase) (1) | |
| | 3 layers: a/b/a; parallel beta-sheet of 5 strands, order 32145; incomplete | |
| | Rossmann-like fold; binds UDP group | |
| | 1. | N-terminal domain of MurD |
| | | (UDP-N-acetylmuramoyl-L-alanine:D-glutamate ligase) (1) |

| | |
|---|---|
| 1. | N-terminal domain of MurD |
| | (UDP-N-acetylmuramoyl-L-alanine:D-glutamate ligase) (1) |

| 6. | Cellulases (1) | |
|---|---|---|
| | variant of beta/alpha barrel; parallel beta-sheet barrel; closed, n = 27, S = 8; | |
| | strand order 1234567 | |
| | 1. | Cellulases (1) |

| | |
|---|---|
| 1. | Cellulases (4) |

| 7. | PFL-like glycyl radical enzymes (1) | |
|---|---|---|
| | contains. barrel, closed; n = 10, S = 10; accommodates a hairpin loop inside the | |
| | barrel | |
| | 1. | PFL-like glycyl radical enzymes (3) |
| | | duplication: the - and C-terminal halves have similar topologies |

| | |
|---|---|
| 1. | Pyruvate formate-lyase, PFL (1) |
| 2. | R1 subunit of ribonucleotide reductase, C-terminal domain (1) |
| 3. | Class III anaerobic ribonucleotide triphosphate reductase |
| | NRDD subunit (1) |

| 8. | The “swivelling” beta/beta/alpha domain (5) | |
|---|---|---|
| | 3 layers: b/b/a; the central sheet is parallel, and the other one is antiparallel; | |
| | there are some | |
| | variations in topology | |
| | this domain is thought to be mobile in all proteins known to contain it | |
| | 1. | Phosphohistidine domain (2) |
| | | contains barrel, closed, n = 7, S = 10 |

| | |
|---|---|
| 1. | Pyruvate phosphate dikinase, central domain (1) |
| 2. | N-terminal domain of enzyme I of the PEP:sugar |
| | phosphotransferase system (1) |

| | |
|---|---|
| 2. | Aconitase, C-terminal domain (1) |
| | contains mixed beta-sheet barrel, closed n = 7, S = 10 |

| | |
|---|---|
| 1. | Aconitase, C-terminal domain (2) |

| | |
|---|---|
| 3. | Carbamoyl phosphate synthetase, small subunit N-terminal domain (1) |

| | |
|---|---|
| 1. | Carbamoyl phosphate synthetase, small subunit N-terminal |
| | domain (1) |

| | |
|---|---|
| 4. | Transferrin receptor ectodomain, apical domain (1) |

| | |
|---|---|
| 1. | Transferrin receptor ectodomain, apical domain (1) |

| | |
|---|---|
| 5. | GroEL-like chaperone, apical domain (2) |

| | |
|---|---|
| 1. | GroEL (2) |
| 2. | Group II chaperonin (CCT, TRIC) (1) |

| 9. | Barstar-like (2) | |
|---|---|---|
| | 2 layers, a/b; parallel beta-sheet of 3 strands, order 123 | |
| | 1. | Barstar (barnase inhibitor) (1) |

| | |
|---|---|
| 1. | Barstar (barnase inhibitor) (1) |

| | |
|---|---|
| 2. | Ribosomal protein L32e (1) |

| | |
|---|---|
| 1. | Ribosomal protein L32e (1) |
| | contains irregular N-terminal extension to the common fold |

| 10. | Leucine-rich repeat, LRR (right-handed beta-alpha superhelix) (2) | |
|---|---|---|
| | 2 curved layers, a/b; parallel beta-sheet: order 1234 . . . N | |
| | 1. | RNI-like (3) |
| | | regular structure consisting of similar repeats |

| | |
|---|---|
| 1. | Ribonuc lease inhibitor (2) |
| 2. | Rnalp (1) |
| 3. | Cyclin A/CDK2-associated p19, Skp2 (1) |

| | |
|---|---|
| 2. | L domain-like (5) |
| | less regular structure consisting of variable repeats |

| | |
|---|---|
| 1. | Intemalin B LRR domain (1) |
| 2. | Rab geranylgeranyltransferase alpha-subunit, C-terminal |
| | domain (1) |
| 3. | mRNA export factor tap (1) |
| 4. | U2A'-like (1) |
| | duplication: consists of 5-6 partly irregular repeats |
| 5. | L1 and L2 domains of the type 1 insulin-like growth factor |
| | receptor (1) |

| 11. | Outer arm dynein light chain 1 (1) | |
|---|---|---|
| | (beta-beta-alpha)n superhelix | |
| | 1. | Outer arm dynein light chain 1 (1) |

| | |
|---|---|
| 1. | Outer arm dynein light chain 1 (1) |

| 12. | Ribosomal proteins L15p and L18e (1) | |
|---|---|---|
| | core: three turns of irregular (beta-beta-alpha)n superhelix | |
| | 1. | Ribosomal proteins L15p and L18e (1) |

| | |
|---|---|
| 1. | Ribosomal proteins LiSp and Li 8e (2) |

| 13. | SpoIIaa-like (2) | |
|---|---|---|
| | core: 4 turns of a (beta-alpha)n superhelix | |
| | 1. | C-terminal domain of phosphatidylinositol transfer protein sec14p (1) |

| | |
|---|---|
| 1. | C-terminal domain of phosphatidylinositol transfer protein |
| | sec14p (1) |
| 2. | SpoIIaa (1) |
| 1. | SpoIIaa (I) |

| 14. | ClpP/crotonase (1) | |
|---|---|---|
| | core: 4 turns of (beta-beta-alpha)n superhelix | |
| | 1. | ClpP/crotonase (3) |

| | |
|---|---|
| 1. | Clp protease, ClpP subunit (1) |
| 2. | Photosystem II Dl C-terminal processing protease, catalytic |
| | domain (1) |
| 3. | Crotonase-like (4) |

| 15. | BRCT domain (1) | |
|---|---|---|
| | 3 layers, a/b/a, core: parallel beta-sheet of 4 strands, order 2134 | |
| | 1. | BRCT domain (2) |

| | |
|---|---|
| 1. | DNA-repair protein XRCC1 (1) |
| 2. | NAD +− dependent DNA ligase, domain 4 (1) |

| 16. | beta-subunit of the lumazine synthase/riboflavin synthase complex (1) | |
|---|---|---|
| | 3 layers, a/b/a; core: parallel beta-sheet of 4 strands, order 2134 | |
| | 1. | beta-subunit of the lumazine synthase/riboflavin synthase complex (1) |

| | |
|---|---|
| 1. | beta-subunit of the lumazine synthase/riboflavin synthase |
| | complex (4) |

| 17. | Caspase-like (1) | |
|---|---|---|
| | 3 layers, a/b/a; core: parallel beta-sheet of 4 strands, order 2134 | |
| | 1. | Caspase-like (2) |
| | | heterodimeric protein folded in a single domain |

| | |
|---|---|
| 1. | Caspase (3) |
| 2. | Gingipain R (RgpB), N-terminal domain (1) |

| 18. | DNA glycosylase (1) | |
|---|---|---|
| | 3 layers, a/b/a; core: parallel beta-sheet of 4 strands, order 2134 | |
| | 1. | DNA glycosylase (2) |

| | |
|---|---|
| 1. | Uracil-DNA glycosylase (3) |
| 2. | G:T/U mismatch-specific DNA glycosylase (1) |

| 19. | Catalytic domain of malonyl-CoA ACP transacylase (1) | |
|---|---|---|
| | 3 layers, a/b/a; core: parallel beta-sheet of 4 strands, order 2134 | |
| | 1. | Catalytic domain of malonyl-CoA ACP transacylase (1) |

| | |
|---|---|
| 1. | Catalytic domain of malonyl-CoA ACP transacylase (1) |

| 20. | Initiation factor IF2/eIF5b, domain 3 (1) | |
|---|---|---|
| | 3 layers, a/b/a; core: parallel beta-sheet of 4 strands, order 2134 | |
| | 1. | Initiation factor IF2/eIF5b, domain 3 (1) |

| | |
|---|---|
| 1. | Initiation factor IF2/eIF5b, domain 3 (1) |

| 21. | Ribosomal protein L13 (1) | |
|---|---|---|
| | 3 layers, a/b/a; core: parallel beta-sheet of 4 strands, order 3214 | |
| | 1. | Ribosomal protein L13 (1) |

| | |
|---|---|
| 1. | Ribosomal protein L13 (I) |

| 22. | Ribosomal protein A (1) | |
|---|---|---|
| | 3 layers, a/b/a; core: parallel beta-sheet of 4 strands, order 1423 | |
| | 1. | Ribosomal protein IA (1) |

| | |
|---|---|
| 1. | Ribosomal protein L4 (2) |

| 23. | Flavodoxin-like (16) | |
|---|---|---|
| | 3 layers, a/b/a; parallel beta-sheet of 5 strand, order 21345 | |
| | 1. | CheY-like (3) |

| | |
|---|---|
| 1. | CheY-related (11) |
| 2. | Receiver domain of the ethylene receptor (1) |
| 3. | Negative regulator of the amidase operon AmiR (1) |

| | |
|---|---|
| 2. | Toll/Interleukin receptor TIR domain (1) |

| | |
|---|---|
| 1. | Toll/Interleukin receptor TIR domain (2) |

| | |
|---|---|
| 3. | Hypothetical protein MTH538 (1) |

| | |
|---|---|
| 1. | Hypothetical protein MTH538 (1) |

| | |
|---|---|
| 4. | Succinyl-CoA synthetase domains (1) |

| | |
|---|---|
| 1. | Succinyl-CoA synthetase domains (4) |
| | contain additional N-terminal strand “0” antiparallel to strand |
| | 2 |

| | |
|---|---|
| 5. | Flavoproteins (3) |

| | |
|---|---|
| 1. | Flavodoxin-related (8) |
| | binds FMN |
| 2. | NADPH-cytochrome p450 reductase, N-terminal domain (2) |
| 3. | Quinone reductase (4) |
| | binds FAD |

| | |
|---|---|
| 6. | Cobalamin (vitamin B12)-binding domain (1) |

| | |
|---|---|
| 1. | Cobalamin (vitamin B12)-binding domain (4) |

| | |
|---|---|
| 7. | Ornithine decarboxylase N-terminal “wing” domain (1) |

| | |
|---|---|
| 1. | Ornithine decarboxylase N-terminal “wing” domain (1) |

| | |
|---|---|
| 8. | N5-carboxyaminoimidazole ribonucleotide (N5-CAIR) mutase PurE |
| | (1) |

| | |
|---|---|
| 1. | N5-carboxyaminoimidazole ribonucleotide (N5-CAIR) mutase |
| | PurE (1) |

| | |
|---|---|
| 9. | Cutinase-like (1) |

| | |
|---|---|
| 1. | Cutinase-like (3) |
| | this family can be also classified into alpha/beta hydrolase |
| | superfamily |

| | |
|---|---|
| 10. | Esterase/acetylhydrolase (4) |

| | |
|---|---|
| 1. | Esterase (1) |
| 2. | Esterase domain of haemagglutinin-esterase-fusion |
| | glycoprotein HEF1 (1) |
| 3. | Acetylhydrolase (1) |
| 4. | Rhamnogalacturonan acetylesterase (1) |

| | |
|---|---|
| 11. | Beta-D-glucan exohydrolase, C-terminal domain (1) |

| | |
|---|---|
| 1. | Beta-D-glucan exohydrolase, C-terminal domain (1) |

| | |
|---|---|
| 12. | Formate/glycerate dehydrogenase catalytic domain-like (3) |

| | |
|---|---|
| 1. | Formate/glycerate dehydrogenases, substrate-binding domain |
| | (6) |
| | this domain is interrupted by the Rossmann-fold domain |
| 2. | L-alanine dehydrogenase (1) |
| 3. | S-adenosylhomocystein hydrolase (2) |

| | |
|---|---|
| 13. | Type II 3-dehydroquinate dehydratase (1) |

| | |
|---|---|
| 1. | Type II 3-dehydroquinate dehydratase (2) |

| | |
|---|---|
| 14. | Nucleoside 2-deoxyribosyltransferase (1) |

| | |
|---|---|
| 1. | Nucleoside 2-deoxyribosyltransferase (1) |

| | |
|---|---|
| 15. | Ribosomal protein S2 (1) |
| | fold elaborated with additional structures |

| | |
|---|---|
| 1. | Ribosomal protein S2 (1) |

| | |
|---|---|
| 16. | Class I glutamine amidotransferase-like (4) |
| | conserved positions of the oxyanion hole and catalytic nucleophile; |
| | different constituent families contain different additional structures |

| | |
|---|---|
| 1. | Class I glutamine amidotransferases (GAT) (3) |
| | contains a catalytic Cys-His-Glu triad |
| 2. | Intracellular protease (1) |
| | contains a catalytic Cys-His-Glu triad that differs from the |
| | class I GAT triad |
| 3. | Catalase, C-terminal domain (1) |
| 4. | Aspartyl dipeptidase PepE (1) |
| | probable circular permutation in the common core; contains a |
| | catalytic Ser-His-Glu triad |

| 24. | Methylglyoxal synthase-like (1) | |
|---|---|---|
| | 3 layers, a/b/a; parallel beta-sheet of 5 strands, order 32145 | |
| | 1. | Methylglyoxal synthase-like (2) |
| | | contains a common phosphate-binding site |

| | |
|---|---|
| 1. | Carbamoyl phosphate synthetase, large subunit allosteric, |
| | C-terminal domain (1) |
| 2. | Methylglyoxal synthase, MgsA (1) |

| 25. | Ferredoxin reductase-like, C-terminal NADP-linked domain (1) | |
|---|---|---|
| | 3 layers, a/b/a; parallel beta-sheet of 5 strands, order 32145 | |
| | 1. | Ferredoxin reductase-like, C-terminal NADP-linked domain (5) |
| | | binds NADP diferently than classical Rossmann-fold |
| | | N-terminal FAD-linked domain contains (6, 10) barrel |

| | |
|---|---|
| 1. | Reductases (10) |
| 2. | Phthalate dioxygenase reductase (1) |
| | contains additional 2Fe-2S ferredoxin domain |
| 3 | Dihydroorotate dehydrogenase B, PyrK subunit (1) |
| | contains 2Fe-2S cluster in the C-terminal extension |
| 4. | NADPH-cytochrome p450 reductase-like (2) |
| 5. | Flavohemoglobin, C-terminal domain (1) |
| | contains additional globin domain |

| 26. | Adenine nucleotide alpha hydrolase-like (3) | |
|---|---|---|
| | core: 3 layers, a/b/a; parallel beta-sheet of 5 strands, order 32145 | |
| | 1. | Nucleotidylyl transferase (3) |

| | |
|---|---|
| 1. | Class I aminoacyl-tRNA synthetases (RS), catalytic domain |
| | (10) |
| | contains a conserved all-alpha subdomain at the C-terminal |
| | extension |
| 2. | Cytidylyltransferase (1) |
| 3. | Adenylyltransferase (2) |

| | |
|---|---|
| 2. | Adenine nucleotide alpha hydrolases (2) |

| | |
|---|---|
| 1. | N-type ATP pyrophosphatases (3) |
| 2. | Phosphoadenylyl sulphate (PAPS) reductase (1) |

| | |
|---|---|
| 3. | UDP-glucose dehydrogenase (UDPGDH), C-terminal (UDP-binding) |
| | domain (1) |

| | |
|---|---|
| 1. | UDP-glucose dehydrogenase (UDPGDH), C-terminal |
| | (UDP-binding) domain (1) |

| 27. | Pyrimidine nucleoside phosphorylase central domain (1) | |
|---|---|---|
| | 3 layers: a/b/a; parallel beta-sheet of 5 strands, order 32145; Rossmann-like | |
| | 1. | Pyrimidine nucleoside phosphorylase central domain (1) |

| | |
|---|---|
| 1. | Pyrimidine nucleoside phosphorylase central domain (2) |

| 28. | N-terminal domain of DNA photolyase (1) | |
|---|---|---|
| | 3 layers: a/b/a; parallel beta-sheet of 5 strands, order 32145; Rossmann-like | |
| | 1. | N-terminal domain of DNA photolyase (1) |

| | |
|---|---|
| 1. | N-terminal domain of DNA photolyase (2) |

| 29. | ETFP adenine nucleotide-binding domain-like (1) | |
|---|---|---|
| | 3 layers: a/b/a, core: parallel beta-sheet of 5 strands, order 32145 | |
| | 1. | ETFP adenine nucleotide-binding domain-like (2) |

| | |
|---|---|
| 1. | Electron transfer flavoprotein, ETFP (2) |
| | contains additional strands on both edges of the core sheet |
| 2. | “Hypothetical” protein MJ0577 (1) |

| 30. | Biotin carboxylase N-terminal domain-like (1) | |
|---|---|---|
| | 3 layers: a/b/a; parallel or mixed beta-sheet of 4 to 6 strands | |
| | possible rudiment form of Rossmann-fold domain | |
| | 1. | Biotin carboxylase N-terminal domain-like (5) |
| | | superfamily defined by the common ATP-binding domain that follows |
| | | this one |
| | 1. | Biotin carboxylase/Carbamoyl phosphate synthetase (5) |
| | 2. | D-Alanine ligase N-terminal domain (2) |
| | 3. | Prokaryotic glutathione synthetase, N-terminal domain (1) |
| | 4. | Eukaryotic glutathione synthetase (1) |
| | | circularly permuted version ofprokaryotic enzyme |
| | 5. | Synapsin Ia domain (1) |
| 31. | DHS-like NAD/FAD-binding domain (1) | |
| | 3 layers: a/b/a; parallel beta-sheet of 6 strands, order 321456; Rossmann-like | |
| | 1. | DHS-like NAD/FAD-binding domain (4) |
| | | binds cofactor molecules in the opposite direction than classical |
| | | Rossman fold |

| | |
|---|---|
| 1. | Deoxyhypusine synthase, DHS (1) |
| 2. | C-terminal domain of the electron transfer flavoprotein alpha |
| | subunit (2) |
| | lacks strand 3; shares the FAD-binding mode with the pyruvate |
| | oxidase domain |
| 3. | Pyruvate oxidase and decarboxylase, middle domain (5) |
| | N-terminal domain is Pyr module, and C-terminal domain is |
| | PP module of thiam in diphosphate-binding fold |
| 4. | Transhydrogenase domain III (dIII) (3) |
| | binds NADP, shares with the pyruvate oxidase FAD-binding |
| | domain a common ADP-binding mode |

| 32. | Tubulin, GTPase domain (1) | |
|---|---|---|
| | 3 layers: a/b/a; parallel beta-sheet of 6 strands, order 321456 | |
| | 1. | Tubulin, GTPase domain (1) |

| | |
|---|---|
| 1. | Tubulin, GTPase domain (3) |

| 33. | Cysteine hydrolase (1) | |
|---|---|---|
| | 3 layers: a/b/a; parallel beta-sheet of 6 strands, order 321456 | |
| | 1. | Cysteine hydrolase (2) |

| | |
|---|---|
| 1. | N-carbamoylsarcosine amidohydrolase (1) |
| 2. | YcaC (1) |

| 34. | Halotolerance protein Hal3 (1) | |
|---|---|---|
| | 3 layers: a/b/a; parallel beta-sheet of 6 strands, order 321456 | |
| | 1. | Halotolerance protein Hal3 (1) |

| | |
|---|---|
| 1. | Halotolerance protein Hal3 (1) |

| 35. | Glucosamine 6-phosphate deaminase/isomerase (1) | |
|---|---|---|
| | 3 layers: a/b/a; parallel beta-sheet of 6 strands, order 324561 | |
| | 1. | Glucosamine 6-phosphate deaminase/isomerase (1) |

| | |
|---|---|
| 1. | Glucosamine 6-phosphate deaminase/isomerase (2) |

| 36. | Thiamin diphosphate-binding fold (THDP-binding) (1) | |
|---|---|---|
| | 3 layers: a/b/a; parallel beta-sheet of 6 strands, order 213465 | |
| | 1. | Thiamin diphosphate-binding fold (THDP-binding) (4) |
| | | both pyridine (Pyr)- and pyrophosphate (PP)-binding modules have |
| | | this fold |
| | | conserved core consists of two Pyr and two PP-modules and binds two |
| | | coenzyme molecules |

| | |
|---|---|
| 1. | Pyruvate oxidase and decarboxylase (5) |
| | Pyr module is N-terminal domain, PP module is C-terminal |
| | domain |
| | Rossmann-like domain is between them |
| 2. | Transketolase, TK (1) |
| 3. | Branched-chain alpha-keto acid dehydrogenase (2) |
| | parent family to TK and PFOR |
| | heterodimeric protein related to TK; alpha-subunit is the PP |
| | module and the N-terminal domain of beta-subunit is the Pyr |
| | module |
| 4. | Pyruvate-ferredoxin oxidoreductase, PFOR, domains I and VI |
| | (1) |
| | domains VI, I and II are arranged in the same way as the TK N, |
| | M and C domains |

| 37. | P-loop containing nucleotide triphosphate hydrolases (1) | |
|---|---|---|
| | 3 layers; a/b/a, parallel or mixed beta-sheets of variable sizes | |
| | 1. | P-loop containing nucleotide triphosphate hydrolases (14) |
| | | division into families based on beta-sheet topologies |

| | |
|---|---|
| 1. | Nucleotide and nucleoside kinases (16) |
| | parallel beta-sheet of 5 strands, order 23145 |
| 2. | Shikimate kinase (1) |
| | similar to the nucleotide/nucleoside kinases but acts on |
| | different substrate |
| 3. | Chloramphenicol phosphotransferase (1) |
| | similar to the nucleotide/nucleoside kinases but acts on |
| | different substrate |
| 4. | Adenosine-5′phosphosulfate kinase (APS kinase) (1) |
| 5. | PAPS sulfotransferase (4) |
| | similar to the nucleotide/nucleoside kinases but transfer |
| | sulphate group |
| 6. | Phosphoribulokinase/pantothenate kinase (2) |
| 7. | 6-phosphofructo-2-kinase/fructose-2,6-bisphosphatase, kinase |
| | domain (1) |
| 8. | G proteins (28) |
| | core: mixed beta-sheet of 6 strands, order 231456; strand 2 is |
| | antiparallel to the rest |
| 9. | Motor proteins (7) |
| 10. | Nitrogenase iron protein-like (10) |
| | core: parallel beta-sheet of 7 strands; order 3241567 |
| 11. | RecA protein-like (ATPase-domain) (9) |
| | core: mixed beta-sheet of 8 strands, order 3245 1678, strand 7 |
| | is antiparallel to the rest |
| 12. | ABC transporter ATPase domain-like (7) |
| | there are two additional subdomains inserted into the central |
| | core that has a RecA-like topology |
| 13. | Extended AAA-ATPase domain (13) |
| | fold is similar to that of RecA, but lacks the last two strands, |
| | followed by a family-specific all-alpha Arg-finger domain |
| 14. | RNA helicase (1) |
| | duplication: consists of two similar domains, one binds NTP |
| | and the other binds RNA; also contains an all-alpha |
| | subdomain in the C-terminal extension |

| 38. | Fructose permease, subunit IIb (1) | |
|---|---|---|
| | 3 layers: a/b/a, parallel beta-sheet of 6 strands, order 324156 | |
| | 1. | Fructose permease, subunit IIb (1) |

| | |
|---|---|
| 1. | Fructose permease, subunit IIb (1) |

| 39. | Nicotinate mononucleotide: 5,6-dimethylbenzimidazole | |
|---|---|---|
| | phosphoribosyltransferase (CobT) (1) | |
| | 3 layers: a/b/a, parallel beta-sheet of 7 strands, order 3214567 | |
| | 1. | Nicotinate mononucleotide: 5,6-dimethylbenzimidazole |
| | | phosphoribosyltransferase (CobT) (1) |
| | 1. | Nicotinate mononucleotide:5,6-dimethylbenzimidazole |
| | | phosphoribosyltransferase (CobT) (1) |
| 40. | Methylesterase CheB, C-terminal domain (1) | |
| | 3 layers. a/b/a, parallel beta-sheet of 7 strands, order 3421567 | |
| | 1. | Methylesterase CheB, C-terminal domain (1) |

| | |
|---|---|
| 1. | Methylesterase CheB, C-terminal domain (1) |

| 41. | Subtilisin-like (1) | |
|---|---|---|
| | 3 layers. a/b/a, parallel beta-sheet of 7 strands, order 2314567; left-handed | |
| | crossover connection between strands 2 & 3 | |
| | 1. | Subtilisin-like (2) |

| | |
|---|---|
| 1. | Subtilases (12) |
| 2. | Serine-carboxyl proteinase PSCP (1) |
| | elaborated with additional structures |

| 42. | Arginase/deacetylase (1) | |
|---|---|---|
| | 3 layers: a/b/a, parallel beta-sheet of 8 strands, order 21387456 | |
| | 1. | Arginase/deacetylase (2) |

| | |
|---|---|
| 1. | Arginase (2) |
| 2. | Histone deacetylase, HDAC (1) |

| 43. | CoA-dependent acyltransferases (1) | |
|---|---|---|
| | core. 2 layers, a/b; mixed beta-sheet of 6 strands, order 324561; strands 3 & | |
| | 6 are antiparallel to the rest | |
| | 1. | CoA-dependent acyltransferases (1) |

| | |
|---|---|
| 1. | CoA-dependent acyltransferases (5) |

| 44. | Phosphotyrosine protein phosphatases I-like (2) | |
|---|---|---|
| | 3 layers: a/b/a; parallel beta-sheet of 4 strands, order 2134 | |
| | 1. | Phosphotyrosine protein phosphatases I (1) |
| | | share the common active site structure with the family II |

| | |
|---|---|
| 1. | Low-molecular-weight phosphotyrosine protein phosphatases (3) |

| | |
|---|---|
| 2. | Enzyme IIB-cellobiose (I) |

| | |
|---|---|
| 1. | Enzyme IIB-cellobiose (1) |

| 45. | (Phosphotyrosine protein) phosphatases II (1) | |
|---|---|---|
| | core: 3 layers, a/b/a; parallel beta-sheet of 4 strands, order 1432 | |
| | 1. | (Phosphotyrosine protein) phosphatases II (3) |
| | | share with the family I the common active site structure with a |
| | | circularly permuted topology |

| | |
|---|---|
| 1. | Dual-specificity phosphatases (2) |
| 2. | Higher-molecular-weight phosphotyrosine protein phosphatases (8) |
| | have an extension to the beta-sheet of 3 antiparallel strands |
| | before strand 4 |
| 3. | Phoshphoinositide phosphatase Pten (Pten tumor suppressor), |
| | N-terminal domain (1) |

| 46. | Rhodanese/Cell cycle control phosphatase (1) | |
|---|---|---|
| | 3 layers; a/b/a; parallel beta-sheet of 5 strands, order 32451 | |
| | 1. | Rhodanese/Cell cycle control phosphatase (2) |
| | | the active site structure is similar to those of the families I and II |
| | | protein phosphatases; the topology can be related by a diferent |
| | | circular permutation to the family I topology |

| | |
|---|---|
| 1. | Cell cycle control phosphatase, catalytic domain (2) |
| 2. | Sulfurtransferase (rhodanese) (2) |
| | duplication, consists of two domains of this fold |

| 47. | Thioredoxin fold (3) | |
|---|---|---|
| | core: 3 layers, a/b/a; mixed beta-sheet of 4 strands, order 4312; strand 3 is | |
| | antiparallel to the rest | |
| | 1. | Thioredoxin-like (10) |

| | |
|---|---|
| 1. | Thioltransferase (12) |
| 2. | PDI-like (3) |
| | duplication: contains two tandem repeats of this fold |
| 3. | Calsequestrin (1) |
| | duplication: contains three tandem repeats of this fold |
| 4. | Disulphide-bond formation facilitator (DSBA) (2) |
| 5. | Glutathione S-transferases, N-terminal domain (23) |
| 6. | Phosducin (2) |
| 7. | Endoplasmic reticulum protein ERP29, N-domain (1) |
| 8. | spliceosomal protein U5-l5Kd (1) |
| 9. | Disulfide bond isomerase, DsbC, C-terminal domain (1) |
| | elaborated common fold |
| 10. | Glutathione peroxidase-like (6) |

| | |
|---|---|
| 2. | RNA 3′-terminal phosphate cyclase, RPTC, insert domain (1) |

| | |
|---|---|
| 1. | RNA 3′-terminal phosphate cyclase, RPTC, insert domain (1) |

| | |
|---|---|
| 3. | Thioredoxin-like 2Fe-2S ferredoxin (1) |

| | |
|---|---|
| 1. | Thioredoxin-like 2Fe-2S ferredoxin (1) |

| 48. | Transketolase C-terminal domain-like (1) | |
|---|---|---|
| | 3 layers: a/b/a; mixed beta-sheet of 5 strands, order 13245, strand 1 is | |
| | antiparallel to the rest | |
| | 1. | Transketolase C-terminal domain-like (3) |

| | |
|---|---|
| 1. | Transketolase (1) |
| 2. | Branched-chain alpha-keto acid dehydrogenase beta-subunit, |
| | C-domain (2) |
| 3. | Pyruvate-ferredoxin oxidoreductase, PFOR, domain II (1) |

| 49. | Pyruvate kinase C-terminal domain-like (2) | |
|---|---|---|
| | 3 layers: a/b/a; mixed beta-sheet of 5 strands, order 32145, strand 5 is | |
| | antiparallel to the rest | |
| | 1. | Pyruvate kinase, C-terminal domain (1) |

| | |
|---|---|
| 1. | Pyruvate kinase, C-terminal domain (5) |

| | |
|---|---|
| 2. | ATP syntase (F1-ATPase), gamma subunit (1) |
| | contains an antiparallel coiled coil formed by - anb C-terminal |
| | extensions to the common fold |

| | |
|---|---|
| 1. | ATP syntase (F1-ATPase), gamma subunit (2) |

| 50. | Leucine aminopeptidase, N-terminal domain (1) | |
|---|---|---|
| | 3 layers: a/b/a; mixed beta-sheet of 5 strands, order 23145; strand 2 is | |
| | antiparallel to the rest | |
| | 1. | Leucine aminopeptidase, N-terminal domain (1) |

| | |
|---|---|
| 1. | Leucine aminopeptidase, N-terminal domain (1) |

| 51. | Anticodon-binding domain-like (4) | |
|---|---|---|
| | 3 layers: a/b/a; mixed beta-sheet of five strands, order 21345; strand 4 is | |
| | antiparallel to the rest | |
| | 1. | Anticodon-binding domain of Class II aaRS (1) |

| | |
|---|---|
| 1. | Anticodon-binding domain of Class II aaRS (5) |

| | |
|---|---|
| 2. | TolB, N-terminal domain (1) |

| | |
|---|---|
| 1. | TolB, N-terminal domain (1) |

| | |
|---|---|
| 3. | Diol dehydratase, beta subunit (1) |

| | |
|---|---|
| 1. | Diol dehydratase, beta subunit (1) |
| | contains additional structures in the C-terminal extension |

| | |
|---|---|
| 4. | Maf/Ham1 (2) |
| | elaborated with additional structures inserted in the common fold |

| | |
|---|---|
| 1. | Ham1 (1) |
| 2. | Maf protein (1) |

| 52. | Restriction endonuclease-like (3) | |
|---|---|---|
| | core: 3 layers, a/b/a; mixed beta-sheet of 5 strands, order 12345; strands 2 &, | |
| | in some families, 5 are antiparallel to the rest | |
| | 1. | Restriction endonuclease-like (17) |

| | |
|---|---|
| 1. | Restriction endonuclease EcoRI (1) |
| 2. | Restriction endonuclease EcoRV (1) |
| 3. | Restriction endonuclease BamHI (1) |
| 4. | Restriction endonuclease BglI (1) |
| 5. | Restriction endonuclease BglII (1) |
| 6. | Restriction endonuclease PvuII (1) |
| 7. | Restriction endonuclease Cfr0I (1) |
| 8. | Restriction endonuclease MunI (1) |
| 9. | Restriction endonuclease NaeI (1) |
| 10. | Restriction endonuclease NgoIV (1) |
| 11. | Restriction endonuclease BsobI (1) |
| 12. | Restriction endonuclease FokI, C-terminal (catalytic) domain (1) |
| 13. | lambda exonuclease (1) |
| 14. | DNA mismatch repair protein MutH from (1) |
| 15. | Very short patch repair (VSR) endonuclease (1) |
| 16. | TnsA endonuclease, N-terminal domain (1) |
| 17. | Holliday junction resolvase (Endonuclease I) (1) |

| | |
|---|---|
| 2. | tRNA splicing endonuclease, C-terminal domain (1) |

| | |
|---|---|
| 1. | tRNA splicing endonuclease, C-terminal domain (1) |

| | |
|---|---|
| 3. | Eukaryotic RPB5 N-terminal domain (1) |

| | |
|---|---|
| 1. | Eukaryotic RPB5 N-terminal domain (1) |

| 53. | Resolvase-like (2) | |
|---|---|---|
| | Core: 3 layers: a/b/a; mixed beta-sheet of 5 strands, order 21345; strand 5 is | |
| | antiparallel to the rest | |
| | 1. | Resolvase-like (2) |

| | |
|---|---|
| 1. | gamma, delta resolvase, large fragment (1) |
| 2. | 5′ to 3′ exonuclease (5) |
| | contains additional strand and alpha-helical arch; strand |
| | order 321456; strand 6 is antiparallel to the rest |

| | |
|---|---|
| 2. | beta-carbonic anhydrase (1) |

| | |
|---|---|
| 1. | beta-carbonic anhydrase (2) |

| 54. | IIA domain of mannose transporter, IIA-Man (1) | |
|---|---|---|
| | 3 layers: a/b/a; mixed beta-sheet of 5 strands, order 21345; strand 5 is | |
| | antiparallel to the rest | |
| | 1. | IIA domain of mannose transporter, IIA-Man (1) |
| | | active dimer is formed by strand 5 swapping |

| | |
|---|---|
| 1. | IIA domain of mannose transporter, IIA-Man (1) |

| 55. | Ribonuclease H-like motif (7) | |
|---|---|---|
| | 3 layers: a/b/a; mixed beta-sheet of 5 strands, order 32145, strand 2 is | |
| | antiparallel to the rest | |
| | 1. | Actin-like ATPase domain (4) |
| | | duplication contains two domains of this fold |

| | |
|---|---|
| 1. | Actin/HSP70 (8) |
| 2. | Acetate kinase (1) |
| 3. | Hexokinase (3) |
| 4. | Glycerol kinase (1) |

| | |
|---|---|
| 2. | Creatinase/prolidase N-terminal domain (1) |

| | |
|---|---|
| 1. | Creatinase/prolidase N-terminal domain (2) |

| | |
|---|---|
| 3. | Ribonuclease H-like (6) |
| | consists of one domain of this fold |

| | |
|---|---|
| 1. | Ribonuclease H (4) |
| 2. | Retroviral integrase, catalytic domain (3) |
| 3. | mu transposase, core domain (1) |
| 4. | Transposase inhibitor (Tn5 transposase) (1) |
| 5. | DnaQ-like 3′-5′ exonuclease (11) |
| 6. | RuvC resolvase (1) |

| | |
|---|---|
| 4. | Translational machinery components (2) |

| | |
|---|---|
| 1. | Ribosomal protein L18 and S11 (2) |
| 2. | Middle domain of eukaryotic peptide chain release factor |
| | subunit 1, ERF1 (1) |

| | |
|---|---|
| 5. | Hypothetical protein MTH1175 (1) |

| | |
|---|---|
| 1. | Hypothetical protein MTH1175 (1) |

| | |
|---|---|
| 6. | DNA repair protein MutS, domain II (1) |

| | |
|---|---|
| 1. | DNA repair protein MutS, domain II (2) |

| | |
|---|---|
| 7. | Methylated DNA-protein cysteine methyltransferase domain (1) |

| | |
|---|---|
| 1. | Methylated DNA-protein cysteine methyltransferase domain (3) |

| 56. | Phosphorylase/hydrolase-like (6) | |
|---|---|---|
| | core: 3 layers, a/b/a; mixed sheet of 5 strands: order 21354: strand 4 is | |
| | antiparallel to the rest; contains crossover loops | |
| | 1. | Hydrogenase maturating endopeptidase HybD (1) |
| | | the fold coincides with the consensus core structure |

| | |
|---|---|
| 1. | Hydrogenase maturating endopeptidase HybD (1) |

| | |
|---|---|
| 2. | Purine and uridine phosphorylases (1) |
| | complex architecture; contains mixed beta-sheet of 8 strands, order |
| | 23415867, strands 3, 6 & 7 are antiparallel to the rest; and barrel, |
| | closed; n = 5, S = 8 |

| | |
|---|---|
| 1. | Purine and uridine phosphorylases (6) |

| | |
|---|---|
| 3. | Peptidyl-tRNA hydrolase (1) |

| | |
|---|---|
| 1. | Peptidyl-tRNA hydrolase (1) |

| | |
|---|---|
| 4. | Pyrrolidone carboxyl peptidase (pyroglutamate aminopeptidase) (1) |

| | |
|---|---|
| 1. | Pyrrolidone carboxyl peptidase (pyroglutamate |
| | aminopeptidase) (2) |

| | |
|---|---|
| 5. | Zn-dependent exopeptidases (5) |
| | core: mixed beta-sheet of 8 strands, order 12435867; strands 2, 6 & 7 |
| | are antiparallel to the rest |

| | |
|---|---|
| 1. | Pancreatic carboxypeptidases (6) |
| 2. | Carboxypeptidase T (1) |
| 3. | Leucine aminopeptidase, C-terminal domain (1) |
| 4. | Bacterial exopeptidases (3) |
| 5. | Transferrin receptor ectodomain, protease-like domain (1) |

| | |
|---|---|
| 6. | LigB subunit of an aromatic-ring-opening dioxygenase LigAB (1) |
| | circular permutation of the common fold, most similar to the PNP fold |

| | |
|---|---|
| 1. | LigB subunit of an aromatic-ring-opening dioxygenase LigAB (1) |

| 57. | Molybdenumm cofactor biosynthesis protein MogA (1) | |
|---|---|---|
| | 3 layers: a/b/a; mixed beta-sheet of 5 strands; order: 21354, strand 5 is | |
| | antiparallel to the rest; permutation of the Phosphorylase/hydrolase-like fold | |
| | 1. | Molybdenumm cofactor biosynthesis protein MogA (1) |

| | |
|---|---|
| 1. | Molybdenumm cofactor biosynthesis protein MogA (1) |

| 58. | Amino acid dehydrogenase-like, N-terminal domain (1) | |
|---|---|---|
| | 3 layers: a/b/a; mixed beta-sheet of 5 strands; 12435, strand 2 is antiparallel | |
| | to the rest | |
| | 1. | Amino acid dehydrogenase-like, N-terminal domain (3) |

| | |
|---|---|
| 1. | Amino acid dehydrogenases (7) |
| | dimerisation domain |
| 2. | Tetrahydrofolate dehydrogenase/cyclohydrolase (3) |
| 3. | Mitochondrial NAD(P)-dependent malic enzyme (1) |
| | this domain is decorated with additional structures; includes |
| | N-terminal additional subdomains |

| 59. | Glutamate ligase domain (1) | |
|---|---|---|
| | 3 layers: a/b/a; mixed beta-sheet of 6 strands, order 126345, strand 1 is | |
| | antiparallel to the rest | |
| | 1. | Glutamate ligase domain (2) |

| | |
|---|---|
| 1. | MurD/MurF C-terminal domain (2) |
| 2. | Folylpolyglutamate synthetase, C-terminal domain (1) |

| 60. | Phosphoglycerate mutase-like (1) | |
|---|---|---|
| | core: 3 layers, a/b/a; mixed beta-sheet of 6 strands, order 324156; strand 5 is | |
| | antiparallel to the rest | |
| | 1. | Phosphoglycerate mutase-like (4) |

| | |
|---|---|
| 1. | Phosphoglycerate mutase (1) |
| 2. | Acid phosphatase (2) |
| 3. | Phytase (myo-inositol-hexakisphosphate-3-phosphohydrolase) (3) |
| 4. | 6-phosphofructo-2-kinase/fructose-2,6-bisphosphatase, |
| | phosphatase domain (1) |

| 61. | PRTase-like (1) | |
|---|---|---|
| | core: 3 layers, a/b/a; mixed beta-sheet of 6 strands, order 321456; strand 3 is | |
| | antiparallel to the rest | |
| | 1. | PRTase-like (2) |

| | |
|---|---|
| 1. | Phosphoribosyltransferases (PRTases) (14) |
| 2. | Phosphoribosylpyrophosphate synthetase (1) |
| | duplication: consists of two domains of this fold |

| 62. | Integrin A (or I) domain (1) | |
|---|---|---|
| | core: 3 layers, a/b/a; mixed beta-sheet of 6 strands, order 321456: strand 3 is | |
| | antiparallel to the rest | |
| | 1. | Integrin A (or I) domain (1) |

| | |
|---|---|
| 1. | Integrin A (or I) domain (7) |

| 63. | Glutaconate-CoA transferase subunits (1) | |
|---|---|---|
| | core. 3 layers: a/b/a; parallel or mixed b-sheet of 6 strands, order 432156; | |
| | part of sheet is folded upon itself and forms a barrel-like structure | |
| | 1. | Glutaconate-CoA transferase subunits (1) |

| | |
|---|---|
| 1. | Glutaconate-CoA transferase subunits (2) |

| 64. | Pyruvate-ferredoxin oxidoreductase, PFOR, domain III (1) | |
|---|---|---|
| | 3 layers. a/b/a, mixed beta-sheet of 6 strands, order 231456; strand 3 is | |
| | antiparallel to the rest | |
| | 1. | Pyruvate-ferredoxin oxidoreductase, PFOR, domain III (1) |

| | |
|---|---|
| 1. | Pyruvate-ferredoxin oxidoreductase, PFOR, domain III (1) |

| 65. | Formyltransferase (1) | |
|---|---|---|
| | 3 layers: a/b/a; mixed beta-sheet of 7 strands, order 3214567; strand 6 is | |
| | antiparallel to the rest | |
| | 1. | Formyltransferase (1) |

| | |
|---|---|
| 1. | Formyltransferase (2) |

| 66. | S-adenosyl-L-methionine-dependent methyltransferases (1) | |
|---|---|---|
| | core: 3 layers, a/b/a; mixed beta-sheet of 7 strands, order 32145 76, strand 7 | |
| | is antiparallel to the rest | |
| | 1. | S-adenosyl-L-methionine-dependent methyltransferases (11) |

| | |
|---|---|
| 1. | Catechol O-methyltransferase, COMT (1) |
| 2. | RNA methyltransferase FtsJ (1) |
| 3. | Fibrillarin homologue (1) |
| 4. | Hypothetical protein MJ0882 (1) |
| 5. | Glycine N-methyltransferase (1) |
| 6. | Arginine methyltransferase, HMT 1 (1) |
| | lacks the last two strands of the common fold replaced with a |
| | beta-sandwich oligomerisation subdomain |
| 7. | Protein-L-isoaspartate O-methyltransferase (1) |
| | another C-terminal variation of the common fold with |
| | additional alpha + beta subdomain |
| 8. | Chemotaxis receptor methyltransferase CheR, C-terminal |
| | domain (1) |
| | contains additional N-terminal all-alpha domain, res. 11-91 |
| 9. | RNA methylases (3) |
| 10. | DNA methylases (5) |
| 11. | Type II DNA methylase (2) |
| | circularly permuted version of the common fold |

| 67. | PLP-dependent transferases (1) | |
|---|---|---|
| | main domain: 3 layers: a/b/a, mixed beta-sheet of 7 strands, order 3245671; | |
| | strand 7 is antiparallel to the rest | |
| | 1. | PLP-dependent transferases (5) |

| | |
|---|---|
| 1. | AAT-like (9) |
| 2. | Beta-eliminating lyases (2) |
| 3. | Cystathionine synthase-like (8) |
| 4. | omega-Amino acid:pyruvate aminotransferase-like (15) |
| 5. | Ornithine decarboxylase major domain (1) |

| 68. | Nucleotide-diphospho-sugar transferases (1) | |
|---|---|---|
| | 3 layers: a/b/a; mixed beta-sheet of 7 strands, order 3214657; strand 6 is | |
| | antiparallel to the rest | |
| | 1. | Nucleotide-diphospho-sugar transferases (8) |

| | |
|---|---|
| 1. | Spore coat polysaccharide biosynthesis protein SpsA (1) |
| 2. | beta 1,4 galactosyltransferase (b4GalTl) (1) |
| 3. | CMP acylneuraminate synthetase (1) |
| 4. | Galactosyltransferase LgtC (1) |
| 5 | N-acetylglucosamine 1-phosphate uridyltransferase GlmU, |
| | N-terminal domain (1) |
| 6. | glucose-1-phosphate thymidylyltransferase RmlA (1) |
| 7. | 1,3-Glucuronyltransferase I (glcAT-I) (1) |
| 8. | Molybdenum cofactor biosynthesis protein MobA (1) |

| 69. | alpha/beta-Hydrolases (1) | |
|---|---|---|
| | core: 3 layers, a/b/a; mixed beta-sheet of 8 strands, order 12435678, strand 2 | |
| | is antiparallel to the rest | |
| | 1. | alpha/beta-Hydrolases (20) |
| | | many members have left-handed crossover connection between strand |
| | | 8 and additional strand 9 |

| | |
|---|---|
| 1. | Acetylcholinesterase-like (8) |
| 2. | Carboxylesterase (2) |
| 3. | Mycobacterial antigens (2) |
| 4. | Prolyl oligopeptidase, C-terminal domain (1) |
| 5. | Serine carboxypeptidase (4) |
| 6. | Gastric lipase (1) |
| 7. | Proline iminopeptidase (2) |
| 8. | Haloalkane dehalogenase (3) |
| 9. | Dienelactone hydrolase (2) |
| 10. | Carbon-carbon bond hydrolase (1) |
| 11. | Epoxide hydrolase (3) |
| 12. | Haloperoxidase (5) |
| 13. | Thioesterases (2) |
| 14. | Carboxylesterase/thioesterase 1 (2) |
| 15. | A novel bacterial esterase (1) |
| 16. | Lipase (1) |
| 17. | Fungal lipases (9) |
| 18. | Bacterial lipase (5) |
| 19. | Pancreatic lipase, N-terminal domain (6) |
| 20. | Hydroxynitrile lyase (2) |

| 70. | Nucleoside hydrolase (1) | |
|---|---|---|
| | core: 3 layers, a/b/a; mixed beta-sheet of 8 strands, order 32145687; strand 7 | |
| | is antiparallel to the rest | |
| | 1. | Nucleosidehydrolase (1) |

| | |
|---|---|
| 1. | Nucleoside hydrolase (2) |

| 71. | Dihydrofolate reductases (1) | |
|---|---|---|
| | 3 layers: a/b/a; mixed beta-sheet of 8 strands, order 34251687; strand 8 is | |
| | antiparallel to the rest | |
| | 1. | Dihydrofolate reductases (1) |

| | |
|---|---|
| 1. | Dihydrofolate reductases (10) |

| 72. | Ribokinase-like (2) | |
|---|---|---|
| | core: 3 layers: a/b/a; mixed beta-sheet of 8 strands, order 21345678, strand 7 | |
| | is antiparallel to the rest | |
| | potential superfamily: members of this fold have similar functions but different | |
| | ATP-binding sites | |
| | 1. | Ribokinase-like (2) |
| | | has extra strand located between strands 2 and 3 |

| | |
|---|---|
| 1. | Ribokinase-like (3) |
| 2. | Hydroxyethylthiazole kinase (thz kinase) (1) |

| | |
|---|---|
| 2. | MurD-like peptide ligases, catalytic domain (2) |
| | has extra strand located between strands 1 and 2 |

| | |
|---|---|
| 1. | MurD/MurF (2) |
| 2. | Folylpolyglutamate synthetase (1) |

| 73. | Carbamate kinase-like (1) | |
|---|---|---|
| | 3 layers: a/b/a; mixed (mainly parallel) beta-sheet of 8 strands, order | |
| | 34215786; strand 8 is antiparallel to the rest | |
| | 1. | Carbamate kinase-like (1) |
| | | topologically similar to the N-terminal domain of phosphoglycerate kinase |

| | |
|---|---|
| 1. | Carbamate kinase-like (2) |

| 74. | Class II aldolase (1) | |
|---|---|---|
| | 3 layers: a/b/a; mixed (mostly antiparallel) beta-sheet of 9 strands, order | |
| | 432159876; left-handed crossover between strands 4 and 5 | |
| | 1. | Class II aldolase (1) |

| | |
|---|---|
| 1. | Class II aldolase (1) |
| | metal (zinc)-ion dependent |

| 75. | Cytosolic phospholipase A2 catalytic domain (1) | |
|---|---|---|
| | 3 layers: a/b/a; mixed beta-sheet of 9 strands, order 654321789; strands 4, 6 | |
| | and 8 are antiparallel to the rest | |
| | 1. | Cytosolic phospholipase A2 catalytic domain (1) |

| | |
|---|---|
| 1. | Cytosolic phospholipase A2 catalytic domain (1) |

| 76. | Phosphatase/sulphatase (1) | |
|---|---|---|
| | 3 layers: a/b/a; mixed beta-sheet of 10 strands, order 564371892A, (A = 10) | |
| | strand 9 is antiparallel to the rest | |
| | 1. | Phosphatase/sulphatase (2) |

| | |
|---|---|
| 1. | Alkaline phosphatase (1) |
| 2. | Arylsulfatase (2) |

| 77. | Isocitrate & isopropylmalate dehydrogenases (1) | |
|---|---|---|
| | consists of two intertwined (sub)domains related by pseudodyad; duplication | |
| | 3 layers: a/b/a; single mixed beta-sheet of 10 strands, order 213A945867 | |
| | (A = 10); strands from 5 to 9 are antiparallel to the rest | |
| | 1. | Isocitrate & isopropylmalate dehydrogenases (1) |

| | |
|---|---|
| 1. | Isocitrate & isopropylmalate dehydrogenases (7) |

| 78. | ATC-like (2) | |
|---|---|---|
| | consists of two similar domains related bypseudodyad, duplication | |
| | core: 3 layers, a/b/a, parallel beta-sheet of 4 strands, order 2134 | |
| | 1. | Aspartate/ornithine carbamoyltransferase (1) |

| | |
|---|---|
| 1. | Aspartate/ornithine carbamoyltransferase (6) |

| | |
|---|---|
| 2. | Glutamate racemase (1) |

| | |
|---|---|
| 1. | Glutamate racemase (1) |
| | C-terminal extension is added to the N-terminal domain |

| 79. | Tryptophan synthase beta subunit-like PLP-dependent enzymes (1) | |
|---|---|---|
| | consists of two similar domains related by pseudodyad; duplication | |
| | core. 3 layers, a/b/a; parallel beta-sheet of 4 strands, order 3214 | |
| | 1. | Tryptophan synthase beta subunit-like PLP-dependent enzymes (1) |

| | |
|---|---|
| 1. | Tryptophan synthase beta subunit-like PLP-dependent enzymes (4) |

| 80. | SIS domain (1) | |
|---|---|---|
| | consists of two similar domains related by pseudodyad; duplication | |
| | 3 layers: a/b/a; parallel beta-sheet of 5 strands, order 21345 | |
| | 1. | SIS domain (2) |

| | |
|---|---|
| 1. | “Isomerase domain” of glucosamine 6-phosphate synthase |
| | (GLMS) (1) |
| 2. | Phosphoglucose isomerase, PGI (2) |
| | permutation of the superfamily fold |

| 81. | Formate dehydrogenase/DMSO reductase, domains 1-3 (1) | |
|---|---|---|
| | contains of two similar intertwined domains related bypseudodyad; | |
| | duplication | |
| | core: 3 layers: a/b/a; parallel beta-sheet of 5 strands, order 32451 | |
| | 1. | Formate dehydrogenase/DMSO reductase, domains 1-3 (1) |
| | | molybdopterine enzyme |

| | |
|---|---|
| 1. | Formate dehydrogenase/DMSO reductase, domains 1-3 (6) |
| | domain 1 (residues 1-55) binds Fe4S4 cluster in FDH but not |
| | DMSO reductase |

| 82. | Aldehyde reductase (dehydrogenase), ALDH (1) | |
|---|---|---|
| | consists of two similar domains with 3 layers (a/b/a) each; duplication | |
| | core: parallel beta-sheet of 5 strands, order 32145 | |
| | 1. | Aldehyde reductase (dehydrogenase), ALDH (1) |
| | | binds NAD differently from other NAD(P) -dependent oxidoreductases |

| | |
|---|---|
| 1. | Aldehyde reductase (dehydrogenase), ALDH (8) |

| 83. | Aconitase, first 3 domains (1) | |
|---|---|---|
| | consists of three similar domains with 3 layers (a/b/a) each; duplication | |
| | core: parallel beta-sheet of 5 strands, order 32145 | |
| | 1. | Aconitase, first 3 domains (1) |

| | |
|---|---|
| 1. | Aconitase, first 3 domains (2) |
| | contains Fe(′4,)-S(4) cluster |

| 84. | Phosphoglucomutase, first 3 domains (1) | |
|---|---|---|
| | consists of three similar domains with 3 layers (a/b/a) each; duplication | |
| | core: mixed beta-sheet of 4 strands, order 2134, strand 4 is antiparallel to the | |
| | rest | |
| | 1. | Phosphoglucomutase, first 3 domains (1) |

| | |
|---|---|
| 1. | Phosphoglucomutase, first 3 domains (1) |

| 85. | L-fucose isomerase, N-terminal and second domains (1) | |
|---|---|---|
| | consists of two domains of similar topology, 3 layers (a/b/a) each | |
| | Domain 1 (1-173) has parallel beta-sheet of 5 strands, order 21345 | |
| | Domain 2 (174-355) has parallel beta-sheet of 4 strands, order 2134 | |
| | 1. | L-fucose isomerase, N-terminal and second domains (1) |

| | |
|---|---|
| 1. | L-fucose isomerase, N-terminal and second domains (1) |

| 86. | Phosphoglycerate kinase (1) | |
|---|---|---|
| | consists of two non-similar domains, 3 layers (a/b/a) each | |
| | Domain 1 has parallel beta-sheet of 6 strands, order 342156 | |
| | Domain 2 has parallel beta-sheet of 6 strands, order 321456 | |
| | 1. | Phosphoglycerate kinase (1) |

| | |
|---|---|
| 1. | Phosphoglycerate kinase (4) |
| | Domain 2 binds ATP |

| 87. | UDP-Glycosyltransferase/glycogen phosphorylase (1) | |
|---|---|---|
| | consists of two non-similar domains with 3 layers (a/b/a) each | |
| | domain 1: parallel beta-sheet of 7 strands, order 3214567 | |
| | domain 2: parallel beta-sheet of 6 strands, order 321456 | |
| | 1. | UDP-Glycosyltransferase/glycogen phosphorylase (4) |

| | |
|---|---|
| 1. | beta-Glucosyltransferase (DNA-modifying) (1) |
| 2. | Peptidoglycan biosynthesys glycosyltransferase MurG (1) |
| 3. | UDP-N-acetylglucosamine 2-epimerase (1) |
| 4. | Oligosaccharide phosphorylase (4) |

| 88. | Glutaminase/Asparaginase (1) | |
|---|---|---|
| | consists of two non-similar alpha/beta domains, 3 layers (a/b/a) each | |
| | Domain 1 has mixed beta-sheet of 6 strands, order 213456, strand 6 is | |
| | antiparallel to the rest; left-handed crossover connection between strands 4 and 5 | |
| | Domain 2 has parallel beta-sheet of 4 strands, order 1234 | |
| | 1. | Glutaminase/Asparaginase (1) |

| | |
|---|---|
| 1. | Glutaminase/Asparaginase (5) |

| 89. | Phosphofructokinase (1) | |
|---|---|---|
| | consists of two non-similar domains, 3 layers (a/b/a) each | |
| | Domain 1 has mixed sheet of 7 strands, order 3214567; strands 3 & 7 are | |
| | antiparallel to the rest | |
| | Domain 2 has parallel sheet of 4 strands, order 2314 | |
| | 1. | Phosphofructokinase (1) |

| | |
|---|---|
| 1. | Phosphofructokinase (2) |
| | Domain 1 binds ATP |

| 90. | Cobalt precorrin-4 methyltransferase CbiF (1) | |
|---|---|---|
| | consists of two non-similar domains | |
| | Domain 1 has antiparallel sheet of 5 strands, order 32415 | |
| | Domain 2 has mixed sheet of 5 strands, order 12534; strands 4 & 5 are | |
| | antiparallel to the rest | |
| | 1. | Cobalt precorrin-4 methyltransferase CbiF (1) |

| | |
|---|---|
| 1. | Cobalt precorrin-4 methyltransferase CbiF (1) |

| 91. | Phosphoenolpyruvate carboxykinase (ATP-oxaloacetate carboxy-liase) (1) | |
|---|---|---|
| | consists of two alpha/beta domains | |
| | duplication: the domains share an unusual fold of 2 helices and 6-stranded | |
| | mixed sheet; beta(2)-alpha-beta(4)-alpha; order 312465, strands 1 and 5 are | |
| | antiparallel to the rest | |
| | 1. | Phosphoenolpyruvate carboxykinase (AIP-oxaloacetate carboxy-liase) (1) |
| | | domain 2 contains the P-loop ATP-binding motif |
| | 1. | Phosphoenolpyruvate carboxykinase (ATP-oxaloacetate carboxy-liase) (1) |
| 92. | Chelatase-like (2) | |
| | duplication: tandem repeat of two domains; 3 layers (a/b/a); parallel | |
| | beta-sheet of 4 strands, order 2134 | |
| | 1. | Chelatase (2) |
| | | interdomain linker is short; swapping of C-terminal helices between |
| | | the two domains |

| | |
|---|---|
| 1. | Ferrochelatase (1) |
| 2. | Cobalt chelatase CbiK (1) |

| | |
|---|---|
| 2. | “Helical backbone” metal receptor (3) |
| | contains a long alpha helical insertion in the interdomain linker |

| | |
|---|---|
| 1. | Periplasmic ferric siderophore binding protein FhuD (1) |
| 2. | TroA-like (2) |
| 3. | Nitrogenase iron-molybdenum protein (3) |
| | contains three domains of this fold; “Helical backbone” holds |
| | domains 2 and 3 |

| 93. | Periplasmic binding protein-like I (1) | |
|---|---|---|
| | consists of two similar intertwined domain with 3 layers (a/b/a) each: | |
| | duplication | |
| | parallel beta-sheet of 6 strands, order 213456 | |
| | 1. | Periplasmic binding protein-like I (1) |
| | | Similar in architecture to the superfamily II but partly differs in |
| | | topology |

| | |
|---|---|
| 1. | L-arabinose binding protein-like (13) |

| 94. | Periplasmic binding protein-like II (1) | |
|---|---|---|
| | consists of two similar intertwined domain with 3 layers (a/b/a) each: | |
| | duplication | |
| | mixed beta-sheet of 5 strands, order 21354; strand 5 is antiparallel to the rest | |
| | 1. | Periplasmic binding protein-like II (2) |
| | | Similar in architecture to the superfamily I but partly differs in |
| | | topology |

| | |
|---|---|
| 1. | Phosphate binding protein-like (20) |
| 2. | Transferrin (8) |
| | further duplication; composed of two two-domain lobes |

| 95. | Thiolase-like (1) | |
|---|---|---|
| | consists of two similar domains related by pseudodyad; duplication | |
| | 3 layers: a/b/a; mixed beta-sheet of 5 strands, order 32451; strands 1 & 5 are | |
| | antiparallel to the rest | |
| | 1. | Thiolase-like (2) |

| | |
|---|---|
| 1. | Thiolase-related (6) |

| | 2. | Chalcone synthase (2) |
|---|---|---|
| 96. | Fe-only hydrogenase (1) | |
| | consist of two intertwined domains; contains partial duplication | |
| | 1. | Fe-only hydrogenase (1) |

| | |
|---|---|
| 1. | Fe-only hydrogenase (2) |

| 97. | Cytidine deaminase (1) | |
|---|---|---|
| | consists of two very similar domains with 3 layers (a/b/a) each; duplication | |
| | mixed beta-sheet of 4 strands, order 2134; strand 2 is antiparallel to the rest | |
| | 1. | Cytidine deaminase (1) |

| | |
|---|---|
| 1. | Cytidine deaminase (1) |

TABLE 2

| | |
|---|---|
| Cmpd A | 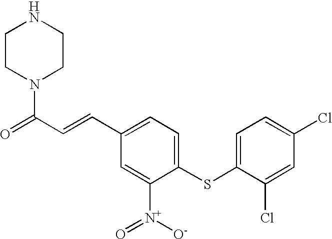 |
| Cmpd B | 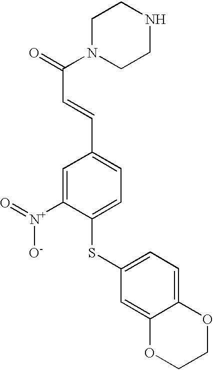 |
| Cmpd C | 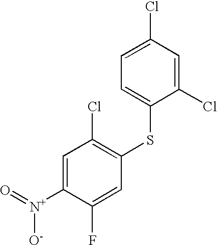 |
| Cmpd D | 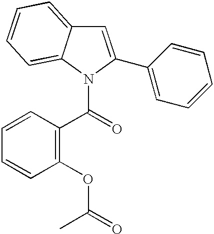 |
| Cmpd E | 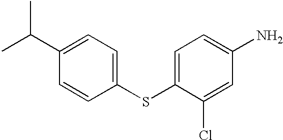 |
| Cmpd F | 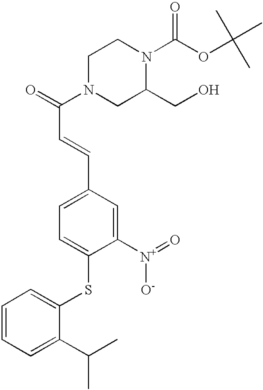 |
| Cmpd G | 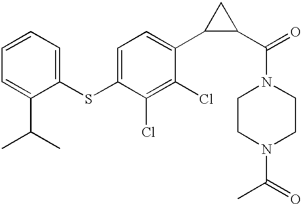 |
| Cmpd H | 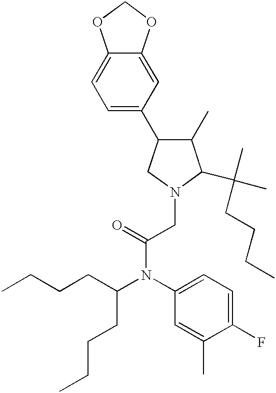 |
| Cmpd I | 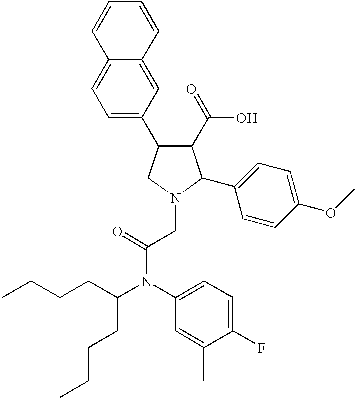 |
| Cmpd J | 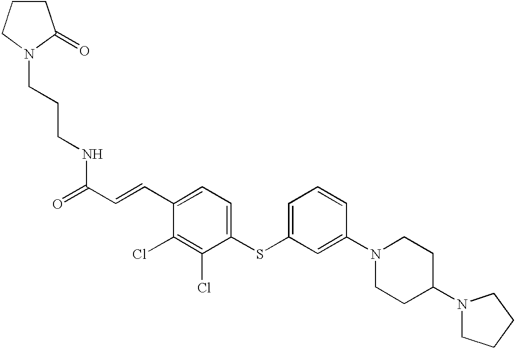 |
| Cmpd K | 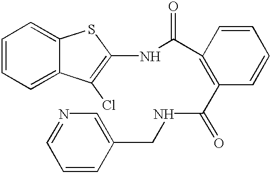 |
| Cmpd L | 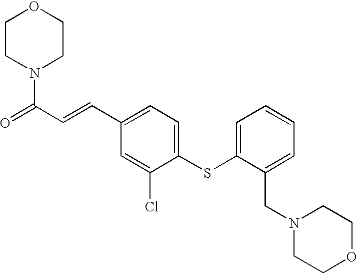 |
| Cmpd M | 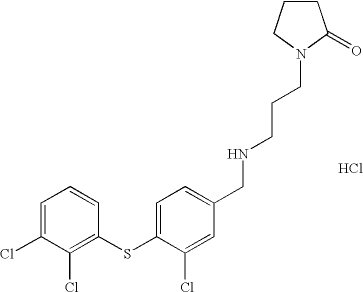 |
| Cmpd N | 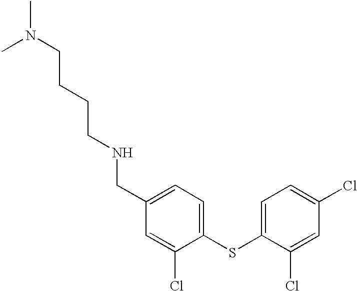 |
| Cmpd O | 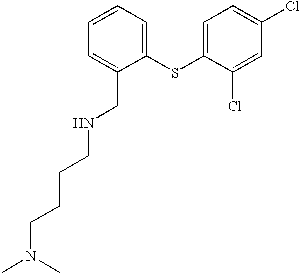 |
| Cmpd P | 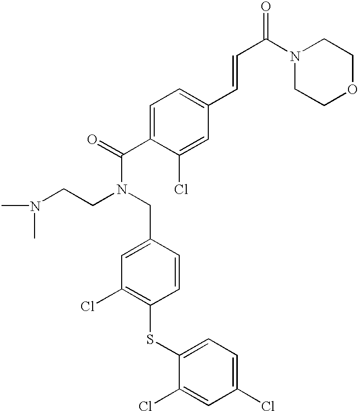 |
| Cmpd Q | 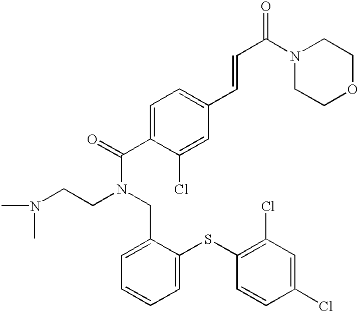 |
| Cmpd R | 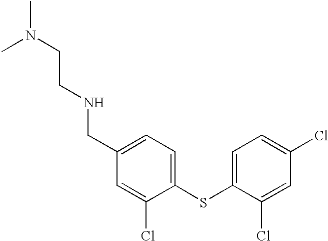 |
| Cmpd S | 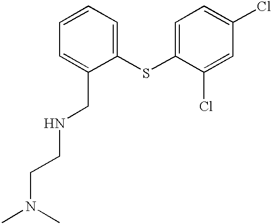 |
| Cmpd T | 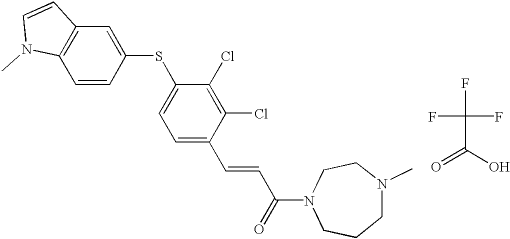 |
| Cmpd U | 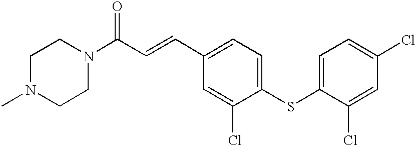 |
| Cmpd V | 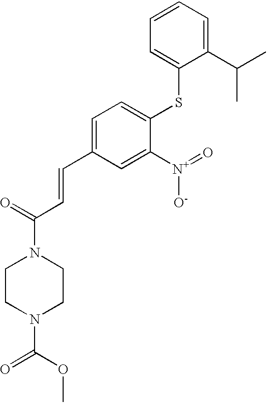 |
| Cmpd W | 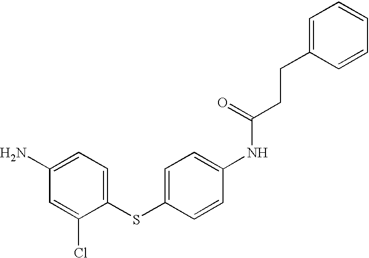 |
| Cmpd X | 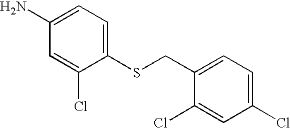 |
| Cmpd Y | 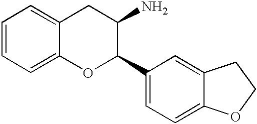 |
| Cmpd Z | 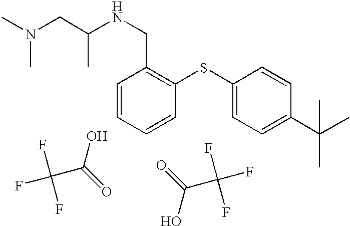 |
| Cmpd AA | 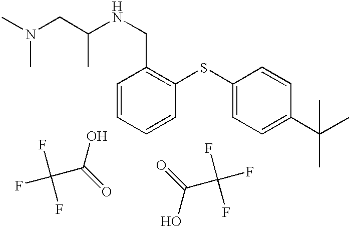 |
| Cmpd AB | 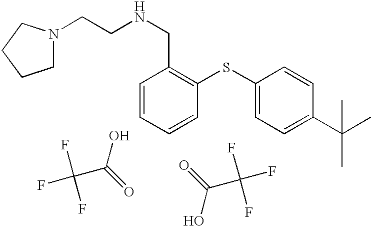 |
| Cmpd AC | 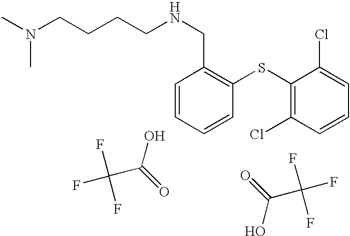 |
| Cmpd AD | 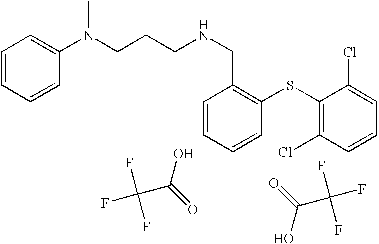 |
| Cmpd AE | 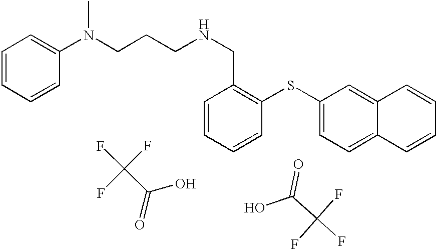 |
| Cmpd AF | 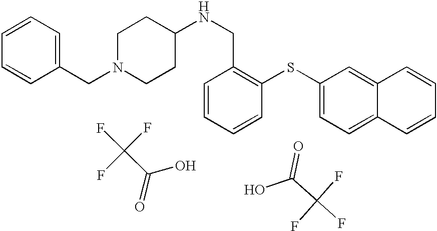 |
| Cmpd AG | 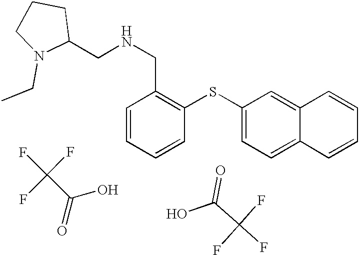 |
| Cmpd AH | 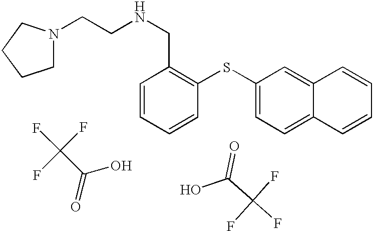 |
| Cmpd AI | 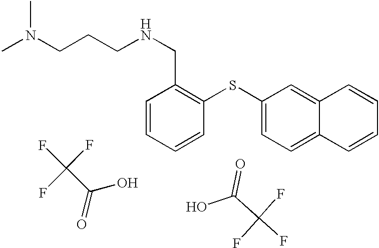 |
| Cmpd AJ | 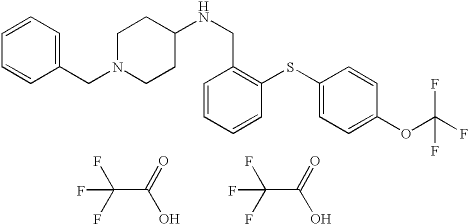 |
| Cmpd AK | 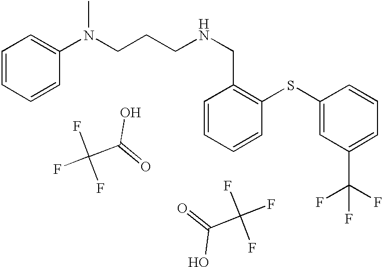 |
| Cmpd AL | 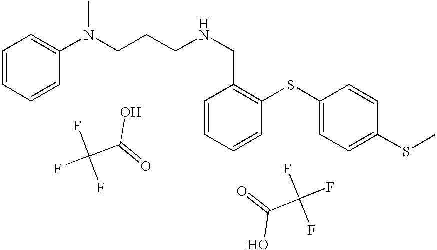 |
| Cmpd AM | 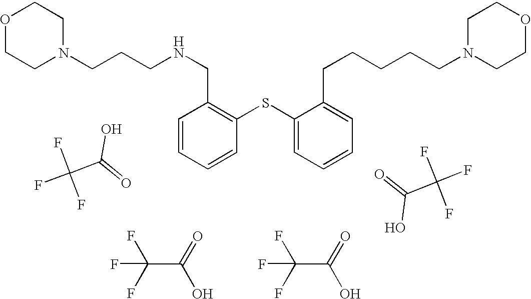 |
| Cmpd AN | 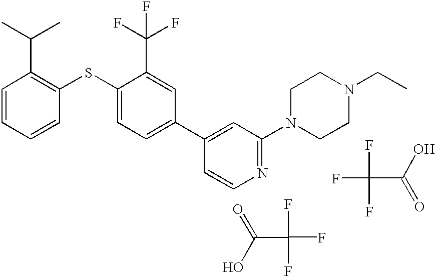 |
| Cmpd AO | 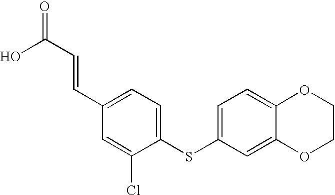 |
| Cmpd AP | 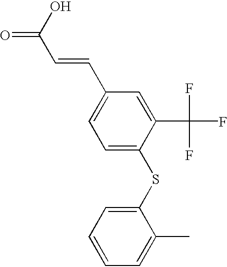 |
| Cmpd AQ | 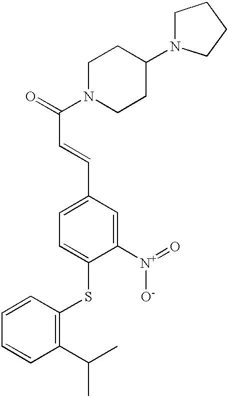 |
| Cmpd AR | 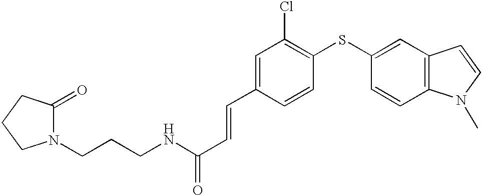 |
| Cmpd AS | 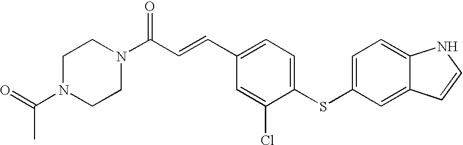 |
| Cmpd AT | 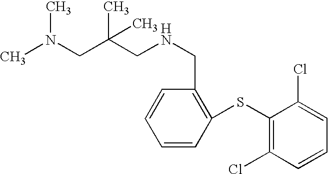 |
| Cmpd AU | 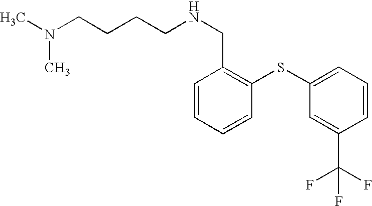 |
| Cmpd AV | 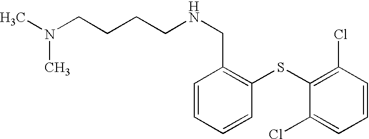 |
| Cmpd AW | 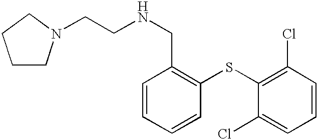 |
| Cmpd AX | 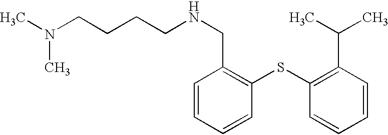 |
| Cmpd AY | 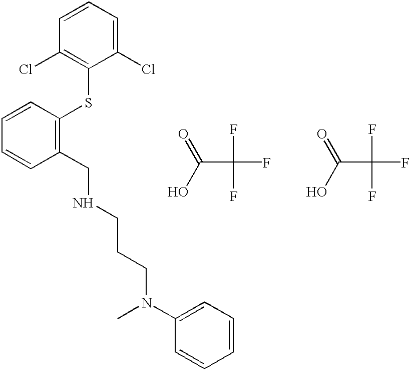 |
| Cmpd AZ | 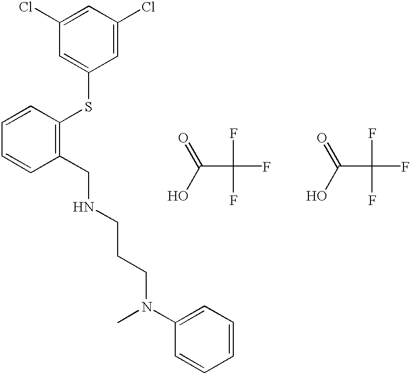 |
| Cmpd AAA | 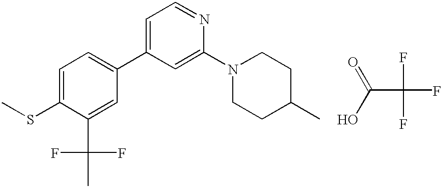 |
| Cmpd AAB | 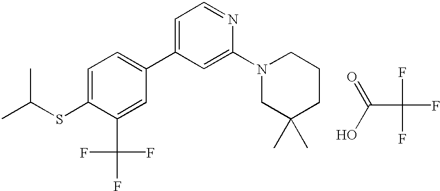 |
| Cmpd AAC | 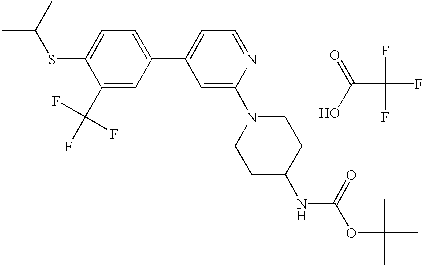 |
| Cmpd AAD | 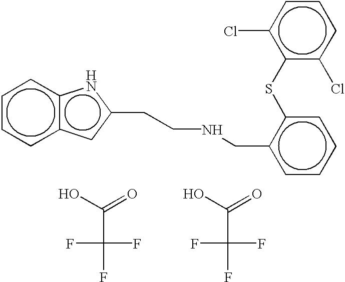 |
| Cmpd AAE | 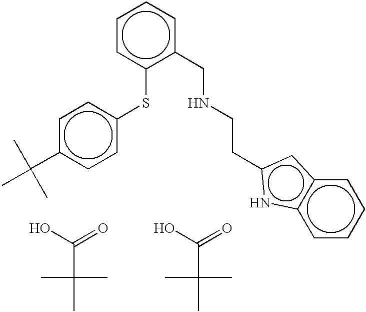 |
| Cmpd AAF | 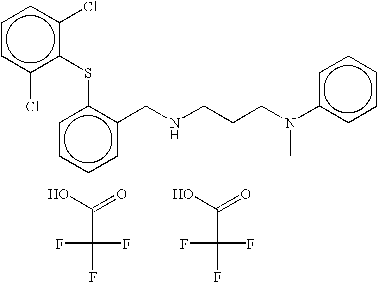 |
| Cmpd AAG | 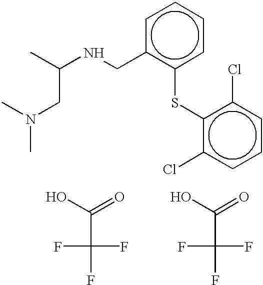 |

The present invention is illustrated by the following examples.

### EXAMPLE 1

### Identification of Alpha/Beta Proteins and Allosteric Regulatory Sites

The present invention also provides methods of identifying a molecule which is not LFA-1 or an I domain containing fragment thereof, said molecule comprising an α/β domain structure, said α/β structure comprising an allosteric regulatory site. When said molecule is contacted with an allosteric effector molecule, allosteric regulatory sites such as, for example, I domain allostenc sites, interact with said allosteric effector molecule to promote a conformation in a ligand binding domain of said α/β structure that modulates binding between the first molecule and a binding partner molecule thereof.

Allosteric regulatory sites can be identified, for example, by comparing candidate proteins to proteins having known allosteric regulatory sites. For example, α/β proteins having allosteric regulatory sites may be identified by using search tools, such as a NCBI vector alignment search tool (or “VAST” search), which are able to identify proteins similar to a predetermined three dimensional structure [Gibrat et al., Curr. Opin. Struct. Biol. 6:377-385 (1996)], incorporated by reference herein in its entirety; and, Madej et al., Proteins 23:356-369 (1995), incorporated by reference herein in its entirety]. With respect to these methods, LFA-1 can be used as a comparison or query protein because LFA-1 is known to include an I domain allosteric site. Similarly, other α/β proteins known to comprise an allosteric site can be used as a reference to identify other α/β proteins comprising an I domain allosteric site. In one embodiment, proteins with a VAST score of 7 or greater or a P value of 0.005 or less may be defined as being sufficiently related to the comparison protein to warrant further investigation.

Allosteric regulatory sites may also be identified by using an algorithm that predicts conformational ambivalence [Young et al., Protein Science 8:1752-1764 (1999), incorporated by reference herein in its entirety; and, Kirshenbaum et al. Protein Science 8(9):1806-1815 (1999), incorporated by reference herein in its entirety]. This algorithm, referred to as the Ambivalent Structure Predictor (“ASP”), predicts regions of three-dimensional conformational rearrangement from amino acid sequence information. The algorithm uses scaled probabilities from a secondary structural prediction algorithm, Profile Network Prediction Heidelberg (“PHD”) [Rost, Meth. Enzymol. 266:525-539 (1996), incorporated by reference herein in its entirety], to identify structurally ambivalent sequence elements. Residues possessing a z score below −1.75 standard deviations of the mean residue ambivalence score in α/β domains are understood as being consistent with an allosteric regulatory site of the type useful according to the present invention.

For example, Table 3 shows that the integrin α/β domains and their close relatives possess a high VAST core of approximately 10 or greater and a P value of approximately 0.0009 or less relative to two representatives LFA-1 and Mac-1. Further, Table 3 indicates that the position of structurally ambivalent sequence elements (SASE) is consistent with the known or predicted c-terminal rigid body motion for these domains. Accordingly, these and other closely related domains of this type are predicted to possess a typical IDAS. Moreover, as demonstrated by the calculations presented in Table 3, some Ras superfamily members such as RhoA and enzymes such as ENR are also predicted to possess a typical IDAS.

Additionally, some non-integrin α/β domains that are more distantly related, as demonstrated by VAST analysis, possess a SASE at a site that appears to be distinct from the typical integrin IDAS. These α/β domains may possess an IDAS-like site also capable of being modulated with a small molecule such as a diaryl compound.

Many α/β domains share less than 35% amino acid identity. Therefore, a web-based simple modular architecture research tool, SMART, [see Schultz et al., Nuc. Acids Res., 28:231-234 (2000), incorporated by reference herein in its entirety; Copley et al., Curr. Opin. Struct. Biol. 9:408-415 (1999), incorporated by reference herein in its entirety; Ponting et al., Nuc. Acids Res. 27:229-232 (1999), incorporated by reference herein in its entirety; and, Schultz et al., PNAS USA 95:5857-5864 (1998), incorporated by reference herein in its entirety] that compares query sequences with its database of domain sequences has been used to identify additional divergent family members. SMART utilizes multiple sequence alignments of representative family members. These alignments are optimized manually, and following the generation of a hidden Markov model, can be used to search sequence databases. Significantly similar sequences are added to the alignment, thereby refining the model which is used for subsequent searches. Accordingly, the SMART database may be used as a source of identifying additional α/β domains of interest to analyze for the presence of an allosteric regulatory site.

TABLE 3

| | | | | | ASP SASE* |
|---|---|---|---|---|---|
| | | | | | position |
| | VAST Structure Neighbor | | | | (Residues |
| | LFA-1 | | Mac-1 | | from C- |
| αβ domain | Score | P value | Score | P value | Termini) |
| α_{L}(LFA-1, 1Z00) | — | — | 14.7 | 10e−11.7 | 27 |
| α_{M}(Mac-1, 1IDN) | 13.2 | 10e−4.8 | — | — | 28 |
| α₁(1QC5) | 13.8 | 10e−11.6 | 17.6 | 10e−15.9 | 23 |
| α₂(IDZ1A) | 12.5 | 10e−9.0 | 17.2 | 10e−15.3 | 16 |
| ENR(IDFIA) | 12.2 | 0.0009 | 10.8 | 0.0001 | 11 |
| G_{α1}(1GFI) | | | 12.4 | 0.0016 | 70‡ |
| Rac1(1MH1) | | | 11.6 | 0.029 | † |
| RhoA(1DPFA) | | | 12.1 | 0.0045 | 23 |
| cdc42(1AM4D) | | | 11.6 | 0.253 | 20 |
| H-Ras(1Q21) | 10.4 | 0.0406 | 12.2 | 0.0027 | † |
| Sir2(1ICIA) | | | 8.0 | 0.0088 | 56‡ |
| ftsZ(1FSZ) | 11.7 | 0.0277 | 14.4 | 0.0048 | 92‡ |
| HPPK(1DY3A) | | | | | 37‡ |
| Era (1EGA) | 9.8 | 0.0474 | 13.3 | 0.001 | 81‡ |

*SASE: Structurally ambivalent sequence element.
†C-Terminal SASE not detected by ASP default settings.
‡Second site of SASE may represent IDAS-like site.

### EXAMPLE 2

### CD11b I Domain Mutants

A. Generation of Mutations in the CD11b I Domain

In view of previous results [Huth, et al., Proc. Natl. Acad. Sci. (USA) 97:5231-5236 (2000)] using CD11a variants with mutations in the I domain, mutations were introduced in CD11b in an attempt to identify CD11b variants with increased affinity for binding partners ICAM-1 and iC3b.

Six mutations were generated using a QuikChange Site-Directed Mutagenesis Kit (Stratagene). These mutants included single changes of Asp¹⁵⁶ (D156A), Val²⁵⁴ (V254A), Gln³²⁷ (Q327A), Ile³³² (I332A), Phe³³³ (F333A) and Glu³³⁶ (E336A) to Ala. Briefly, two mutagenic oligonucleotides (one to the sense strand and one to the antisense strand) were synthesized which were used in PCR with full-length CD11b as template. The PCR conditions for mutants D156A, V254A, Q327A, and I332A included 1 cycle at 95° C. for 30 seconds followed by 16 cycles of 95° C. for 30 seconds, 50° C. for 1 minute and 60° C. for 18 minutes. PCR conditions for mutants F333A and E336A were the same except that the final elongation step was carried out at 68° C. for 20 minutes in the 16 cycles. After the PCR was complete, the methylated, non-mutated template DNA was digested with DpnI at 37° C. for 1 hour and the mutagenized CD11b DNA was used to transform Supercompetent XL1 Blue Cells (Stratagene) according to the manufacturer's suggested protocol. Carbomycin resistant colonies were picked and grown in liquid culture, after which plasmid DNA was isolated and the insert was sequenced. From clones having full-length mutants, a 1.3 kb SacI/EcoRV fragment containing the 5′ portion of the gene was subcloned back into the parental vector. The inserts from these subclones were sequenced to verify the integrity of the junctions and the presence of the mutation.

| | | |
|---|---|---|
| D156A (sense) | | SEQ ID NO:1 |
| | CATTGCCTTCTTGATTGCGGGCTCTGGTAGCATC | |
| V254A (sense) | | SEQ ID NO:2 |
| | GCCTTTAAGATCCTAGCGGTCATCACGGATGGAG | |
| Q327A (sense) | | SEQ ID NO:3 |
| | GAAGACCATTCAGAACGCGCTTCGGGAGAAGATC | |
| 1332A (sense) | | SEQ ID NO:4 |
| | CAGCTTCGGGAGAAGGCGTTTGCGATCGAGGG | |
| F333A (sense) | | SEQ ID NO:5 |
| | CTTCGGGAGAAGATCGCGGCGATCGAGGGTAC | |
| E336A (sense) | | SEQ ID NO:6 |
| | GAAGATCTTTGCGATCGCGGGTACTCAGACAGG | |

B. COS-7 Transfections

COS cells were co-transfected with CD18/pDC1 and either wild-type CD11b or a mutant form of CD11b. Transfections were performed essentially as previously described [Huth, et al., Proc. Natl. Acad. Sci (USA) 97:5231-5236 (2000)].

C. FACS Analysis

FACS analysis was carried out as previously described [Huth, et al., Proc. Natl. Acad. Sci. (USA) 97:5231-5236 (2000)] except that the anti-CD11b monoclonal antibody TMG6-5 [Diamond, et al., J. Cell Biology 120:1031-1043 (1993)] was used to confirm CD11b expression.

D. Adhesion Assay with COS Transfected Cells and Immobilized ICAM-1 or iC3b

Adhesion assays were performed in 96-well Easy Wash plates (Corning Glass, Corning, N.Y.) using a modified procedure [Sadhu, et al., Cell Adhes. Commun. 2:429-440 (1994)]. Each well was coated overnight at 4° C. with 50 μl of glycophorin (Calbiochem) (10 μg/ml), ICAM-1/Fc (5 μg/ml), iC3b (3 μg/ml) or with anti-CD18 monoclonal antibody (TS1/18, 5 μg/ml) and anti-CD11b monoclonal antibody (44AACB [ATCC], 5 μg/ml) in 50 mM bicarbonate buffer (pH 9.6), or buffer alone. Plates were washed twice with 200 μl/well D-PBS and blocked with 1% HSA (100 μl/well) in D-PBS for 1 hr at room temperature. Wells were rinsed once with 100 μl of adhesion buffer (containing RPMI and 5.0% inactivated FBS) and 100 μl adhesion buffer was added to each well. Another 100 μl of adhesion buffer, with or without control antibody (IgG(5a)7.2, 60 μg/ml), blocking antibody (44AACB, 60 μg/ml) or activating antibody 240Q [Huth, et al., Proc. Natl. Acad. Sci. (USA) 97:5231-5236 (2000)] at 60 μg/ml was added to each well, after which COS-7 transfectants (100 μl of 0.75×10⁶ cells/ml) in adhesion buffer were added to each well. The plates were incubated at 37° C. for 30 minutes for ICAM-1 binding or 15 minutes for iC3b binding. Adherent cells were fixed by the addition of 50 μl/well 14% glutaraldehyde in D-PBS and incubation continued at room temperature for 1.5 hr. The plates were washed with dH₂O, stained with 100 μl/well 0.5% crystal violet in 10% ethanol for 5 minutes at room temperature, and washed in several changes of dH₂O. After washing, 70% ethanol was added and adherent cells were quantitated by determining absorbence at 570 nm and 410 nm using a SPECTRmax 250 microplate spectrophotometer system (Molecular Devices, Sunnyvale, Calif.). Percentage of cell binding was determined using the formula below.

$$\% of cell binding = \frac{\begin{matrix}A570 - A410 ( binding to \\ ICAM \text{-1} or iC3b )\end{matrix}}{\begin{matrix}A570 - A410 ( binding to CD18 + \\ CD11b monoclonal antibodies )\end{matrix}} \times 100$$

Results indicated that wild type CD11b binding to ICAM-1 and iC3b was 3.1% and 26.4%, respectively. Mutants V254A, Q327A, and I332A each demonstrated significantly higher binding to ICAM-1 (114.7%, 105.1%, and 123.1% of wildtype levels, respectively) and iC3b (147.1%, 140.5%, and 205.2%, respectively), while mutants F332A and E336A showed significantly lower binding to both ICAM-1 (1.1% and 0.7%, respectively) and iC3b (4.9% and 4.3%, respectively). Mutants which demonstrate higher levels of ICAM-1 binding are therefore useful for identifying compounds that inhibit CD18/CD11b (Mac-1) binding to ICAM-1 in providing a higher signal-to-noise ratio as a result of the increased level of ICAM-1 binding.

### EXAMPLE 3

### Identification of CD11b Agonists

Previous work has demonstrated that various diaryl compounds can inhibit LFA-1 binding to ICAM-1. In view of this observation and the results in Example 1 above, experiments were designed to determine if diaryl compounds can affect CD11b binding to natural binding partners, presumably through interaction with an allosteric regulatory region of CD11b.

A. Adhesion Assay of HL60 Expressing α_{M} to Immobilized ICAM-1

In order to assess the ability of the test compounds to modulate CD11b (α_{M}) binding, adhesion assays were performed using HL60 cells and immobilized ICAM-1.

Assays were performed in the presence of blocking anti-CD18 monoclonal antibody (TS1/22, 10 μg/ml) with 100 μl of HL60 cells (1×10⁶ cells/ml) in adhesion buffer were performed in 96-well Easy Wash plates (Corning Glass, Corning, N.Y.) using the procedure described above except that each well was coated overnight at 4° C. with (i) 50 μl ICAM-1/Fc (5 μg/ml), (ii) anti-CD18 monoclonal antibody (22F12C, 5 μg/ml) and anti-alpha 4 monoclonal (A4.1, 5 μg/ml) in 50 mM bicarbonate buffer (pH 9.6), or (iii) buffer alone. Percentage of cell binding was determined using the formula below.

$$\% Binding = \frac{A570 - A410 ( binding to ICAM \text{-1} )}{\begin{matrix}A570 - A410 ( binding to CD18 + \\ CD11a mAb )\end{matrix}} \times 100$$

Data was then normalized using the formula:

$$\% of DMSO binding = \frac{\% of cell binding , inhibitors}{\% of cell binding , DMSO} \times 100$$

Approximately 30 compounds were identified for further study. IC50 values were determined in the HL-60 assay described above or in a neutrophil binding assays with fibrinogen described below (Example 15).

B. Adhesion Assay of JY/CD11b Cells to Immobilized iC3b

Briefly, each well of a 96-well plate was coated overnight at 4° C. with 50 μl glycophorin (10 μg/ml), iC3b (5 μg/ml) or with anti-CD18 monoclonal antibody (22F12C, 5 μg/ml) and anti-CD11b monoclonal antibody (44AACB, 5 82 g/ml) in bicarbonate buffer (pH 9.6). Plates were blocked with human serum albumin in D-PBS for one hr at room temperature. JY cells transfected with CD11b (JY/CD11b cells) (100 μl at 1×10⁶ cells/ml) in adhesion buffer were added to each well and incubation was carried out at 37° C. for 30 min. Plates were fixed and analyzed as described above in Example 1. Percentage of cells binding was determined using the equation below.

$$\% Binding = \frac{( A570 - A410 ( binding to iC3b )}{\begin{matrix}A570 - A410 ( binding to CD18 + \\ CD11b mAbs )\end{matrix}} \times 100$$

Data was normalized using the formula:

$$\% of DMSO binding = \frac{\% of cell binding , inhibitors}{\% of cell binding , DMSO} \times 100$$

IC50 values were determined for 45 compounds that demonstrated inhibition in the screen and six of these compounds showed IC50 of less than 10 μM. Twelve of the 45 compounds were subsequently used in binding assays using neutrophil adhesion to fibrinogen (described in Example 15).

This screen also identified 17 compounds with the ability to stimulate binding to iC3b. Re-titration of these 17 compounds revealed that Cmpd H, Cmpd I, and Cmpd C were capable of dose-dependent stimulation of CD11b/CD18 binding to iC3b at a level two times that observed with control DMSO treatment.

### EXAMPLE 4

### Screening for Inhibitors of Complement Protein C2 and Factor B

Complement proteins C2 and Factor B have been shown to include A domain regions which are believed to regulate serine protease activity of the proteins and their respective convertases. The A domains in these proteins are also believed to serve as ligand binding sites and to include one or more regulatory domains. C2 binds complement protein C4b to form the C3 convertase and part of the C5 convertase in the classical complement pathway, and Factor B binds C3b to form the alternative complement pathway C3 convertase and part of the C5 convertase. Identification of modulators for C2 or Factor B binding would presumably provide a mechanism by which C3 and/or C5 convertase activity can be controlled.

A screen for inhibitors of the classical pathway complement protein C2 and alternative pathway complement protein Factor B includes primary screening using modifications of standard hemolytic CH50 and AH50 assays in a microtiter plate format as described below. [See also Current Protocols in Immunology, Chapter 13, Unit 13.1, John Wiley & Sons, Inc., ( 2000).] The CH50 assay is dependent on the activity of the classical pathway and C2, whereas the AH50 assay is dependent on the activity of the alternative pathway and Factor B.

The CH50 assay consists of analysis of complement-dependent lysis of sheep red blood cells (RBCs) which have been opsonized with anti-sheep RBC serum and is dependent on both Mg⁺⁺ and Ca⁺⁺. The CH50 is the concentration of human serum necessary to cause the lysis of 50% of the opsonized sheep RBC within 1 hour at 37° C. The primary screen for C2 inhibitors includes use of a constant serum concentration at the CH50 level, and the assay is conducted in the presence and absence of 10 μM of test compounds. Compounds that inhibit this primary assay are titrated and retested for specificity in a secondary hemolytic assay in which each individual purified complement protein is added sequentially in the presence or absence of the test compound to determine which component is being inhibited.

The AH50 assay consists of analysis of the direct complement-dependent lysis of rabbit red blood cells and is dependent on Mg⁺⁺ but not Ca⁺⁺, and therefore is performed in the presence of EGTA. Similar to the CH50, the AH50 is the concentration of human serum necessary to cause the lysis of 50% of the rabbit RBC within 1 hour at 37° C. The primary screen for Factor B inhibitors includes use of a serum concentration at the AH50 level, and the assay is conducted in the presence and absence of 10 μM of test compounds. Compounds that inhibit this primary assay are titrated and retested for specificity in a secondary hemolytic assay in which each individual purified complement protein is added sequentially in the presence or absence of the compound to determine which component is being inhibited.

Sheep whole blood in Alsevers solution and anti-sheep hemolysin were obtained from Colorado Serum Co. (Denver, Colo.). Erythrocyte-antibody complexes (EA) were produced using an optimal concentration of anti-sheep hemolysin, determined by titration to be a 1:800 dilution. Normal human serum (NHS) was generated by collecting fresh serum from 10 random healthy human donors, pooling it, aliquotting the pooled serum, flash freezing it in liquid nitrogen, and storing it at −70° C. A fresh aliquot was thawed immediately prior to each use.

A standard assay was established in Costar 96-well round-bottom or V-bottom microtiter plates. All samples were analyzed in duplicate and averaged. First, the NHS was titrated to determine the midpoint of its linear activity in lysing the EA (the CH50 dilution). Senral two-fold dilutions of freshly thawed NHS in gelatin-veronal buffer with Mg⁺⁺ and Ca⁺⁺ (GVB⁺⁺ containing 0.142 M NaCl, 4.9 mM sodium 5, 5′-diethylbarbituric acid and 1.0 g/l gelatin, the pH adjusted to 7.35 with HCl, followed by addition of CaCl₂ and MgCl₂ to final concentrations of 60 μM and 400 μM, respectively), or dH₂O (used to determine total lysis) were placed in duplicate wells (80 μl/well) and warmed to 37° C. for 5 minutes. EA which had been washed twice with GVB⁺⁺ and resuspended at 2×10⁸ complexes/ml were added (80 μl/well) and the plate was incubated at 37° C. for 60 minutes. Eighty μl/well of 0.15 M NaCl was added and the plate was centrifuged at 2500 rpm for 3 minutes. One hundred μl/well of supernatant was transferred from the assay plate to an Immulon4 96-well flat-bottom ELISA plate and the absorbance at 420 nm was determined. Background readings of absorbance in the wells containing no NHS were subtracted from the reading for all wells containing NHS and the resulting specific absorbance was expressed as a percentage of that obtained from wells containing dH₂O (% Total Lysis).

The dilution of NHS necessary to give 50% Total Lysis in 60 minutes at 37° C. (the CH50) was determined to be 1:150. This dilution constituted the midpoint of the linear range of the NHS lytic activity and was used to screen the library of test compounds for inhibitors of the complement pathway. The test compounds were first diluted in GVB⁺⁺/5% DMSO to 40 μLM and aliquotted at 40 μl/well in duplicate into Costar 96-well round-bottom or V-bottom plates. Control wells containing GVB⁺⁺ (background), dH₂O (total lysis), DMSO alone, anti-C2 polyclonal antisera (40 μg/ml; Calbiochem), normal goat IgG (40 μg/ml; Sigma), and EGTA (4 mM) were also included. Plates were incubated at 37° C. for five minutes. Forty μl/well of NHS diluted to 1:75 in GVB⁺⁺ was added (this created a 1:150 final dilution with compound), except in background or total lysis wells which received GVB⁺⁺ or dH₂O, respectively. Plates were incubated at 37° C. for 10 minutes. EA were washed twice, resuspended at 2×10⁸/ml in GVB⁺⁺, and added to each plate at 80 μl/well. The plates were incubated at 37° C. for 60-70 minutes, after which 80 μl/well of 0.15 M NaCl was added and the plates were centrifuged at 2500 rpm for 3 minutes. One hundred μl of supernatant from each well was transferred from the assay plates to separate wells on Immulon4 96-well flat-bottom ELISA plates and the absorbance at 420 nm was analyzed. Background readings of absorbance in the wells containing no NHS were subtracted from the absorbance for each well and the resulting specific absorbance was expressed as a percentage of that obtained from wells containing DMSO alone (% DMSO lysis). All compounds which inhibited DMSO lysis by greater than 35% were re-tested and titrated in the same assay. Thirty nine compounds were identified with IC50 values of less than or equal to 20 μM. The two most potent compounds had IC50 values of less than 5 μM, and were shown to be selective for complement inhibition since they did not significantly inhibit (i) LFA-1 mediated adhesion to ICAM-1, (ii) Mac-1 mediated adhesion to ICAM-1, (iii) α₂β₁ mediated adhesion to collagen, (iv) α₄β₇ mediated adhesion to MAdCAM-1, or (v) vWf binding to gp1b in standard cell-based adhesion assays at concentrations greater than or equal to 20 μM.

Approximately 30% of the activity of serum in the classical complement pathway (CCP) screen is due to amplification by the alternative complement pathway (ACP) Factor B containing C3 and C5 convertases. Therefore, this assay has the potential to isolate inhibitors of either the classical complement pathway convertases, the lectin complement pathway (LCP) (in which C3 is an intermediate component as well), and the alternative complement pathway. It is also possible that given the high degree of primary structural homology between C2 and Factor B, compounds may be isolated which inhibit both convertases in all three pathways.

Given the nature of the original screen, inhibition could have occurred at any stage of the complement pathway. In order to determine at which stage of complement activation the test compounds inhibited activity, purified complement proteins were obtained (Advanced Research Technologies, San Diego, Calif.) and complement activation was reconstituted in a stepwise manner. At each step, the lead compound or DMSO alone was added and the terminal hemolytic activity was measured as above. Initially, the lead compound was tested for its ability to inhibit at any of four different stages of complement activation: 1) C1 binding to aggregated antibody on the surface of the EA; 2) C4 binding to and cleavage by C1; 3) C2 binding to C4b, activation of C2 by C1-mediated cleavage and C4bC2a-mediated cleavage of C3 (i.e., formation and activity of the C3 convertase); and 4) formation and activity of the C5 convertase and subsequent deposition of complement proteins C6 through C9, which form the membrane attack complex (MAC) resulting in cell lysis.

In the assay, 1×10⁷ EA/well were analyzed in duplicate wells of Costar 96-well round-bottom plates. For testing stage 1 (as indicated above), cells were resuspended in GVB⁺⁺ containing 7.5 μg/ml C1 protein and incubated for 15 minutes at 30° C. For testing stage 2, cells were resuspended in GVB⁺⁺ containing 7.5 μg/ml C4 protein and incubated for 15 minutes at 30° C. For testing stage 3, cells were resuspended in GVB⁺⁺ containing 0.4 μg/ml C2 protein and 25 μg/ml C3 protein and incubated for 30 minutes at 30° C. For testing stage 4, cells were resuspended in GVB⁺⁺ containing 4 mM EGTA and a 1:50 dilution of NHS and incubated for 60 minutes at 37° C. For each stage, a titration of the lead compound was carried out wherein the dilutions of the compound with DMSO, goat anti-C2 pIgG, and goat normal pIgG were tested for inhibition. Each pair of wells received inhibitors at only one stage. After each stage's incubation period, plates were centrifuged at 2400 RPM for 3 minutes, and cell pellets were washed twice with 100 μl/well GVB⁺⁺ to remove inhibitors and unbound protein. EGTA was used in stage 4 to block new addition of C1 from the serum and therefore make the final stage dependent on previous deposition of C3b. In this component assay, anti-C2 pIgG but not normal pIgG, blocked complement activation at stage 3 as expected.

The lead compound inhibited stage 4 in a dose-dependent manner but not stages 1, 2, or 3. These results indicated that the compound did not inhibit formation or activity of the CCP/LCP C3 convertase but inhibited either the C5 convertase or subsequent formation of MAC, the terminal component of the complement system.

In order to determine whether the lead compound inhibited the activity of the C5 convertase or subsequent formation of the MAC, a simplified component assay was carried out. C2-depleted NHS was obtained (Advanced Research Technologies, San Diego, Calif.). EA were washed twice with GVB⁺⁺ and resuspended at 2×10⁹ cells/ml in GVB⁺⁺. An equal volume of GVB⁺⁺ containing a 1:50 dilution of C2-depleted NHS was added and the cells were incubated at 30° C. for 7.5 minutes to allow deposition and activation of C1, and subsequent cleavage of C4. The cell suspension was diluted 20-fold with GVB⁺⁺ to stop the reaction, and centrifuged 2400 RPM for three minutes. The cell pellet was washed three times with GVB⁺⁺ and resuspended at 2×10⁸ cells/ml in GVB⁺⁺. Fifty μl/well of the treated EA was added to duplicate wells of a Costar 96-well round-bottom plate, along with 50 μl/well of GVB⁺⁺ containing 1 μg/ml C2, 50 μg/ml C3, and 1 μg/ml C5, with or without anti-C2 (80 μg/ml) normal goat IgG (80 μg/ml). Lead compound (80 μM) or DMSO was added and the plate was incubated at 30° C. for 20 minutes. Two hundred μl/well of GVB⁺⁺ was added, the plate was centrifuged 2400 RPM, 3 minutes, the supernatants were aspirated, and the pellets washed once with 200 μl/well GVB⁺⁺. The cell pellets were resuspended in 100 μl/well GVB and 100 μl/well GVB containing 40 mM EDTA and 1:50 NHS was added, after which the plate was incubated at 37° C. for 60 minutes. The plate was centrifuged again, and 100 μl/well was transferred to an Immulon4 96-well flat-bottom plate and absorbance determined at 420 nm.

Both the anti-C2 pIgG and the lead compound specifically inhibited hemolysis, indicating that the compounds inhibit the CCP/LCP C5 convertase activity directly. These results were consistent with a potential mechanism of complement inhibition wherein the test compound bound C2 or Factor B and inhibited a conformational change necessary for the serine protease domain to gain access to the C5 substrate. Crystal structure data of the Factor B serine protease domain and modeling of its interaction with the A domain is consistent with this hypothesis [Hua Jing, et al., EMBO J. 19:164-173 (2000)].

The top 5 inhibitors of complement proteins C2 and Factor B are shown in Table 4.

TABLE 4

| | |
|---|---|
| AO | 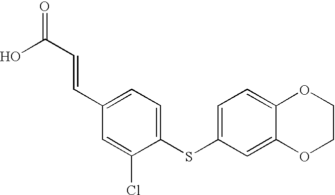 |
| AP | 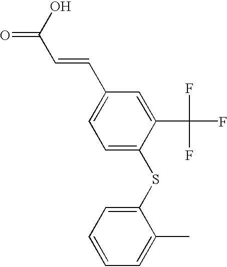 |
| AQ | 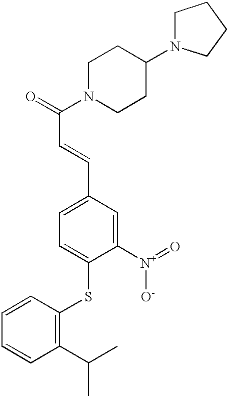 |
| AR | 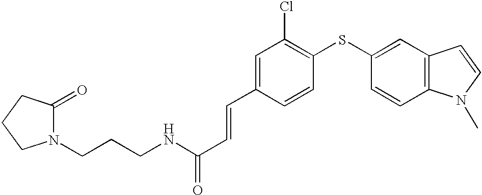 |
| AS | 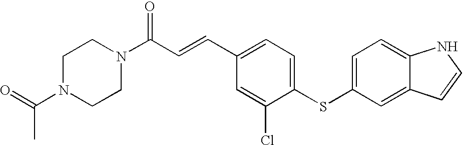 |

### EXAMPLE 5

### Isolation of cDNAs for Alpha E, E-cadherin, and MAdCAM-1

In order to assess whether it is possible to modulate binding activity of other α/β proteins, DNA encoding alpha E, E-cadherin and MAdCAM-1 were prepared as follows.

A. Alpha E

1. Isolation of Human Alpha-E cDNA

DNA encoding human alpha-E was isolated from a normal human intestinal cDNA library (Clontech Laboratories, Inc., Palo Alto, Calif.) using an alpha E I domain cDNA as a probe. The alpha E I domain probe was cloned by PCR amplification using a human colon cDNA library as template and primers encompassing the 5′ and 3′ ends of the alpha E I domain. In order to facilitate cloning, BamHI and XhoI restriction sites (underlined in the sequence) were designed into the 5′ (SEQ ID NO: 7) and 3′ (SEQ ID NO: 8) primers.

| | |
|---|---|
| ATT GGA TCC GCT GGC ACC GAG ATT GCC ATC | SEQ ID |
| | NO:7 |
| AAT TTC TC GAG GTC TCC AAC CGT GCC TTC C | SEQ ID |
| | NO:8 |

A 607 bp I domain fragment was amplified, digested with BamHI and XhoI, and inserted into the plasmid pBluescript® SK (Stratagene, La Jolla, Calif.). The plasmid was transformed into bacteria, plasmid DNA was prepared according published procedures, and the BamHI/XhoI insert was purified. The fragment encoding the alpha E I domain was radiolabeled with ³²P-dCTP and ³²P-dTTP using a random primed DNA labeling kit (Roche Diagnostics Corp., Indianapolis, Ind.) for use as a hybridization probe.

DNA encoding full-length alpha E was identified as follows. A human intestinal cDNA library in phage lambda GT11 (CLONTECH Laboratories, Inc., Palo Alto, Calif.) was plated and hybridized with the I domain probe using standard procedures. From two rounds of screening, six phage clones were isolated. The cDNA inserts were isolated from the phage by EcoRI digestion, subcloned into pBluescript® SK (Stratagene, La Jolla, Calif.), and sequenced. A complete 3.4 kb sequence was reconstituted from three different clones: clone A (3) encompassing the 5′ end, clone B (22) that included sequences from in the middle of the cDNA, and clone C (22) encompassing the 3′ end of alpha E cDNA. Sequence analysis indicated that clone A (3) contained an insertion of two cytidines and another insertion of a guanine at positions 357 and 464, respectively, when compared to the published nucleotide sequence. These insertions resulted in a 75 base frameshift in the open reading frame which resulted in the addition of 25 additional amino acid residues, shown below, not found in the previously reported sequence.

PKGRHRGVTVVRSHHGVLICIQVLVRR SEQ ID NO: 9

The sequences downstream from this 25 amino acid insertion were identical to the published alpha E sequence for the rest of the molecule.

In order to subclone the alpha E cDNA into pcDNA3® (Invitrogen Corp., Carlsbad, Calif.), a HindIII site was generated at the 5′ end by PCR amplification using the 5′ primer Eo26-H3 (SEQ ID NO: 10) and the 3′ primer Eo-24 (SEQ ID NO: 11) primers shown below.

| | |
|---|---|
| GAG GGG AAG CTT AGT GGG CC | SEQ ID NO:10 |
| GAA GTT GGC CTG AGC CTG G | SEQ ID NO:11 |

The PCR product was digested with Hind III and NsiI, and ligated into the corresponding sites of the vector.

The expression vectors pMHneo [Hahn et al., Gene 127:267-268 (1993)] and pcDNA3®/aE were transformed into the bacterial strain NEB316, a dam⁻ strain which does not methylate XbaI restriction sites, and plasmid DNA isolated according to standard procedures. Both pMHneo and pcDNA3®/aE were digested with HindIII and XbaI and the 3.4 kb alpha E cDNA fragment from pcDNA3®/aE was separated using agarose gel electrophoresis. The fragment was excised from the gel, purified, and ligated into HindIII/XbaI-digested vector pMHneo. An aliquot of ligation mixture was used to transform XL-1 Blue bacteria (Stratagene, La Jolla, Calif.) according to the manufacturer's protocol, and bacterial colonies containing pMHneo were selected by growth on LBM agar plates containing ampicillin. Bacterial colonies were grown overnight in LBM media containing 100 ug/ml ampicillin and plasmid DNA was isolated using the Wizard Plus Miniprep Kit (Promega Corp., Madison, Wis.). The plasmid DNA was characterized by diagnostic restriction digestion and a plasmid containing the alpha E cDNA, referred to as pMHneo/aE, was used to stably transfect a JY cell line as described below.

B. E-cadherin

1. Isolation of E-cadherin cDNA

The cDNA for human E-cadherin was isolated by PCR amplification of a Marathon-Ready™ human colon cDNA library (CLONTECH Laboratories, Inc. Palo Alto, Calif.) using E-cad 5′#1 (SEQ ID NO: 12) and E-cad 3′#1 (SEQ ID NO: 13) primers, which are set forth below.

| | |
|---|---|
| 5′-CTGCCTCGCTCGGGCTCCCCGGCCA-3′ | SEQ ID NO:12 |
| 5′-CTGCACATGGTCTGGGCCGCCTCTCTC-3′ | SEQ ID NO:13 |

Polymerase chain reactions were performed in a Perkin Elmer Cetus (PE Applied Biosystems, Foster City, Calif.) DNA thermal cycler in a reaction mixture containing 5 μl of the library cDNA, 10 μl of 5×PCR buffer from an Advantage™-GC cDNA PCR Kit (CLONTECH Laboratories, Inc. Palo Alto, Calif.), 1 μl of 50×dNTP mix, 1 μl of 10 μM primer E-cad5′#1, 1 μl of 10 μM primer E-cad 3′#1, 1 μl of Advantage™ KlenTaq polymerase mix, and 31 μl of H₂O. Amplification conditions included an initial incubation for 1 min at 94° C., followed by 5 cycles at 94° C. for 30 sec and 72° C. for 4 min; 5 cycles at 94° C. for 30 sec and 70° C. for 4 min; 25 cycles at 94° C. for 30 sec and 68° C. for 4 min; and a final 5 min incubation at 72° C. An aliquot of the reaction was separated using agarose gel electrophoresis to determine the approximate size of the PCR product and a single band of ˜2.7 kb was detected as anticipated. The 2.7 kb PCR product was ligated into the plasmid pCR®2.1 using a TA Cloning® Kit (Invitrogen Corp., Carlsbad, Calif.) according to the manufacturer's protocols. E. coli strain INVaF′ (Invitrogen Corp., Carlsbad, Calif.) was transformed with an aliquot of the ligation reaction as recommended by the manufacturer and single bacterial colonies were isolated and grown overnight in LBM media containing 100 μg/ml ampicillin. Plasmid DNA was isolated from these cultures using the Wizard Plus Miniprep Kit (Promega Corp., Madison, Wis.).

2. Generation of DNA Encoding a E-cadherin/Ig Fusion Protein

The extracellular region of E-cadherin is made up of five tandem repeats (domains) of approximately 110 amino acids each. In order to express an E-cadherin-human/human IgG1 fusion protein, a DNA fragment containing domains 1 through 5 of E-cadherin was generated by PCR amplification of the E-cadherin cDNA (pCR®2.1/E-cadherin #3 described above) with primers Ecad5′Kozak (SEQ ID NO: 14) and Ecad3′(Xho) (SEQ ID NO: 15). The 5′ primer Ecad5′Kozak was used to add a 5′ HindIII site to facilitate subsequent subcloning of the 5-domain fragment into the expression vector pDEF2 (see U.S. Pat. No. 5,888,809) and reconstitute a Kozak sequence upstream of the translation initiation codon which was lacking from initial E-cadherin cDNA clone. The 3′ primer Ecad3′(Xho) generated a new 3′ end of the fragment containing domains 1 through 5 of E-cadherin, and added a XhoI restriction site to the 3′ terminus of the fragment to facilitate subsequent subcloning of the 5-domain fragment into pDEF2.

| | |
|---|---|
| 5′-GCGTTAAAGCTTCACAGCTCATCACCATGGGCCCTTGGAGCCGCA-3′ | SEQ ID NO:14 |
| 5′-AGGCGCTCGAGAATCCCCAGAATGGCAGGAATT-3′ | SEQ ID NO:15 |

The E-cadherin cDNA fragment contained in pCR2.1/E-cad#3 was amplified by PCR in a reaction containing 0.5 μl of pCR2.1/E-cad#3, 10 μl of 5×PCR reaction buffer, 1 μl of 10 μM primer Ecad5′Kozak, 1 μl of 10 μM primer E-cad3′(Xho), 1 μl of Advantage™ KlenTaq polymerase mix, and 35.5 μl of H₂O. Amplification conditions included an initial incubation for 1 min at 94° C.; 5 cycles at 94° C. for 30 sec and 72° C. for 4 min; 5 cycles at 94° C. for 30 sec and 70° C. for 4 min.; 25 cycles at 94° C. for 30 sec and 68° C. for 4 min; and a final 5 min incubation at 72° C. An aliquot of the PCR reaction was resolved by agarose gel electrophoresis, and a single band of 2.1 kb was observed as expected. The fragment was purified using the Wizard PCR Purification Kit (Promega Corp., Madison, Wis.), and digested with XhoI and HindIII under standard conditions. The resulting fragment was referred to as 5′-HindIII-Kozak-E-cadherin-XhoI-3′.

The plasmid pDC1/ICAM3.IgG1 was digested with XbaI and SalI and a fragment of 908 bp (referred to as 5′-SalI-IgG1-XbaI-3′) with a 5′ terminal SalI site and a 3′ terminal XbaI site was purified from a low melting temperature agarose gel (FMC BioProducts, Rockland, Me.). This fragment contains the sequences encoding the CH2-CH3 region of human IgG1. The expression vector pDEF2 was linearized in the multiple cloning site with HindIII and XbaI and a three-way ligation reaction was performed which contained the 5′-HindIII-Kozak-E-cadherin-XhoI-3′ fragment, linearized pDEF2, and the 5′-SalI-IgG1-XbaI-3′ fragment. In this reaction, the 3′ XhoI site in 5′-HindIII-Kozak-E-cadherin-XhoI-3′ was joined in-frame to the 5′-SalI-IgG1-XbaI-3′; both XhoI and SalI have compatible 5′ overhangs which can be ligated together but cannot be re-digested with either XhoI or SalI. An aliquot of the ligation reaction was used to transform the bacterial strain XL-1 Blue (Stratagene, La Jolla, Calif.). Individual bacterial colonies were grown overnight in LBM containing 100 μg/ml ampicillin, and plasmid DNA was isolated with a Wizard Plus Miniprep Kit (Promega Corp., Madison, Wis.). The pDEF2/E-cadIgG1 plasmid DNA was digested with HindIII and XbaI and the digestion products resolved by agarose gel electrophoresis. Those clones containing a 2.1 kb fragment were sequenced to ensure that the E-cadherin-IgG1 chimera maintained an open reading frame across the E-cadherin/IgG1 junction.

The pDEF2/E-cadIgG1 clone #3 was found to contain a continuous open reading frame across the E-cadherin/IgG1 junction and was used for CHO cell expression studies described below. The open reading frame of the E-cadherin/IgG1 fusion was not sequenced in its entirety since the DNA fragments contributing to this chimera had been previously sequenced and had not been subjected to PCR amplification.

C. MAdCAM-1-1

1. Isolation of a Partial cDNA for Human MAdCAM-1-1.

A fragment containing a partial cDNA for MAdCAM-1 was isolated by PCR amplification of Marathon-Ready™ human spleen cDNA library with an Advantage™-GC cDNA PCR Kit (CLONTECH Laboratories, Inc., Palo Alto, Calif.). Polymerase chain reactions were performed in a Perkin Elmer Cetus (PE Applied Biosystems, Foster City, Calif.) DNA thermal cycler in a reaction containing 5 μl of Marathon™ human spleen cDNA, 1 μl of 10 μM primer MAdCAM-1 5′#1 (SEQ ID NO:16), 1 μl of 10 μM primer MAdCAM-1 3′#5 (SEQ ID NO: 17), 10 μl of 5.0 M GC-Melt™, 1 μl of 50×dNTP mix, 1 μl of Advantage™ KlenTaq polymerase mix, 10 μl of 5×reaction buffer, and 21 μl of H₂O.

| | |
|---|---|
| 5′-ATGGATTTCGGACTGGCCCTCCTGCT-3′ | SEQ ID NO:16 |
| 5′-CTCCAAGCCAGGCAGCCTCATCGT-3′ | SEQ ID NO:17 |

Amplification conditions included an initial incubation for 1 min at 94° C.; 5 cycles at 94° C. for 30 sec and 72° C. for 3 min; 5 cycles at 94° C. for 30 sec and 70° C. for 3 min; 25 cycles at 94° C. for 30 sec and 68° C. for 3 min; and a final incubation for 5 min at 68° C. An aliquot of the reaction was resolved by agarose gel electrophoresis and a single fragment of 640 bp was detected. The fragment was subcloned into pCR®2.1 and amplified in bacteria using the TA Cloning® Kit (Invitrogen Corp., Carlsbad, Calif.) following the manufacturer's protocol. Single bacterial colonies were grown overnight in LBM containing 100 μg/ml ampicillin. Plasmid DNA was isolated from the cultures using a Wizard Plus Miniprep Kit (Promega Corp., Madison, Wis.), and the nucleotide sequence of the subcloned PCR product was determined by DNA sequence analysis. This partial cDNA for MAdCAM-1 begins with the initiation codon and terminates at its 3′ end at residue 640 in domain 2. The sequence of this partial MAdCAM-1 cDNA is identical to that previously reported [Shyjan et al., J. Immunol. 156:2851-2857 (1996)].

2. Additional PCR Amplification DNA Encoding MAdCAM-1 Domains 1 and 2

In order to express domains 1 and 2 of MAdCAM-1 as a secreted immunoglobulin fusion protein, it was essential to: (i) restore a Kozak sequence upstream of the initiation codon to allow for efficient protein translation; (ii) add a 5′ HindIII site to facilitate subcloning of the fragment into pDEF2; (ii) extend the open reading frame of the existing partial MAdCAM-1 cDNA to encompass additional amino acid residues needed to encode the entire second domain; and (iv) introduce a SalI site at the 3′ terminus of the fragment to facilitate subcloning into pDEF2. These modifications were introduced into the MAdCAM-1 fragment described above by PCR amplification using the primers Mad5′Kozak (SEQ ID NO: 18) and Mad 3′ #6 Sal (SEQ ID NO: 19) as shown below.

| | |
|---|---|
| 5′-GCGTTAAAGCTTCACAGCTCATCACCATGGATTTCGGACTGGCCCTCCT-3′ | SEQ ID NO:18 |
| GCTAGTCGACGGGGATGGCCTGGCGGTGGCTGAGCTCCGAAGCAGGCAGCCTCATCGT | SEQ ID NO:19 |

The PCR reaction included 0.5 μl of pCR®2.1/MAd#4-1 template DNA, 10 μl of 5×PCR buffer, 10 μl of 5.0 M GC Melt™, 1 μl of 50×dNTP mix, 1 μl of 10 μM, 1 μl of 10 μM, 1 μl of 50×Advantage™ KlenTaq polymerase mix, and 25.5 μl of H₂O. The PCR amplification conditions included 94° C., for 1 min; 5 cycles at 94° C. for 30 sec and 72° C. for 2 min; 5 cycles at 94° C. for 30 sec and 70° C. for 2 min; 20 cycles at 94° C. for 30 sec and 68° C. for 2 min; and 68° C. for 5 min. An aliquot of the reaction was resolved by agarose gel electrophoresis and a single fragment of ˜0.7 kb was detected as expected. The PCR product was purified using the Wizard PCR Purification Kit (Promega Corp., Madison, Wis.) and digested with HindIII and SalI under standard conditions. The fragment was ligated into HindIII/SalI digested pBluescript® SK plasmid DNA (Stratagene, La Jolla, Calif.) under standard conditions, and the sequence of the MAdCAM-1 fragment in pBS-SK/Mad#7 was determined.

3. Generation of MAdCAM-1/Ig Fusion Protein

To generate an expression vector encoding a chimeric domain1/domain2 MAdCAM-1-IgG1 fusion protein, the 702 bp HindIII-SalI fragment from pBS/Mad#7, the 908 bp SalI-XbaI fragment from pDC1/ICAM3.IgG and pDEF2 linearized by digestion with HindIII and XbaI were combined in a ligation reaction. An aliquot from the ligation reaction was used to transform XL-1 Blue bacteria (Stratagene, La Jolla, Calif.) and the plasmid DNA isolated from single colonies were screened by restriction digestion with HindIII, XbaI, and SalI. One clone, pDEF2/MadIg#1, was found to contain all three fragments and was used to generate stably transfected CHO cell lines as described below.

### EXAMPLE 6

### Expression of MAdCAM-1/Ig and E-cadherin/Ig

A. Generation of Stable CHO Cell Lines Expressing MAdCAM-1/Ig and E-cadherin/Ig

For transfection of host CHO DG44 cells with pDEF2/MAdIg or pDEF2/EcadIg, 50 to 100 ug of plasmid was linearized by digestion with the restriction enzyme PvuI. DG44 cells were cultured in DMEM/F-12 medium supplemented with hypoxanthine (0.01 mM final concentration) and thymidine (0.0016 mM final concentration), also referred to as “HT”. DG44 cells were prepared for transfection by growing cultures to about 50% or less confluency in treated 150 cm² tissue culture polystyrene flasks (Corning Inc., Corning, N.Y.). Cells were collected and resuspended in 0.8 ml of a solution containing HeBS buffer (20 mM Hepes, pH 7.0, 137 mM NaCl, 5 mM KCl, 0.7 mM, Na₂HPO₄ and 6 mM dextrose) with the desired plasmid DNA. The resuspended cells were electroporated at room temperature with a capacitor discharge of 290 V and 960 μF (9 to 11.5 msec pulse). Cells were added to 10 ml DMEM/F-12 supplemented with 5% dialyzed FBS and HT, pelleted by centrifugation, resuspended in 2 ml DMEM/F-12 supplemented with 5% dialyzed FBS and HT (“non-selective media”), and seeded into 75 cm² polystyrene tissue culture flasks. After two days growth the cells were collected and seeded at varying dilutions in DMEM/F-12 supplemented with 5% dialyzed FBS and without HT (“selective media”).

Once selection was complete and single cell clones could be identified, a single cell suspension of pooled CHO transfectants was prepared by typsinization. In order to isolate individual clones, the CHO/MAdCAM-1Ig and CHO/E-cadIg transfectants were plated at a density of approximately 1 cell/well in Immulon-4 96-well plates (Dynex Technologies, Inc., Chantilly, Va.) under selective conditions. Once single colonies were detected in the 96-well plates, supernatant from each well was screened for the presence of MAdCAM-1 /Ig or E-cadherin/Ig fusion protein. Single cell CHO clones producing a human IgG1 protein component were expanded, and those clones producing the greatest level of MAdCAM-1 /Ig or E-cadherin/Ig fusion protein were selected for large-scale protein production.

In large-scale protein production, the CHO/MadIg and CHO/E-cadIg clones were expanded in serum-free 5.2 (HT⁻) media in a spinner flask maintained at 37° C. in an atmosphere of 5% CO₂. When cell densities exceeded 10⁶ cells/ml., the media was harvested and the spinner flask was provided with fresh 5.2 (HT−) media. The spent media was first centrifuged to remove cell debris, filtered through a 0.22 μm 1 liter filter unit (Corning Inc., Corning, N.Y.), and stored at 4° C.

B. MAdCAM-1/Ig Purification

MAdCAM-1/Ig was purified by affinity chromatography using a protein A-Sepharose® 4 Fast Flow resin column (Flow (Amersham Pharmacia Biotech, Inc., Piscataway, N.J.) equilibrated with CMF-PBS. The cell supernatant was cycled through the column at a rate of 4 ml/min. After loading, the column was washed with CMF-PBS until there was no detectable protein present in the eluate. MAdCAM-1/Ig was eluted with 0.1 M acetic acid (pH 3.0) into a tube containing 1M Tris, pH 9.0, and the sample was dialyzed at 4° C. against CMF-PBS.

C. Purification of E-cadherin/Ig

E-cadherin/Ig was purified by affinity chromatography using a protein A-Sepharose® 4 Fast Flow resin column (Amersham Pharmacia Biotech, Inc., Piscataway, N.J.) equilibrated with D-PBS. The supernatant was cycled through the column at a rate of approximately 4 ml/min. After loading, the column was washed with Tris-buffered saline, pH 8.0, containing 1 mM CaCl₂ until there was no detectable protein present in the eluate. E-cadherin/Ig was eluted with 0.1 M acetic acid (pH 3.0) containing 1 mM CaCl₂ into a tube containing 1M Tris, pH 9.0. Calcium concentration was adjusted to 1 mM and the sample was dialyzed at 4° C. against Tris-buffered saline (pH 6.8) containing 1 mM CaCl₂.

### EXAMPLE 7

### Generation of JY/alpha-E Transfectants

The human B lymphoblastoid cell line, JY, was transfected with the plasmid pMHneo/aE as described above. The transfected population was grown in “selection media” (containing RPMI 1640 media supplemented with 5% FBS, 100 U/ml penicillin G, 100 μg/ml streptomycin sulfate, 2 mM L-glutamine, 1 mM sodium pyruvate, and 1.0 mg/ml G418) and after 14 days, 10⁸ G418-resistant JY cells were resuspended in 5 ml of selection media containing 5 μg/ml of the anti-aE monoclonal antibody Ber-ACT8 (DAKO Corp., Carpinteria, Calif.) and incubated on ice for 1 hour. Cells were collected by centrifugation, and the media was aspirated. The JY/aE transfectants were stained with selection media containing a 1:200 dilution of sheep anti-mouse Ig-FITC (Sigma Corp., St. Louis, Mo.) on ice for 1 hour. Unbound antibody was removed by centrifugation and the supernatant aspirated. Alpha E-expressing cells were isolated by flow cytometry and subsequently expanded by in vitro culture in selection media.

Re-analysis of the sorted JY/aE⁺ population by flow cytometry with Ber-ACT8 revealed a bimodal population of cells that contained both alpha E-expressing and alpha E-nonexpressing cells. The bimodal population was stained a second time with Ber-ACT8 as previous described, and individual JY/aE cells were sorted into a 96-well Immulon-4 plate containing selection media. Single cell JY/aE⁺ clones expressing high levels of alpha E were expanded in vitro and JY/aE clone #47 was selected for further characterization. This clone, but not the parental JY cells, displayed robust adhesion to recombinant E-cadherin/Ig and the binding was induced with phorbol ester treatment of the cells. Binding of JY/aE clone #47 to E-cadherin/Ig was blocked by the anti-β₇ integrin antibody FIB504 (ATCC, Rockville, Md.), as well as by antibodies to E-cadherin (Zymed Corp., So. San Francisco, Calif.).

### EXAMPLE 8

### Isolation of a JY/alpha D⁺ Clones

To obtain a JY cell line that stably expresses the α_{d}β₂ integrin, JY cells were electroporated with pMHneo/aD as described above and stable transfectants were selected by growth in selection media. After a G418-resistant population of cells had been selected, JY/aD⁺ cells were stained with the anti-α_{d} monoclonal antibody 212D and sheep anti-mouse-FITC (Sigma Corp., St. Louis, Mo.). Single cell JY/aD⁺ clones were isolated by cell sorting using a flow cytometer as previously described for the isolation of single cell JY/aE⁺ clones.

### EXAMPLE 9

### JY/aE⁺ Adhesion Assays

A. Compound Dilutions

Adhesion media (350 μl) (RPMI 1640 containing penicillin and streptomycin, L-glutamine, NaPy, and 5% FBS) was aliquotted into each well in rows A, C, E, G of a deepwell 96-well titer plate, 2.0 ml capacity (Beckman Instruments, Inc., Fullerton, Calif.) in columns 1-11. All compounds to be screened were dissolved in DMSO to a final concentration of 10 mM. Compounds were stored at −20° C., and thawed on the day of use in a 37° C. incubator. Each compound (2.1 μl) was pipetted into a single well in columns 3-11, rows A, C, E, and G in the deepwell titer plate. To wells not containing compound (A1 & A2, C1 & C2, E1 & E2, G1 & G2), 2.1 μl DMSO was added. An anti-β₇ monoclonal antibody, FIB504 (ATCC, Rockville, Md.), which blocks α_{E}β₇ binding activity, was added to wells C2 and G2 at a concentration of 7.5 μg/ml. Each deepwell titer plate was covered to prevent dessication and stored in a 37° C. incubator until ready for use.

B. Adhesion Assay

Adhesion assays were performed in 96-well Immunlon 4 plates (Dynex Technologies, Inc., Chantilly, Va.) as follows. Each well was coated with 50 μl E-cadherin/Ig (3.0 μg/ml) in D-PBS. Control wells were coated with capture antibody FIB504, to quantitate 100% input cell binding, or coating buffer alone to determine background binding. Following an overnight incubation at 4° C., the plates were washed three times with 200 μl/well D-PBS and blocked with 1% BSA in D-PBS for at least 1 hour. The BSA solution was removed and 100 μl of adhesion media (RPMI 1640 containing penicillin and stretomycin, L-glutamine, sodium pyruvate, 0.1% BSA, and 60 ng/ml PMA), was added to rows B through G, columns 1 through 11.

At this point, 100 μl adhesion media containing a test compound at a concentration of 60 μM, was transferred from the deepwell 96-well titer plate, in triplicate, to the E-cadherin/Ig coated adhesion plate. The outer rows were filled with 300 μl of D-PBS. These plates were transferred to a humidified 37° C. incubator with an atmosphere of 5% CO₂.

The adhesion assay was initiated by addition of 100 μl of the JY/aE⁺ cell suspension to each well of the E-cadherin-coated plate. The final volume in each well was 300 μl adhesion media containing 105 cells, PMA (final concentration 20 ng/ml), and the test compound (final concentration 20 μM). The plates were incubated at 37° C. for 30 min. Each compound was tested in triplicate.

Adherent cells were fixed by the addition of 50 μl of a 14% glutaraldehyde solution in D-PBS. Plates were washed with water, stained with 100 μl/well 0.5% crystal violet (Sigma Corp., St. Louis, Mo.) solution for 5 min. Three hundred microliters/well of 70% ethanol was added, and adherent cells were quantitated by determining absorbance at 570 nm. Percentage of cell binding was determined by using the mean values for each triplicate in a given assay in the following formula.

$$\% binding = \frac{\begin{matrix}( A570 ( binding to E \text{-} cadherin / Ig ) - \\ A570 ( binding to BSA )\end{matrix}}{\begin{matrix}A570 ( binding in adhesion media \\ without compound )\end{matrix}} \times 100$$

C. IC50 Determinations

During the initial screening of test compounds, each chemical entity was tested in cell-based adhesion assays at a fixed concentration of 20 μM. Those compounds that blocked JY/α_{E}β₇-dependent adhesion to E-cadherin/Ig by 50% or more were subsequently retested at multiple concentrations to determine the inhibitory concentration at which cell binding is reduced by 50%, i.e., the IC50 value.

Of the compounds screened, 40, or 1.4 % of the total library, inhibited α_{E}β₇-E-cadherin adhesion by 40% or greater. Approximately 18 of the compounds were identified in the diarylamide library, and 22 compounds were identified in the diaryl sulfide library. Upon re-analysis of these primary hits in IC₅₀ determinations, 4 of the 40 compounds were shown to inhibit JY/aE+ binding to E-cadherin with an IC₅₀ value of not more than 10 μM. Many of the initial hits were eliminated from further characterization if their initial inhibitory activity was not reproducible; or a compound was shown to inhibit multiple integrin-dependent adhesive events; or the IC₅₀ value exceeded 10 μM, or it displayed any cytopathic or cytotoxic effects. The following compounds displayed reproducible inhibitory activity at compound concentrations below 10 μM: Cmpd K, Cmpd W, Cmpd Z, Cmpd D as set out in Table 2. There were several compounds that displayed significant inhibitory activity in the initial screen that failed to inhibit JY/aE+/E-cadherin binding upon re-analysis. It is possible that the activity of some diaryl compounds was lost upon repeated freezing and thawing.

To assess the selectivity of each compound, an IC50 value was determined for additional binding partner compounds JY/α_{V}β₃ and vitronectin, JY/α₄β₁ and VCAM/Ig, JY/α_{d}β₂ and VCAM, JY/α_{L}₂ and ICAM-1, JY/α_{M}β₂ and iC3b, and JY/α₄β₇ and MAdCAM-1.

For each IC50 assay, 50 μl of the ligand diluted in 50 mM bicarbonate buffer (pH 9.6) was dispensed per well of an Immulon-4 plate. A single plate was used to test two different ligands, each in triplicate, The coating concentration for the various ligands was as follows: VCAM-1/Ig at 2.0 μg/ml; ICAM-1/Ig at 5.0 μg/ml; vitronectin at 0.5 μg/ml; MAdCAM-1/Ig at 3.0 μg/ml, and iC3b at 5.0 μg/ml. The capture antibody, e.g. anti-CD18 monoclonal antibody TS1/22, was added at a concentration of 10 μg/ml in 50 μl/well. Ligand-coated plates were covered and stored overnight at 4° C. The following day, the contents of each well was decanted, and each plate was washed three times with 200 μl/well D-PBS. The plate was then blocked by the addition of 300 μl/well of 1% BSA/D-PBS solution. Each plate was again covered and incubated at room temperature for at least 1 hour.

For each IC50 determination, the test compound was serially diluted in DMSO to enable testing at final concentrations of 40 μM, 20 μM, 10 μM, 5.0 μM, 2.5 μM, 1.25 μM, 0.63 μM, 0.32 μM and 0.16 μM. Prior to transfer to the adhesion plate, the compounds were initially diluted by transferring 4.2 μl of the diluted compounds to a 96-well deepwell titer plate containing 0.7 ml/well of RPMI 1640, 0.1% BSA, and 3 ng/ml PMA (Sigma Corp., St. Louis, Mo.) pre-warmed to 37° C. The 1% BSA/D-PBS blocking solution was decanted from the 96-well Immulon-4 plates and replaced with 0.2 ml of diluted compound. For each 96-well plate to be screened, approximately 8×10⁶ cells were collected by centrifugation and resuspended in adhesion media (RPMI 1640 containing 0.1% BSA) to a final concentration of 10⁶/ml. To prevent PMA-dependent homotypic aggregation in the adhesion assay, the anti-CD18 antibody 22F12C (ICOS Corp., Bothell, Wash.) was added to the cell suspensions to a final concentration of 10 μg/ml, and the cells were incubated at 37° C. for 15 min. This antibody was not added to CD18-dependent adhesion assays involving JY/α_{L}β₂, JY/α_{d}β₂ or JY/α_{M}β₂ and their corresponding ligands ICAM-1, VCAM-1, or iC3b.

The adhesion assay was initiated by addition of 100 μl of the cell suspension to each well of the Immulon-4 plate. The plates were incubated at 37° C. for 30 min and adherent cells were fixed for least 1 hour by the addition of 50 μl of a 14% glutaraldehyde solution in D-PBS. The plates were washed with water and stained with 100 μl/well 0.5% crystal violet (Sigma Corp., St. Louis, Mo.) solution for 5 min. The plates were washed a second time with water to remove excess crystal violet dye, and 300 μl 70% ethanol was added to each well. Adherent cells were quantitated by determining the absorbance at 570 nm in a plate spectrophotometer. The percentage of cell binding was determined by using the mean values for each triplicate in a given assay and the formula below.

$$\% Binding = \frac{\begin{matrix}A570 ( binding to ligand ) - \\ A570 ( binding to BSA )\end{matrix}}{\begin{matrix}A570 ( binding in adhesion \\ media without compound )\end{matrix}} \times 100$$

The four compounds Cmpd K, Cmpd W, Cmpd Z, Cmpd D identified in the primary screen were selected for further specificity profiling, whereby their IC₅₀ values were determined in additional integrin-dependent adhesive events. In all cases, the indicator cell line used in the binding assay was treated with 2 ng/ml PMA during the course of the assay to stimulate integrin-dependent adhesion. The IC50 values of these four compounds were determined in adhesion assays as indicated in Table 5.

TABLE 5

| | E-cadher | MAdCAM | iC3b | VN | ICAM-1 | VCAM | VCAM |
|---|---|---|---|---|---|---|---|
| Compound | α_{E}β₇ | −1 α₄β₇ | α_{M}β₂ | α_{V}β₃ | α_{L}β₂ | α₄β₁ | α_{d}β₂ |
| Cmpd K | 3 μM | 4 μM | 4 μM | 6 μM | 11 μM | >40 μM | >40 μM |
| Cmpd D | 3 μM | 4 μM | 7 μM | 4 μM | 28 μM | >40 μM | >40 μM |
| Cmpd W | 5 μM | 5 μM | 4 μM | 8 μM | 30 μM | >40 μM | >40 μM |
| Cmpd Z | 3 μM | 11 μM | ND | ND | 20 μM | >40 μM | >40 μM |

### EXAMPLE 10

### Cloning, Expression and Purification of Alpha 1, Alpha 2 and Alpha 11 I Domains

The collagen-binding integrins alpha 1, alpha 2 and alpha 11 contain I domain sequences homologous with the I domain sequences contained in the leukointegrins alpha L, alpha M, alpha X and alpha d. To investigate the possibility that these molecules might be susceptible to modulation through an allosteric regulatory site, the library of test compounds was assessed for the ability to inhibit interactions between these integrins and their ligands collagen and laminin.

The alpha 1 and alpha 2 I domain sequences and alpha 11 were cloned into the bacterial expression vector pET15b (Novagen). Expression of these constructs in E. coli results in proteins with an amino terminal histidine tag and the “tagged” protein which can be purified using a nickel column. The cloning of the alpha 11 was carried out as previously described [Veiling, et al., J. Biol. Chem. 274:25735-25742 (1999)].

Both alpha 1 and alpha 2 I domain sequences were cloned into pET15b following PCR amplification to add restriction sites that permit the I domains to be cloned in frame with the histidine tag in the vector. The template for the alpha 1 I domain PCR reaction was a full-length alpha 1 cDNA cloned by hybridization from a spleen cDNA library in vector pcDNA-1 Amp as previously described. The hybridization probe used for this screen was the product of the PCR reaction using the following Alpha1.5 (SEQ ID NO: 20) and Alpha1.3 (SEQ ID NO: 21) primers, respectively:

| | |
|---|---|
| 5′-GACTTTCAGCGGCCCGGTGGAAGACATG-3′ | SEQ ID NO:20 |
| 5′-CCAGTTGAGTGCTGCATTCTTGTACAGG-3′ | SEQ ID NO:21 |

The samples were initially incubated at 94° C. for 30 sec followed by 5 cycles of 94° C. for 5 sec and 72° C. for 2 min; 5 cycles of 94° C. for 5 sec and 70° C. for 2 min; 25 cycles of 94° C. for 5 sec and 68° C. for 2 min; and a final incubation of 72° C. for 7 min. The PCR products were cloned into the TOPO TA vector pCRII (Invitrogen) and sequenced. The resulting clone was used as a template in PCR using the same conditions as above and the amplification product was gel purified, labeled with ³²P using a random primed labeling kit (Boehringer Mannheim), and used as a hybridization probe. Hybridization was performed using ExpressHyb hybridization solution (Clontech) under the same conditions used in the screening for full length alpha 11 cDNA The resulting clone, alpha1/pcdna/111 was used as a template to subclone the alpha 1 I domain.

The alpha1 I domain was amplified by PCR using A1.5Nde (SEQ ID NO: 22) and A1.3Bam (SEQ ID NO: 23) primers, respectively shown below

| | |
|---|---|
| 5′-ATATCATATGGACATAGTCATAGTGCTGG-3′ | SEQ ID |
| | NO:22 |
| 5′-ATATGGATCCCTAAGACATTTCCATTTCAAATG-3′ | SEQ ID |
| | NO:23 |

The alpha 2 I domain was cloned by PCR using a HUVEC cDNA library in the vector pcDNA-1 Amp as template and A2.5Nde (SEQ ID NO: 24) and A2.3Bam (SEQ ID NO: 25) primers, respectively shown below:

| | |
|---|---|
| 5′-ATATCATATGGATGTTGTGGTTGTGTGTG-3′ | SEQ ID |
| | NO:24 |
| 5′-ATATGGATCCCTATGACATTTCCATCTGAAAG-3′ | SEQ ID |
| | NO:25 |

PCR conditions for amplification of both I domains included an initial incubation at 94° C. for 2 min followed by 30 cycles of 94° C. for 20 sec; 55° C for 30 sec and 72° C. for 45 sec; and a final incubation at 72° C. for 7 min. The PCR products were gel purified, digested with NdeI and BamHI, gel purified again, and cloned into pET15b previously digested with same enzymes. The resulting clones alpha1/pet/2 and alpha2/pet/27 were sequenced.

The alpha 1, alpha 2 and alpha 11 pET15b clones were transformed into the bacterial strain BL21 (DE3)pLysS (Stratagene) for expression. Histidine-tagged proteins were isolated from the soluble fraction of the E. coli lysate using a Ni NTA agarose column (QIAGEN) and elution with an imidazole gradient. The eluted proteins were dialyzed against CMF-PBS and biotinylated using EZ-Link Sulfo-NHS-LC-Biotin (Pierce) according to the manufacturer's suggested protocol.

An assay for measuring alpha 1 or alpha 2 I domain binding to collagen in a 96-well plate format involves binding collagen to the wells of a 96-well plate, adding biotinylated alpha 1 or alpha 2 protein to the wells and measuring the amount of collagen bound I domain using europium-coupled streptavidin and time resolved fluorescence. Immulon4 96-well plates were coated with 20 μl/ml of rat type I collagen (Sigma) in CMF-PBS overnight at 4° C. Wells were washed with 250 μl of CMF-PBS two times and blocked with 2.5% BSA in CMF-PBS at 30° C. for 1 hr. The wells were washed with 200 μl of CMF-PBS and biotinylated protein was added to the wells at 1 μg/ml in either CMF-PBS with 2 mM MgCl₂ and 1% BSA or in TBS with 2 mM MnCl₂ and 1% BSA and incubated at 37° C. for 3 hours. The wells were washed with 200 μl of the same incubation buffers (without I domain protein) two times and collagen bound biotinylated protein was detected with the addition of 100 μl of a 1:1000 dilution of streptavidin europium (SA-Eu; Wallac) in SA-Eu dilution buffer (Wallac). Incubation was for 1 hour at room temperature. The wells were washed with 200 μl of incubation buffer six times and 100 μl of Enhancement solution (Wallac; diluted 1:1 with water) was added to each well for 5 minutes at room temperature. Fluorescence was measured using the Eugen program.

### EXAMPLE 11

### Identification of Alpha2 Antagonists of Collagen Binding

FACS analysis has indicated that Jurkat cells express both alpha1 and alpha2 integrins. Binding studies using monoclonal antibodies to each of these integrins has shown that Jurkat cell adhesion to rat type I collagen is mediated predominantly through interaction with alpha2. For example, an alpha2 blocking monoclonal antibody has been shown to completely inhibit Jurkat cell binding to type I collagen. In view of this result, Jurkat cells were employed in an adhesion assay as described below to identify inhibitors of alpha2 binding. The assay was carried out using a modification of a procedure previously described [Sadhu, et al., supra].

Immunlon 4 plates (Dynex Technologies, Chantilly, Va.) were coated overnight at 4° C. with (i) 50 μl rat type I collagen (Sigma) (20 μg/ml in CMF-PBS), (ii) anti-beta1 monoclonal antibody 3S3 (5 μg/ml) in bicarbonate buffer, pH 9.6, (iii) or bicarbonate buffer alone. Plates were washed once with 200 μl/well D-PBS and blocked with 1% BSA (100 μl/well) in D-PBS for 1 hr at room temperature. Wells were rinsed once with 100 μl adhesion buffer containing RPMI and 1% inactivated FBS and 100 μl adhesion buffer containing PMA (10 ng/ml final concentration) was added to each well. Adhesion buffer (100 μl) with or without candidate inhibitor (at a final concentration of 20 μM) was added to each well, followed by addition of 100 μl Jurkat cells (1×10⁶ cells/ml) in adhesion buffer, and incubation carried out at 37° C. for 30 min. Adherent cells were fixed by additional of 50 μl/well 14% glutaraldehyde in D-PBS and incubation at room temperature for 2 hr. The plates were washed with dH₂O and stained with 50 μl/well 0.5% crystal violet in 10% ethanol for 5 min at room temperature. The plates were washed in several changes of dH₂O, after which 70% ethanol was added. Adherent cells were quantitated by determining absorbance at 570 nm and 410 nm using a SPECTRmax 250 microplate spectrophotometer system (Molecular Devices, Sunnyvale Calif.). The percentage of cell binding was determined using the formula below.

$$\% Binding = \frac{A570 - A410 ( binding to collagen )}{A570 - A410 ( binding to mAb 3 S3 )} \times 100$$

Data was normalized using the formula:

$$\% of DMSO binding = \frac{\% of cell binding , inhibitors}{\% of cell binding , DMSO} \times 100$$

One hundred twenty-one compounds inhibited Jurkat adhesion to rat type I collagen at a level of 50% or greater than the control. IC50 determinations for these inhibitors were assessed in Jurkat adhesion assays as described above except that inhibitors were tested at two-fold dilutions through the concentration range of 0.15 to 40 μM. The IC50 values of 113 of the 121 compounds were determined and 21 of these 121 were selected based on potency in the IC50 range of 2 to 17 μM in the assay and for specificity in showing low level inhibition in the α₄β₇ binding assay (described herein) and the von Willebrand factor binding assay (described herein). These 21 compounds were further analyzed for specificity and toxicity. In specificity determinations, compounds were tested in a concentration range of 0.15 μM to 20 μM for the ability to inhibit Jurkat cell binding to immobilized VCAM-1/Ig. The assay was carried out in a manner similar to the collagen adhesion assay described above except that cells were coated with VCAM-1/Ig instead of collagen. Binding to VCAM-1 was dependent on surface expression of α₄β₁. These results are shown in Table 6.

For 21 compounds, toxicity of Jurkat cells was assessed following a four hr or 24 hr incubation. LD50 concentrations were determined using a CellTiter 96® Aqueous One Solution Cell Proliferation Assay System (Promega) according to the manufacturer's suggested protocol. A two-fold serial dilution series of each compound was tested in a concentration range of 40 μM to 0.15 μM. Results from the toxicity assay are shown in Table 6.

TABLE 6

| | α₂β₁/COLLAGEN | α₇β₁/VCAM | TOXICITY |
|---|---|---|---|
| COMPOUND | EC50 (μM) | EC50 (μM) | LD50 (μM) |
| Cmpd AD | 2 | >20 | 3 |
| Cmpd T | 4 | 15 | 25 |
| Cmpd AF | 6 | >20 | 35 |
| Cmpd AI | 6 | >20 | 33 |
| Cmpd AG | 7 | >20 | 35 |
| Cmpd AE | 7 | >20 | 30 |
| Cmpd Y | 7 | >20 | 25 |
| Cmpd J | 8 | >20 | >40 |
| Cmpd X | 8 | >20 | 30 |
| Cmpd M | 8 | >20 | 25 |
| Cmpd AL | 8 | >20 | 20 |
| Cmpd AJ | 8 | >20 | 38 |
| Cmpd AK | 9 | >20 | 35 |
| Cmpd AH | 9 | >20 | 38 |
| Cmpd AB | 13 | >20 | >40 |
| Cmpd A | 14 | 17 | >40 |
| Cmpd U | 15 | >20 | >40 |
| Cmpd G | 16 | >20 | >40 |
| Cmpd E | 16 | >20 | >40 |
| Cmpd B | 17 | >20 | >40 |
| Cmpd AN | 17 | >20 | >40 |

### EXAMPLE 12

### Identification of Alpha1 Antagonists of Collagen Binding

Chinese hamster ovary (CHO) cells do not express endogenous collagen receptors. Accordingly, CHO cells were transfected with a full-length alpha1 expression construct, alpha1/pDC-1/1. The full-length alpha1 insert was removed from a clone in the vector pLEN [Briesewitz et al., JBC 268:2989-2996 (1993)] and subcloned into the pDC-1 to generate the clone alpha1/pDC-1/1. Transfectants were grown in selective media (DMEM/F12 with 10% FBS) and cloned by limiting dilution. Alpha1 expressing clones were identified by staining the cells with a blocking alpha1 monoclonal antibody (antibody 5E8D9; Upstate Biotech) and determining expression levels by FACS analysis. These cells were demonstrated to adhere to type IV collagen in an alpha1-dependent manner using the blocking alpha1 monoclonal antibody (Upstate Biotech; clone 5E8D9) which was shown to inhibit this adhesion. In view of this result, the alpha1 transfected CHO cells were used in an adhesion assay as described below to identify inhibitors of alpha1 binding. This assay is a modification of the procedure used to identify alpha2 antagonists described above.

Immulon 4 plates were coated overnight at 4° C. with either (i) 50 μl per well human type IV collagen (Sigma) (0.5 μg/ml in CMF-PBS), (ii) the anti-alpha1 monoclonal antibody 5E8D9 in bicarbonate buffer, pH 9.6, or (iii) bicarbonate buffer alone. Plates were washed twice with D-PBS and blocked with 1% BSA (100 μl/well) in D-PBS for 1 hour at room temperature. Wells were rinsed once with 100 μl/well adhesion buffer (DMEM/F12 media with no serum). Adhesion buffer (200 μl) with or without candidate inhibitor was added to each well followed by the addition of 100 μl of alpha1 transfected CHO cells in adhesion buffer. CHO cells were previously recovered using versene and rinsed 3 times in DMEM/F12 media containing 10% FBS. Cells were resuspended in adhesion buffer at a density of 0.75×10⁶ cells/ml. Incubation of the alpha1-transfected CHO cells on type IV collagen was carried out at 37° C. for 30 minutes. Adherent cells were fixed by additional 50 μl/well 14% glutaraldehyde in D-PBS and incubation at room temperature for 2 hours. The plates were washed with dH2O and stained with 50 μl/well 0.5% crystal violet in 10% ethanol for 5 minutes at room temperature. The plates were washed in several changes of dH2O after which 70% ethanol was added. Adherent cells were quantitated by determining absorbance at 570 nm and 410 nm using a SPECTRmax 250 microplate spectrophotometer system (Molecular Devices, Sunnyvale Calif.). The percentage of cell binding was determined using the formula below.

$$\% binding = \frac{( A570 - A410 ( binding to collagen )}{( A570 - A410 ( binding to mAb 5 E8D9 )} \times 100$$

Data was normalized using the formula:

$$\% of DMSO binding = \frac{\% of cell binding , inhibitors}{\% cell binding , DMSO} \times 100$$

Sixty-four compounds inhibited alpha1-transfected CHO cell adhesion to type IV collagen by a level of 50% or greater than the DMSO-control. EC50 determinations for these compounds were determined in alpha1-transfected CHO cell adhesion assays as described above, except that the inhibitors were tested at two-fold dilutions through the concentration range of 0.15 μM to 20 μM (i.e., 0.15 μM, 0.3125 μM, 0.625 μM, 1.25 μM, 2.50 μM, 5 μM, 10 μM, 20 μM). The EC50 values for these compounds ranged from 0.5 μM to 18 μM. These compounds were further analyzed for selectivity and toxicity.

For initial specificity testing, the compounds were tested in a concentration range of 0.15 μM to 20 μM for the ability to inhibit alpha2-transfected CHO cell adhesion to type I collagen. For this assay CHO cells were transfected with an alpha2 expression construct, alpha2/pDC-1/8. The original alpha2 construct was in the expression vector pcDNA-3 and was a Genestorm clone purchased from Invitrogen. The alpha2 sequence was subcloned into pDC-1 resulting in the clone alpha2/pDC-1/8. Alpha2-expressing cells were cloned and analyzed by FACS using an alpha2 monoclonal antibody, A2-IIE10 (Upstate Biotech). A CHO cell line expressing moderate levels of alpha2 was identified and used in adhesion assays as described above for alpha1. The only differences in the alpha2 adhesion assay included (i) using immobilized rat type I collagen (Sigma) in place of the type IV collagen and (ii) using the alpha2 monoclonal antibody, A2-IIE10, in place of the alpha1 monoclonal antibody. Most compounds had a narrow range of specificity for alpha1 compared with alpha2. These compounds were about 1-3 fold more potent in inhibiting alpha1 dependent adhesion than for inhibiting alpha2 dependent adhesion.

The toxicity of the compounds was assessed in a 4 hour assay using the alpha1-transfected CHO cells. LD50 concentrations, (or “Lethal Dose 50”), as used herein, is the compound concentration necessary to kill 50% of the cells over a defined time interval. LD50 concentrations were determined using a CellTiter 96 Aqueous One Solution Cell Proliferation Assay System (Promega) according to the manufacturer's suggested protocol. A two-fold serial dilution series of each compound was tested in a concentration range of 40 μM to 0.15 μM. Toxicities for these compounds ranged from 2.5 μM to 40 μM.

Thirty-two compounds which were chosen based on their potency, selectivity and toxicity profiles were further analyzed for specificity. Compounds were tested for inhibiting adhesion of more distantly related I domain-containing integrins alphaL (LFA-1) and alphaM (Mac-1). For alphaL, the compounds were tested for inhibition of JY8 cell adhesion to ICAM-1.

ICAM-1/JY-8 Cell Adhesion Assay

Biologically relevant activity of the compounds in the present invention was confirmed using a cell-based adhesion assay that measures the ability of the compounds to block adherence of JY-8 cells (a human EBV-transformed B cell line expressing LFA-1 on its surface) to immobilized ICAM-1, as follows. Compounds were screened for the inhibition of LFA-1 dependent adhesion, as described with respect to the alpha1 assay, with some modifications. Plates were coated with ICAM-1 Ig protein (5 μg/ml in sodium bicarbonate buffer solution) instead of type IV collagen. JY cells were used in place of K562 [α₁] cells. The capture monoclonal antibody used was 22F12C (at 5 μg/ml in sodium bicarbonate buffer solution) in place of an alpha1 monoclonal antibody.

For alphaM, the compounds were tested for inhibition of Mac-1 transfected JY cell adhesion to iC3b (assay described in Example 2). For both assays, compounds were tested in a 2-fold dilution series in a concentration range from 20 μM to 0.15 μM. Most compounds were 1-10 fold more effective at inhibiting alpha1 dependent adhesion than inhibiting LFA-1 and MAC-1 dependent adhesion. These compounds were also analyzed for toxicity in a 4 hour assay with the JY cells as described above for the CHO cells.

A second alpha1-dependent cell adhesion assay was developed to further assess the alpha1 antagonists identified. K562 cells, a myeloid leukemia cell line, was transfected with a full-length alpha1 expression construct alpha1/pMHneo/40. The alpha1/pMHneo/40 construct was generated by subcloning the full length alpha1 sequence into the expression vector pMH-neo [Hahn et al., Gene 127:267-268 (1993)]. Transfectants were selected with 0.5 mg/ml G418. In order to further select for alpha1-expressing cells, the transfectants were panned for adhesion to type IV collagen. For the panning, tissue culture plates were coated with 20 mg/ml of human type IV collagen (Sigma) in CMF-PBS for 1 hour at 37° C. The plates were washed with binding buffer (RPMI with 10% FBS) and the alpha1-transfected K562 cells were added in binding buffer containing 20 ng/ml PMA. After incubation at 37° C. for 1 hour, the plates were washed to remove unbound cells. Adherent cells were removed with versene and diluted with binding buffer. After panning, K562 cell lines expressing alpha1 were obtained and used for further screening described below.

Twenty-one alpha1 antagonists identified in the CHO cell adhesion assay were further analyzed in an alpha1-transfected K562 cell adhesion assay. The cell adhesion assay was performed as described above for the CHO cell assay except that RPMI was used as the adhesion buffer. The potencies of the alpha1 antagonists were similar in the K562 and CHO cell adhesion assays with most EC50 values for most compounds falling within a 1-3 fold range between the two assays. The compounds were also analyzed for toxicity with the K562 cells in a 4 hour assay using the CellTiter 96 Aqueous One Solution Cell Proliferation Assay System described above. The toxicities (LD50) of most compounds was similar in the K562 and the CHO assays. The LD50 values between the two assays varied by less than 2-fold for the majority of compounds.

The structures of five alpha1 antagonists are shown in Table 7. These compounds have EC50 values in the range of 0.5-1.5 μM. These compounds have narrow specificity for alpha1 over alpha2 (1-4 fold), and greater selectivity over more distantly related integrins, such as LFA-1 and Mac-1 (3-10 fold). The window between potency (EC50) and toxicity (LD50) ranges from 6-20 fold.

TABLE 7

| | |
|---|---|
| AT | 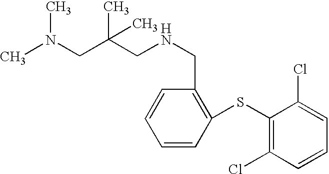 |
| AU | 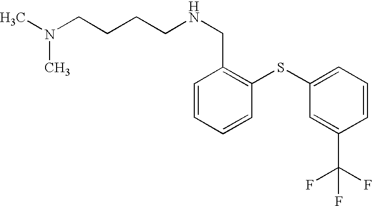 |
| AV | 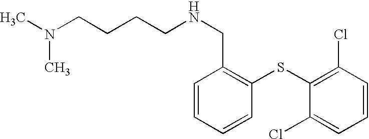 |
| AW | 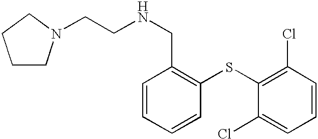 |
| AX | 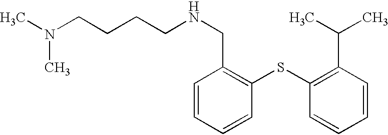 |

### EXAMPLE 13

### Expression and Purification of Alpha1 I Domain and its Usage in a Biochemical Assay

An alpha1 I domain construct was generated for expressing the alpha1 I domain as a histidine tagged protein in E. coli. The histidine-tagged protein was used in co-crystallization experiments to determine the 3-dimensional structure of the alpha1 I domain complexed with inhibitors. The histidine tagged protein was also used to assess alpha1 antagonists in a biochemical assay by measuring the binding of the alpha1 I domain to immobilized collagen.

The alpha1 I domain was cloned as follows. A polynucleotide encoding the alpha1 I domain was PCR amplified using the A1.I.Bam (SEQ ID NO: 26) and A1.I.Pst (SEQ ID NO: 27) primers shown below and the vector alpha1/pDC-1/1 as template.

| | |
|---|---|
| A1.I.Bam: | |
| CGGATCCCCCACATTTCAAGTCGTGAAT | SEQ ID NO:26 |
| A1.L.Pst: | |
| GCTGCAGTCATATTCTTTCTCCCAGAGTTTT | SEQ ID NO:27 |

PCR conditions included an initial incubation at 94° C. for 2 minutes, followed by 30 cycles of 94° C. for 30 seconds; 55° C. for 30 seconds and 72° C. for 30 seconds; and a final incubation of 72° C. for 7 minutes. The resulting PCR product was gel purified, digested with BamHI and PstI, gel purified again and then cloned into the vector pQE30 (Qiagen) previously digested with BamHI and PstI. The resulting clone alpha1/pQE30/2 was verified by sequencing.

The alpha1/pQE30/2 construct was transformed into E. coli strain M15(pRFP4) (Qiagen) for protein expression. Histidine-tagged alpha1 I domain was solubilized from purified inclusion bodies using 6 M guanidine and then snap refolded by dilution in buffer without guanidine. The solubilized alpha1 I domain was purified using a Ni-NTA agarose column (Qiagen) and elution with an imidazole gradient.

The purified alpha1 I domain was used in direct binding assays with immobilized type IV collagen as follows. Costar Immulon4 plates (96 well) were coated overnight with either (i) 50 μl/well of human collagen IV protein (Sigma) at 40 μg/ml in CMF-PBS, (ii) anti-alpha 1 I-domain monoclonal antibody (Immune Diagnostics) at 10 μg/ml in CMF-PBS (positive control), or (iii) CMF-PBS alone (negative control). Plates were incubated overnight at 4° C. The next day, media was removed and the plates were blotted dry, after which 150 μl/well of 2% BSA in CMF-PBS containing 0.05% Tween-20 was added to block the plates, and plates were incubated further at 37° C. for 1 hour. Media was again removed from the plates which were blotted dry, and then washed twice with 150 μl/well of CMF-PBS containing 0.05% Tween-20 and 5 mM MgCl₂ (PBS/T/Mg). Approximately 50 μl/well of PBS/T/Mg containing 2×compound, DMSO, anti-alpha 1 I-domain monoclonal antibody, isotype-matched control monoclonal antibody or no inhibitor was added to the plates, after which 50 μl/well of PBS/T/Mg containing 20 μg/ml of purified alpha 1 I-domain was added and plates were incubated for 30 minutes at 37° C. Media was removed and the plates were blotted dry, then washed twice with 100 μl/well PBS/T/Mg, after which 100 μl/well PBS/T/Mg containing 1 μg/ml anti-penta-His monoclonal antibody (Qiagen) was added. The plates were then incubated for 30 minutes at 37° C., the media was removed, and the plates blotted dry. The plates were then washed twice with 100 μl/well PBS/T/Mg, 100 μl/well PBS/T/Mg containing a 1:20,000 dilution of GAM-HRP (Sigma) was added, and the plates were incubated for 30 minutes at 37° C. Media was removed, and the plates were blotted dry. The plates were washed twice with 100 μl/well PBS/T/Mg and 100 μl/well of Substrate Buffer containing 150 μl/l of H₂O₂ and a 1:100 dilution of TMB substrate stock was added to each well and the plates were developed in the dark for 30 minutes at room temperature. Fifty μl/well of 15% H₂SO₄ was then added to stop the reaction and the plates were analyzed by A₄₅₀-A₆₇₀ on a spectrophotometer. The specific signal was determined by subtracting background binding to “negative control” wells.

An additional assay was developed using Europium-labeled alpha 1 I domain and bound I domain was directly detected after washing using time resolved fluorescence (TRF). In this assay, purified alpha-1 I domain was labeled with Europium using a DELFIA Europium-labeling kit according to the manufacturer's suggested protocol (Wallac). Costar Immulon4 plates (96 well) were coated with 100 μl of 25 μg/ml of human collagen IV protein (Sigma) in CMF-PBS/1 μM MgCl₂, and incubated overnight at 4° C. Plates were then washed 3 times with TBS/T (20 mM Tris, pH 8.0; 150 mM NaCl; 0.02% Tween-20) and 1 mM MgCl₂ (TBS/T/Mg), 200 μl per well. Plates were then blocked with CMF-PBS/1 mM MgCl₂/2% BSA, 100 μl per well for 1 hour at 37° C. Plates were washed again, then probed with 5 g/ml Europium I domain in RPMI/5% TBS/1 mM MgCl₂, 100 μl per well, and incubated at 37° C. for one hour. Plates were then washed again and developed by adding 100 μl/well Enhance (Wallac) and analyzed on a Victor plate reader by time resolved fluorescence.

### EXAMPLE 14

### Expression and Purification of Alpha1Beta1 Leucine Zipper Protein and its Usage in a Biochemical Assay

In order to develop a more physiologically accurate biochemical assay, an alpha1 beta1 leucine zipper protein was generated. Expression constructs were prepared individually encoding the full length extracellular domains of alpha1 and beta1 without the transmembrane and cytoplasmic tail polypeptide sequences. Removal of the transmembrane regions allows these proteins to be secreted from transfected cells providing easy purification. The transmembrane sequences were replaced with the acidic and basic leucine zipper sequences respectively. See generally, Chang et al., PNAS 91:11408-11412 (1994).

The extracellular domain of alpha1 was subcloned from the original alpha1 clone in pLEN [Briesewitz et al., JBC 268:2989-2996 (1993)]. The extracellular domain of beta1 were subcloned from the full length beta1 clone He6.1.2/pcDNA-1 Amp. This clone was obtained by screening a Hela cDNA library by hybridization. The leucine zipper sequences promote the formation of the alpha1beta1 heterodimer. These constructs were generated using the same leucine zipper sequences and vectors described in U.S. Pat. No. 6,251,395, issued on Jun. 26, 2001, Example 14 of which is hereby incorporated herein by reference for its description of methods for constructing leucine zipper proteins. The alpha1 and beta1 leucine zipper constructs were co-transfected into CHO cells which were then maintained in DMEM/F12 media with 10% dialyzed FBS. Supernatant was collected and the secreted alpha1beta1 heterodimer was purified using chromatography over CNBr-activated Sepharose 4B (Pharmacia) coupled with an anti-leucine zipper monolconal antibody which recognizes both chains of the leucine zipper. For use in biochemical assays, purified alpha1beta1 leucine zipper protein was Europium labeled using a DELFIA Europium-labeling kit according to the manufacturer's suggested protocol. Binding of the labeled alpha1beta1 leucine zipper protein to immobilized collagen was measured by time resolved fluorescence. The heterodimer assay was set up essentially the same as the Europium labeled I domain assay, with the exception that Europium labeled heterodimer in CMF-PBS/1 mM MgCl₂/2% BSA was substituted as the probe for the Europium labeled I domain.

### EXAMPLE 15

### Von Willebrand Factor/gpIb-CHO Static Cell Adhesion Assay

The A11 domain in von Willebrand factor (vWf) is homologous to I domains found in other proteins. To investigate the possibility that these molecules might be susceptible to similar modulation as described above, the library of test compounds were tested for the ability to modulate vWf binding to gpIb.

Round-bottom (RB) glass plates were coated overnight at 4° C. with 50 μl of 1 μg/ml bovine vWf (bvWf) in CMF-PBS. Control wells include wells that were coated with 5 μg/ml of vWf at 50 μl/well, or with fibrinogen at 10 μg/ml. The next morning, the plate was washed once with 200 μl of CMF-PBS, blocked with 200 μl of 2.5% gelatin for 1 hr at 37° C., and washed three times with 200 μl CMF-PBS.

CHO cells transfected with DNA encoding glycoprotein(GP) Ib-IX [Cranmer, J. Biol. Chem. 274:6097-6106(1999)] were grown in DMEM/F12 with 10% FCS, antibiotics, glutamine and 5-hydroxytryptophan supplemented with 400 μg/ml G418 and 200 μg/ml zeocin (Invitrogen). Confluent cells were washed once with CMF-PBS and incubated with warm Versene in incubator for 5 min. Cells were collected and resuspended in Tyrode's solution (Sigma) with 4 mM EDTA at a density of 2×10⁶ cells/ml.

The library of test compounds were diluted in Tyrode's/EDTA to 20 μM and 50 μl of the diluted compound was added to each well to a final concentration of 10 μM. For the control, 1 μl 100% DMSO was added to 300 μl cell, with the final concentration of DMSO 0.3%. In a control with a known vWf inhibitor, aurin-tricarboxylic acid (ATA, Sigma) was dissolved in 100% DMSO to 20 mM, diluted with Tyrode's/EDTA to 20 μM, and 50 μl/well to final concentration of 10 μM was added.

Cells were added to each well at a density of 10⁵ cells/well in 50 μl and the plates rocked for 40 min at room temperature. The non-adherent cells were removed by aspiration, 200 μl CMF-PBS was added, the plates vortexed and the buffer removed. Calcein was added (50 μl/well of a 2 μM stock) and the plates incubated at room temperature for 1 hr to label adherent cells. Fluorescence was measured on a Millipore CytoFluor 2350 fluorimeter to quantitate adherent cells. A number of compounds having IC50 values less than 20 μM were identified.

### EXAMPLE 16

### CD11b-Mediated Neutrophil Adhesion to Fibrinogen

The adhesion assay described above for CD18/CD11b-(Mac-1)-mediated adhesion of HL-60 cells to ICAM-1 was carried out with the following modifications. Each well was coated overnight at 4° C. with 50 μl of glycophorin (10 μg/ml), fibrinogen (5 μg/ml) or with anti-CD18 monoclonal antibody (22F12C, 5 μg/ml) and anti-CD11b monoclonal antibody (44AACB, 5 μg/ml) in 50 mM bicarbonate buffer (pH 9.6). Plates were blocked with 1% human serum albumin and no blocking antibody was used. Neutrophils were isolated from fresh heparin whole human blood by density gradient centrifugation and 100 μl of the cells (4×10⁶ cells/ml) in adhesion buffer was added to each well. Plates were incubated at 37° C. for 10 minutes.

Over 1000 compounds were screened, and several had IC50 values ranging from 1 μM to 40 μM. Nine compounds were found to have IC50 values below 10 μM [Cmpd S, Cmpd R, Cmpd N, Cmpd O, Cmpd P, Cmpd Q, Cmpd L, Cmpd V, and Cmpd F, as set out in Table 2. After SAR efforts based on compound Cmpd S (which initially showed an IC50 of 1 μM), inhibition potency for compounds improved to less than 200 nM (for Cmpd AA and Cmpd AC) and several compounds showed complete inhibition at 20 μM [compounds ranging from Cmpd Z to Cmpd AM]. With the exception of Cmpd Z all compounds selected for their ability to inhibit α_{E}β₇/E-cadherin also antagonize the other β₇ integrin, α₄β₇. These compounds also exhibited minimal inhibitory activity towards α_{L}β₂, α_{d}β₂, and α₄β₁. Also, relative to α_{E}β₇/E-cadherin, these compounds display limited selectivity (less than 2-fold) for α_{M}β₂ and α_{V}β₃.

### EXAMPLE 17

### Development of Inhibitors of Rac1 Guanine Nucleotide Exchange Reaction

Rac proteins are not active when bound to GDP, but are activated by the exchange of GDP for GTP. The exchange of GDP for GTP in Rac proteins is catalyzed by guanine nucleotide exchange factors (GEFS) such as, Vav1 and Tiam1 [Aghazdeh et al., Cell 102:625 (2000); Worthylake et al., Nature 408:682 (2000)]. Due to the importance of Rac proteins in the control of cell proliferation, antagonists of the Rac guanine nucleotide exchange reaction and, in particular, small molecules that interfere with the exchange of GDP for GTP of Rac1 in the presence of Tiam1, are of considerable interest for the methods and compositions of the present invention.

A. Cloning and Expression of Rac1 and Tiam1:

Rac1 and the DH-PH domain of Tiam1 were cloned using standard recombinant DNA procedures [Disbury et al., J. Biol. Chem. 264:16378 (1989)]. Rac1 was expressed in E. coli as a GST fusion protein using the vector pGEX2T in accordance with previously described methods [Self and Hall, Meth. Enzymol. 256:3 (1995)]. Purified thrombin-cleaved Rac1 protein was used in the assay.

The Tiam1 DH-PH domain expressed as a fusion protein containing a carboxy terminal 6XHis tag using the plasmid pET28a described by Rossman and Campbell, Meth. Enzymol. 325:25 (2000).

B. Guanine Nucleotide Exchange Assay:

The Tiam1-catalyzed exchange of GDP for GTP of Rac1 was carried out essentially according to the procedure described by Crompton et al., J. Biol.Chem. 275(33):25751 (2000). GDP-bound Rac1 was incubated with [α³²P]-labeled GTP, in the presence of Tiam1 and nucleotide exchange was monitored by following the increase in radioactivity bound to Rac1. Free radioactivity was removed by placing the reaction mixture in the well of a 96-well plate and filtering out the fraction of [α³²P]GTP that is not bound to Rac1. During the screen for the nucleotide exchange antagonists, the compounds were used at 10 μM concentration. The compounds analyzed by the screening methods are further described below.

C. Cell Proliferation Assay:

Rat embryonic fibroblast (REF) and Jurkat cells were selected as representatives of fibroblastic and T cells respectively in order to test the effect of Rac1 guanine nucleotide exchange inhibitors on cell proliferation. REF cultured in the medium RPMI (“REF-R cell culture”) was obtained as described by Nobes, Meth. Enzymol. 325:441 (2000). REF-R or Jurkat cells in complete RPMI and 10% fetal bovine serum (FBS) were plated into 96 well plates in duplicate, 10×10³ cells/well. After 21 hrs (for Jurkat), and 45 hrs (for REF-R), AlamarBlue (Serotec) was added, and cells were returned to the incubator (37° C., 5% CO₂) for additional 3 hrs. Results were obtained with SpectraMaxGEMINI (Molecular Devices).

Compounds that inhibit the Rac1 guanine nucleotide exchange reaction by at least 50% of the control were obtained. The IC50 value of several of the compounds were determined for the guanine nucleotide exchange reaction of Rac1, in the presence of Tiam1. The five structures shown in Table 8 represent the most potent inhibitors, and IC50 values for the guanine nucleotide exchange reaction for these compounds are also included therein.

TABLE 8

| Compound | | IC50 (μM) |
|---|---|---|
| AY | 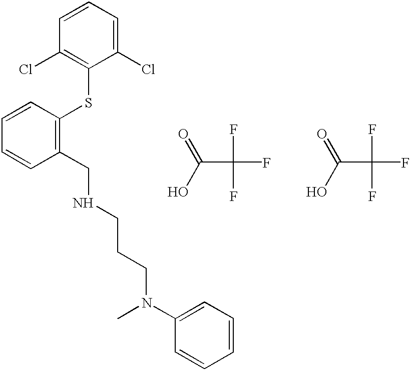 | 1.9 |
| AZ | 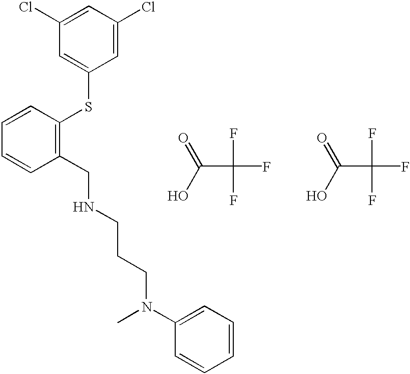 | 3.1 |
| AAA | 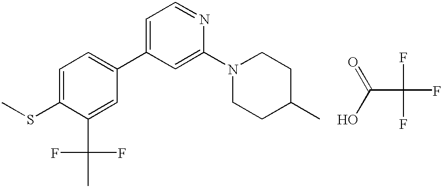 | 3.5 |
| AAB | 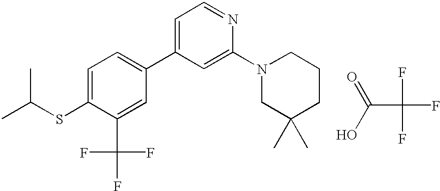 | 4.7 |
| AAC | 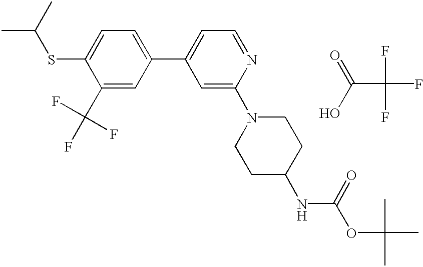 | 5 |

### EXAMPLE 18

### Inhibition of Bacterial Proteins

Three microbial enzymes containing Rossmann fold structures were identified as candidates for screening with the library of test compounds. Selection was based on (i) presence of the Rossmann structure; (ii) expression patterns in prokaryotic and eukaryotic cells; (iii) clinical importance; and (iv) functional importance to bacterial growth and survival. Two of the selected proteins, dihydrodipicolinate reductase (DHPR or DapB) and enoyl-acp reductase (ENR), catalyze electron transfer from NADH to a substrate and are integral to biosynthetic pathways for lysine synthesis and fatty acid synthesis, respectively. The third and fourth proteins, E. coli ras-like GTPase (ERA-GTAse) and yihA (also a GTPase), are involved in translation and cell cycle regulation.

Modulation of DapB activity is assessed using an optical assay that involves synthesis of dihydrodipicolate from aspartate semialdehyde. The assay utilizes dihydrodipicolate synthease (DapA) to first synthesize dihydrodipicolinate, followed by addition of NADH and DapB. A coupled reaction is necessary because dihydrodipicolate is an unstable compound. The change in absorbance in the presence and absence of a test compound resulting from NADH conversion to NAD⁺ is monitored at 340 nm.

Identification of modulators of ENR is carried out in a similar manner, but in a single step reaction. Briefly, NADH and ENR are first incubated, followed by addition of substrate, (either crotonyl-CoA or crotonyl-ACP). Again, the change in absorbance in the presence and absence of a test compound resulting from NADH conversion to NAD⁺ is monitored at 340 nm.

In view of the fact that optical assays require large amount of substrate, i.e., crotonyl CoA, and the fact that several test compounds absorb at the same wavelength as NADH, alternative thin layer chromatography (TLC) and plate-based assays were designed to identify modulators of ENR using radiolabeled NADH.

The TCL method measures conversion of ³²P-NADH to NAD⁺ in the presence of lithium chloride which causes the two sates to separate on PEI membranes after a 5 to 10 min run time. Radiolabeled spots are measured on a Storm Phosphoimager and the ratio of NAD⁺ to NADH is calculated. An increase in the ratio of NADH to NAD⁺ in the presence of a test compound is indicative of inhibition of the conversion. The control reaction is optimized to measure conversion in the linear range. Practical application of this assay was demonstrated using a commercially available enzyme inhibitor. The TLC method is particularly useful for small scale screening.

For large scale screening, the plate based assay is designed to utilize the same reagents. This assay exploits the charge difference between NADH and NAD⁺ to permit separation. Positively-charged DEAE-cellulose membrane is used to selectively trap ³²P-NADH which has a net negative charge greater than NAD⁺. Trapped NADH is detected using scintillation counting and increased signal in the presence of a test compound indicates enzyme inhibition.

For ERA-GTPase, a one step assay is carried out to identify modulators. The transfer of labeled phosphorus in the conversion of GTP to GDP is measured in the presence and absence of a test compound, the label being detected in a scintillation counter using an assay routinely practiced in the art. Conditions for the ERA GTPase assay can also be utilized in screening for yihA modulators.

### EXAMPLE 19

### Identification of HPPK Antagonists

The enzyme 6-hydroxymethyl-7,8-dihydropterin pyrophosphokinase (HPPK) is part of the de novo folate biosynthetic cascade and catalyzes the transfer of pyrophosphate from ATP to 6-hydroxy-7,8-dihydropterin (HMDP) [Richey et al., J. Biol. Chem. 244:1582-1592 (1969)]. HPPK is expressed in both gram positive and gram negative bacteria, fungi, and protozoa, but not in higher eukaryotes. Accordingly, HPPK represents a novel target for the development of antibiotics with anti-folate activity.

1. Isolation of the E. coli HPPK Gene

The E. coli HPPK gene was isolated by PCR amplification of E. coli genomic DNA with the following oligonucleotide primers specific for the 5′ (SEQ ID NO: 28) and 3′ (SEQ ID NO: 29) ends of the HPPK gene:

| | | |
|---|---|---|
| 5′EcHPPK | 5′-GTAGATGACAGTGGCGTATATT-3′ | SEQ ID NO:28 |
| 3′EcHPPK | 5′-GCCTTACCATTTGTTTAATTTGT-3′ | SEQ ID NO:29 |

PCR was performed in a Perkin Elmer Cetus (PE Applied Biosystems, Foster City, Calif.) DNA thermal cycler under standard conditions. See generally, Ausubel et. al, Current Protocols in Molecular Biology, Vol. 3, p. 15.1.1-p.15.1.15 (1999). The amplification products were then analyzed by agarose gel electrophoresis to determine the approximate size of the PCR product, and a single DNA fragment of approximately 487 bp was detected, as anticipated.

The HPPK PCR product was ligated to the vector pCRII-TOPO (Invitrogen Corp., Carlsbad, Calif.) according to the manufacturer's protocols. E. coli strain TOP10 (Invitrogen Corp., Carlsbad, Calif.) was transformed with an aliquot of the ligation reaction, as recommended by the manufacturer, and single bacterial colonies were isolated and grown overnight in LBM media containing 100 μg/ml carbenicillin.

Plasmid DNA was isolated from 2 ml cultures of the single colonies using the Wizard Plus Miniprep Kit (Promega Corp., Madison, Wis.). The DNA sequence of the E. coli HPPK PCR product in the plasmid pCRII-TOPO/EcHPPK was determined to be correct, having the amino acid sequence set out in SEQ ID NO: 30.

2. Generation of His(6)-HPPK Expression Constructs

In order to facilitate the purification and detection of E. coli HPPK, the following changes were made to the E. coli HPPK coding sequence during a subsequent PCR amplification: 1.) the amino terminus of HPPK was modified to incorporate an additional 6 histidine residues, and 2.) unique restriction sites were added to the 5′ and 3′ ends of the coding region to facilitate subcloning of the PCR fragment into the expression vector pBAR5. Methods for the subcloning of a similar PCR fragment into an expression vector have been previously described in U.S. Pat. No. 5,847,088, issued Dec. 8, 1998, Example 8 of which is hereby incorporated herein by reference. The 5′ PCR primer included an NcoI restriction site followed by sequences encoding the additional amino acid residues “MGHHHHHHGG” (SEQ ID NO. 31) as shown below:

| | |
|---|---|
| 5′EcHisHPPK | |
| 5′-CGCCATGGGCCACCACCACCACCACCACGGCGG | SEQ ID NO:32 |
| CATGACAGTGGCGTATATT-3′ | |

The 3′ PCR primer included a XhoI restriction site and is shown below:

| | |
|---|---|
| 3′EcXhoHPPK | |
| 5′-CGGCTCGAGTTACCATTTGTTTAATTTGT-3′ | SEQ ID NO:33 |

Using these primers, the 487 bp HPPK PCR product was amplified in a standard PCR amplification reaction, and an aliquot of the reaction was analyzed by agarose gel electrophoresis. A single band of approximately 519 bp that corresponded to the anticipated size was detected. The PCR amplification product was purified using a QIAquick PCR Purification Kit (QIAGEN Inc., Valencia, Calif.), and digested with the restriction enzymes NcoI and XhoI. The digested PCR product was ligated into NcoI- and XhoI-digested plasmid pBAR5, and an aliquot of the ligation reaction was used to transform TOP10 bacteria according to the manufacturer's protocols (Invitrogen Corp., Carlsbad, Calif.). Single colonies were isolated after plating on LBM agar plates containing carbenicillin. Several of the single colonies were grown overnight in 2 ml cultures of LBM containing carbenicillin, and plasmid DNA was isolated for DNA sequencing as previously described. The plasmid pBAR5/HisHPPK was shown to contain an open reading frame encoding the His(6)-HPPK gene having the following amino acid sequence set out in SEQ ID NO: 34.

3. HisHPPK Expression

Plasmid pBAR5/HisHPPK was used to transform the E. coli strain BL21(DE3)pLysS (Novagen Inc., Madison, Wis.) using standard methods. Transformants were selected after plating onto LBM plates containing both chloramphenicol and carbenicillin, to select for the plasmids pLysS and pBAR5/HisHPPK respectively. Plasmid pLys is a plasmid that encodes T7 lysozome. The presence of lysozome aids cell lysis following a freeze-thaw cycle.

To initiate large-scale expression of HisHPPK, a 50 ml culture of BL21(DE3)pLysS containing pBAR5/HisHPPK was grown overnight at 30° C. with shaking in LBM containing carbenicillin and chloramphenicol. The following day, 10 ml of the overnight culture was used to inoculate 2 liter flasks containing 500 ml of LBM supplemented with carbenicillin and chloramphenicol. The flasks were incubated at 37° C. with shaking until the bacterial cultures reached an OD₆₀₀ of approximately 0.6.

The plasmid pBAR5/HisHPPK contains an arabinose-inducible promoter upstream of the HisHPPK gene. Once the cultures reached appropriate density, arabinose was added to the cultures to a final concentration of 0.1% to induce HisHPPK expression, and the flasks were incubated at 37° C. with shaking for another 2.5 hours. The bacteria were then harvested by centrifugation and the cell pellet from 1 liter of bacterial culture was resuspended in lysis buffer [50 mM Na₂HPO₄, pH 8, 50 mM imidazole, 10 mM β-mercaptoethanol, 0.5 M NaCl, and EDTA-free protease inhibitor cocktail tablets (Roche Molecular Biochemicals, Indianapolis, Ind.)] to a final volume of 35 ml. Each 35 ml bacterial suspension was transferred to a 50 ml polypropylene tube, snap frozen on dry ice, and then stored at −20° C.

4. Purification of HisHPPK

Each 35 ml aliquot was thawed on ice and lysed in a French press. To obtain a cleared lysate representing the soluble protein fraction, the lysate was centrifuged for 30 minutes at 20,000×g, at 4° C. HisHPPK was purified using a two-step procedure.

First, bacterial proteins that bound the Ni-NTA agarose (QIAGEN Inc., Valencia, Calif.) nonspecifically were removed by incubating the cleared lysate with NTA agarose which had been previously treated with EDTA to remove associated Ni²⁺ cations. The 35 ml of cleared lysate was incubated batchwise with 1 ml of NTA agarose for approximately 1 hour at 4° C. after which the NTA resin was removed by centrifugation. HisHPPK was purified on Ni-NTA agarose according to the manufacturer's protocols (QIAGEN Inc., Valencia, Calif.). The isolated HisHPPK protein was resolved on a 12% Novex gel (Invitrogen Corp., Carlsbad, Calif.), the gel was fixed and stained with Coomassie brilliant blue under standard conditions, and the only protein identified in the HisHPPK preparation was a single species of about 19 kD in mass, which corresponds to the anticipated size of HisHPPK. The protein was dialyzed against 20 mM Tris, pH 8, aliquotted, and stored at −70° C.

5. Screening Assay for HPPK Activity

In order to identify small molecule inhibitors of HPPK, an assay for HPPK measuring the HPPK-dependent conversion of ATP to AMP as a by-product of the pyrophosphorylation of 6-hydroxymethyl-7,8-dihydropterin (HMDP) was employed [Shi et al. J. Med. Chem. 44:1364-1371 (2001)]. Elevated concentrations of both substrates (HMDP and ATP) were used in the assay to reduce the possibility of identifying substrate competitors. This reaction was modified for use in 96-well V-bottom polypropylene plates as follows.

A master mix of the following composition was prepared containing 50 mM Hepes, pH 8.5, 100 μM HMDP (Schircks Laboratories, Jona, Switzerland), 10 mM MgCl₂, 35 μM adenosine triphosphate, and 10 ng of γ-labeled ³²P-ATP (Amersham Pharmacia Biotech, Arlington Heights, Ill.). An aliquot of the master mix was added to each well of the 96-well assay plate. Also added to the assay plate was 5 μl/well of the candidate inhibitor compound at a final screening concentration of 20 μM. Each candidate compound was diluted in DMSO prior to addition to the assay plate; and the final concentration of DMSO in the final assay mixture was 5%. The reaction was initiated by the addition of 100 ng of purified HisHPPK, and allowed to proceed for 15 minutes at 37° C. The reaction was stopped by the addition of an equal volume of 120 mM EDTA to each well. To resolve radiolabeled ATP from AMP, 2 μl of the reaction volume was spotted onto a PEI cellulose plate and the plate was developed with 0.3 M KH₂PO₄. The radioactivity of the plate was measured with a system Molecular Dynamics Storm 860 Phosphor imager system (Molecular Dynamics Storm, Sunnyvale, Calif.). The HPPK enzymatic activity in the presence of compound was inferred from the percent conversion of radiolabeled ATP to AMP in duplicate test samples relative to a DMSO-only control reaction. Since nonspecific background in samples lacking substrate was less that 1%, and no correction was made. Approximately 2,520 compounds were screened, and approximately 58 compounds inhibited HPPK activity by 55% or greater, yielding a hit rate of 2.3%. These compounds were ranked for in vitro potency by IC50 determinations.

6. Effect of HPPK Antagonists on the Bacterial Growth of E. coli TolC

The minimal inhibitory concentration (MIC) required to inhibit the growth of E. coli, using a microtiter broth assay, was measured in order to determine the in vivo activity of the HPPK hits. The MIC is defined as the minimum concentration required to reduce growth 80% compared to DMSO-only controls. More specifically, the efficacy of these compounds was measured against an E. coli strain containing a mutation in the TolC gene. The TolC gene encodes a transperiplasmic efflux pump which facilitates the export of small molecules such as protein toxins and antibiotics from the bacterial cytosol [Andersen et al. Curr. Opin. Cell. Biol. 13:412-416 (2001).] Although, this mutation has no affect on the entry of compounds into the bacterium, some compounds prone to elimination via the efflux pumps may reach a higher intracellular concentration in the TolC mutant. All microtiter broth assays followed those protocols established by the National Committee for Clinical Laboratory Standards [Methods for Dilution Antimicrobial Susceptibility Tests for Bacteria that Grow Aerobically; approved standard-5th Edition. Vol. 20, No.2. NCCLS Guidelines. Wayne, Pa. (2000).]

Microtiter broth assays were performed in Mueller-Hinton broth, which contains low thymidine levels. The presence of thymidine in bacterial media antagonizes the activity of the anti-folates trimethoprim and sulfamethoxazole, and likely antagonizes HPPK inhibitors as well.

Compounds were serially diluted two-fold in DMSO prior to addition to the microdilution plates. Each plate contained two controls: a serial dilution of trimethoprim provided a positive control for each plate, and a second row containing uninoculated Mueller-Hindon broth served as a sterility control for monitoring cross-contamination between wells. The inoculum density was approximately 10⁵ bacteria/ml in a final volume of 100 μl. Plates were incubated for 16 hours before OD₆₀₀ was measured.

The four compounds with the greatest activity in the MIC assays are shown in Table 9. The minimal inhibitory concentration of these compounds in E. coli TolC ranged from 0.1-12.5 μM. However, the MIC assays do not distinguish between bacteriostatic and bacteriocidal modes of action, nor do they determine if these compounds selectively inhibit HPPK in vivo. Experiments are underway to determine if these compounds have anti-folate activity and inhibit HPPK in vivo. It is well established that the activity of conventional anti-folates such trimethoprim and sulfamethoxazole are antagonized by the presence of thymidine in the bacterial medium [Amyes and Smith, J. Med. Microbiol. 7(2):143-153 (1973)]. Experiments to determine the MIC for each compound in Mueller-Hinton media alone, or following supplementation of the media with thymidine will be conducted. If the diarylsulfide compounds inhibit HPPK in vivo, then their activity should be attenuated in the presence of the folate end-product thymidine. Alternatively, these compounds can be analyzed for their ability to synergistically inhibit bacterial growth when paired with trimethoprim. Synergism would only occur if both the diarylsulfide compound and trimethoprim were acting on the same biochemical pathway. The combinatorial analysis of trimethoprim and a test compound are performed in a standard “checkerboard” study where these compounds are cross-titrated and analyzed for their effect on bacterial growth in a microtiter broth assay as previously described [Eliopoulos and Moellering, Jr., Antimicrobial Combinations, pp. 330-393, in Antibiotics in Laboratory Medicine, 4th Edition.(V. Lorian ed., 1996)].

TABLE 9

| | |
|---|---|
| AAD | 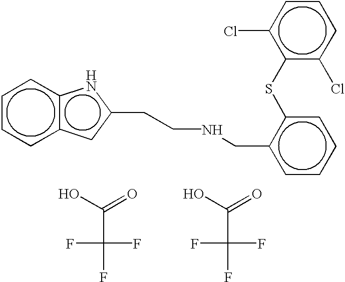 |
| AAE | 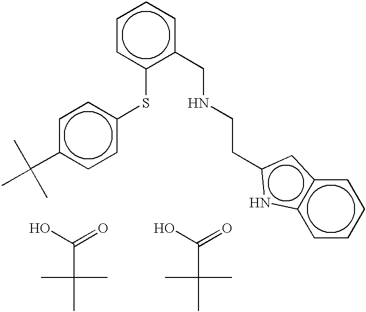 |
| AAF | 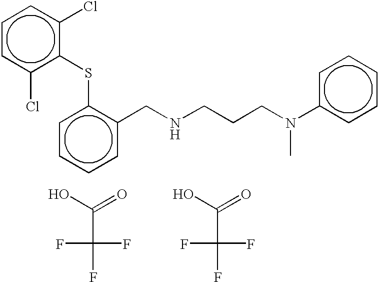 |
| AAG | 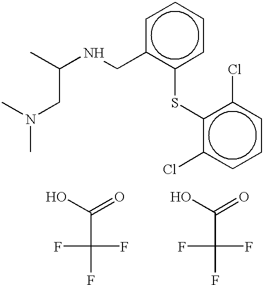 |

### Example 20

### Assays for the Identification of ftsZ Inhibitors

FtsZ is the product of an essential bacterial gene that is involved in cell division. FtsZ binds and hydrolyzes GTP, and when bound to GTP it forms long, linear polymers. The GTP-dependent polymerization of ftsZ is related to its function in bacterial cell division. During septation, ftsZ forms a ring to define the plane of cell division. Cells lacking ftsZ can not undergo septation, do not divide and die. FtsZ is highly conserved (approximately 60%) throughout the bacterial kingdom. Accordingly, ftsZ inhibitors could represent broad-spectrum antibiotics with a novel mechanism of action. The atomic structure of ftsZ, as determined by x-ray diffraction, shows that it is an alpha/beta protein [Nogales et al., (1998) Nature Structural Biology 5:451-458]. The most similar structural relative to ftsZ is the eukaryotic protein tubulin, which is a GTP-binding and hydrolyzing protein that also polymerizes to form microtubules with an essential role in the segregation of organelles and chromosomes during cell division.

A polymerization assay for the identification of ftsZ inhibitors that can be performed in microtiter wells has been devised. The polymerization assay is an adaptation of a tubulin polymerization assay [Bollag et al., Cancer Research 55:2325-2333 (1995)], and involves the reversible polymerization of ftsz in a GTP-dependent fashion.

In the presence of GTP and 10 mM CaCl₂, 5 nm ftsZ linear polymers assemble into higher order polymers [Yu et al., EMBO 16:5455-5463 (1997)] that are large enough to be trapped by a 0.2 μm filter. The protein that is retained on the filter can be stained and detected in a colorimetric assay. A reaction consisting of 300 μg/ml of ftsZ polymerized by 100 μM GTP was screened against candidate ftsZ inhibitors at 10 μM.

An alternative assay that may be more sensitive was also devised. In this assay, 100 μg/ml ftsZ was incubated with 0.5 μM ³²P-γ-GTP. The GTPase activity of ftsZ liberates ³²PO₄. By terminating the reaction with 25 mg/ml activated charcoal in 100 mM NaH₂PO₄ and centrifuging the product, the remaining ³²P-γ-GTP is trapped by the charcoal. Accordingly, the ³²PO₄ that remains in the supernatant can be measured, providing a measurement of GTPase inhibition. This screening assay may better identify ftsZ inhibitors because it is significantly more sensitive to inhibition by GDP than the polymerization screen described above (IC50 of 8 μM vs. 250 μM).

### EXAMPLE 21

### Screening Assay for ENR Inhibitors

An assay to screen for ENR inhibitors, using non-radioactive high purity NADH, was developed. The isolation of ENR is described by Baldock et al., Science 274:2107 (1996). Briefly, ENR catalyzes the conversion of NADH and crotonyl-CoA to form NAD+ and fatty acyl-CoA, and the assay measures the amount of NAD+ produced in a second reaction wherein luciferase converts NAD+ to NADH. Light emission from the luciferase reaction is proportional to the amount of NAD+ produced in the initial reaction. A candidate inhibitor compound is added to the ENR reaction, and if the candidate inhibits ENR activity, the amount of light detected in the luciferase reaction is decreased.

The assay was carried out as follows. Twenty μl of 30 μM NADH (Boehringer Manneheim) in 20 mM Hepes containing 6 ng/μl ENR or a total of 120 ng per well and 20 μl of 10 μM candidate inhibitor compound in DMSO were added to a 96 well flat bottom optical plate. Twenty μl of 300 μM crotonyl-CoA (Sigma, C6146) was subsequently added to initiate the reaction. Triclosan was used as a control inhibitor and was included on each plate to verify inhibition. In the screening assay, triclosan inhibits with an IC50 of about 1 μM.

The reaction was allowed to continue for approximately ten minutes, corresponding to about 30 percent of the way to completion. Accordingly, the concentration of NAD+ should be approximately 3 μM after ten minutes.

Thirty μl of 160 mM HCl was added to the system to bring the pH of the reaction mixture below 2 and remove remaining NADH substrate. The reaction mixture was incubated for one minute following acid addition so that the remaining NADH decomposes to ADP-ribose and nicotinamide. NAD+ is substantially unaffected by the addition of strong acid.

After the one minute period referenced immediately above, 110 μl of a NADH regeneration/luciferase solution comprising alcohol dehydrogenase, ethanol. FMN, FMN oxidoreductase, decanal and bacterial luciferase was added to the reaction mixture.

More specifically, the NADH regeneration/luciferase solution was prepared in 110 μl of a buffer solution containing 300 mM Tris (using a stock 1 M, pH 7.5 solution), 0.26% by weight bis(trimethylsilyl)acetamide (“BMA”), 0.65 mM EDTA, and 18 mM KCl. To this solution, 0.67 μl of decanal (Sigma, D7384, 98% purity) was added for every 10 ml of solution to yield a final solution having decanal concentration of approximately 200 μM. Sufficient FMN (Sigma F8399) was added to provide a final solution having a FMN concentration of approximately 2 μM. Similarly, sufficient ethanol (200 proof) was added such that the final solution has an ethanol concentration of approximately 100 μM. After adding all of these reagents to the solution, it was vortexed vigorously.

To this solution, 1.08 μl of NADH:FMN oxidoreductase (Roche, 476 480) was added for each 10 ml of solution, to yield a final solution having a concentration of 1.25 units per liter. Bacterial luciferase (Roche, 476 498) was added to yield a solution having approximately 4.5 μg/ml. Similarly, alcohol dehydrogenase was added to provide a solution having a final concentration of 0.7 units per ml. After adding these reagents, the mixture was mixed gently by inversion.

Approximately 100 compounds that inhibit ENR activity were identified in this assay. About 50 of these compounds exhibited significant inhibitory activity in a radiomimetric ENR assay. In this assay, twenty μl of 30 μM ³²P-NADH in 20 mM Hepes was incubated with 120 ng of ENR per well and 20 μl of 10 μM candidate inhibitor compound in DMSO. Twenty μl of 300 μM crotonyl-CoA (Sigma, C6146) was subsequently added to initiate the reaction. The products of the reaction include 32P-NAD. The reactant 32P-NADH and the product 32P-NAD are separated from each other by thin layer chromatography on PEI-cellulose in 1M LiCl, and visualized by autoradiography. The extent of the reaction is determined by the conversion of NADH to NAD. These compounds were further tested for inhibition of E. coli growth.

A permeable bacteria] strain (AB734 TN10::tolC) was used in the screening method to maximize the ability of the compounds to cross the gram negative cell wall. Assays were conducted in accordance with the NCCLS protocols referenced herein.

It was of further interest to determine whether those compounds with antimicrobial activity worked in a ENR-dependent fashion. Two strains of the permeable tolC strain were constructed. In the first strain, the ENR protein was overexpressed by placing it under control of its own promoter on a moderate copy number plasmid, and the second strain served as a control including only the plasmid. A candidate compound that targets ENR should be much less active against the first strain described above because the target is substantially overexpressed. For example, the MIC for triclosan shifts from 31 to 1000 ng/ml when tested against the first strain. Similarly, compound 325084 had a shift in MIC from 25 μM to greater than 100 μM, suggesting that this compound exerts its antimicrobial action by virtue of inhibiting ENR during bacterial growth. The results do not distinguish between the possibilities that compound 325084 and triclosan act on ENR at the same or distinct sites on the enzyme. However, because no other compound showed a similar shift in MIC, it is believed that these other compounds probably inhibit bacterial growth through a different mechanism. Nonetheless, compounds 325085 and 325086 have structures similar to compound 325084, and also demonstrated some activity against ENR.

In order to determine if compound 325084 and triclosan act at the same or different ENR sites, recombinant ENR is produced which (i) includes a glycine to valine substitution at residue 93, (ii) retains enzymatic activity, and (iii) is insensitive to triclosan. Compounds that are identified as ENR inhibitors are then assayed using both wild type and mutant ENR and compounds that show little or no inhibitory activity against the mutant ENR form are probably acting at the active site of ENR and may be discarded. Alternatively, compounds which inhibit the mutant enzyme and the wild type form may be acting at an allosteric site and will be studied further.

While the present invention has been described in terms of specific embodiments, it is understood that variations and modifications will occur to those skilled in the art. For example, with respect to the compounds disclosed herein, it should be understood that the substitution of one halogen substituent for another, different halogen substituent is within the scope of the present invention. Accordingly, only such limitations as appear in the appended claims should be placed on the present invention.

What is claimed is:

1. A method of modulating binding interaction between a first molecule which is not LFA-1 or an I domain-containing fragment thereof, and a binding partner molecule, said first molecule comprising an α/β domain structure, said α/β structure comprising an allosteric regulatory site, said method comprising the step of contacting said first molecule with an allosteric effector molecule that interacts with said allosteric regulatory site and promotes a conformation in a ligand binding domain of said α/β structure that modulates binding between said first molecule and said binding partner molecule.

2. A method of modulating binding interaction between a first molecule which is not LFA-1 or an I domain-containing fragment thereof, and a binding partner molecule, said first molecule comprising an α/β domain structure, said α/β structure comprising an allosteric regulatory site, said method comprising the step of contacting said first molecule with an allosteric effector molecule, said allosteric effector molecule comprising a diaryl compound, said diaryl compound interacting with said allosteric regulatory site and promoting a conformation in a ligand binding domain of said α/β structure that modulates binding between said first molecule and said binding partner molecule.

3. A method of modulating binding interaction between a first molecule which is not LFA-1 or an I domain-containing fragment thereof, and a binding partner molecule, said first molecule comprising an α/β domain structure, said α/β structure comprising an allosteric regulatory site, said method comprising the step of contacting said first molecule with an allosteric effector molecule, said allosteric effector molecule selected from the group consisting of diaryl sulfide compounds and diarylamide compounds, said allosteric effector molecule interacting with said allosteric regulatory site and promoting a conformation in a ligand binding domain of said α/β structure that modulates binding between said first molecule and said binding partner molecule.

4. The method of claim 1, 2, or 3 wherein said first molecule comprises a Rossmann fold structure, said Rossmann fold structure comprising said allosteric regulatory site.

5. The method of claim 4 wherein said Rossmann fold structure in said first molecule comprises a β sheet having β sheet strands positioned in a 321456 or 231456 orientation.

6. The method of claim 4 wherein said Rossmann fold structure in said first molecule comprises a β sheet having β sheet strands positioned in a 3214567 orientation.

7. The method of claim 4 wherein said Rossmann fold structure in said first molecule comprises a β sheet having β sheet strands positioned in a 32145 orientation.

8. The method of claim 1, 2, or 3 wherein said first molecule comprises an I domain structure.

9. The method of claim 1, 2, or 3 wherein said first molecule comprises an A domain structure.

10. A method of modulating binding interaction between a first molecule and a binding partner molecule, said first molecule having an amino acid sequence which exhibits less than about 90% identity to the LFA-1 I domain amino acid sequence set out in FIG. 1, said first molecule comprising an α/β structure, said α/β domain structure comprising an allosteric regulatory site, said method comprising the step of contacting said first molecule with an allosteric effector molecule that interacts with said allosteric regulatory site and promotes a conformation in a ligand binding domain of said α/β structure that modulates binding between said first molecule and said binding partner molecule.

11. A method of modulating binding interaction between a first molecule and a binding partner molecule, said first molecule having an amino acid sequence which exhibits less than about 90% identity to the LFA-1 I domain amino acid sequence set out in FIG. 1, said first molecule comprising an α/β structure, said α/β domain structure comprising an allosteric regulatory site, said method comprising the step of contacting said first molecule with an allosteric effector molecule, said allosteric effector molecule comprising a diaryl compound, said diaryl compound interacting with said allosteric regulatory site and promoting a conformation in a ligand binding domain of said α/β structure that modulates binding between said first molecule and said binding partner molecule.

12. A method of modulating binding interaction between a first molecule and a binding partner molecule, said first molecule having an amino acid sequence which exhibits less than about 90% identity to the LFA-1 I domain amino acid sequence set out in FIG. 1, said first molecule comprising an α/β domain structure, said α/β structure comprising an allosteric regulatory site, said method comprising the step of contacting said first molecule with an allosteric effector molecule, said allosteric effector molecule selected from the group consisting of diaryl sulfide compounds and diarylamide compounds, said allosteric effector molecule interacting with said allosteric regulatory site and promoting a conformation in a ligand binding domain of said α/β structure that modulates binding between said first molecule and said binding partner molecule.

13. The method of claim 10, 11, or 12 wherein said first molecule has an amino acid sequence that exhibits a percent identity with respect to the LFA-1 I domain amino acid sequence less than about 40%, about 45%, about 50%, about 55%, about 60%, about 65%, about 70%, about 75%, about 80%, about 85%, or about 90%.

14. The method of claim 10, 11, or 12 wherein said first molecule comprises a Rossmann fold structure, said Rossmann fold structure comprising an allosteric regulatory site.

15. The method of claim 14 wherein said first molecule has an amino acid sequence that exhibits a percent identity with respect to the LFA-1 I domain amino acid sequence less than about 40%, about 45%, about 50%, about 55%, about 60%, about 65%, about 70%, about 75%, about 80%, about 85%, or about 90%.

16. The method of claim 14 wherein said Rossmann fold structure in said first molecule comprises a β sheet having β sheet strands positioned in a 321456 or 231456 orientation.

17. The method of claim 16 wherein said first molecule has an amino acid sequence that exhibits a percent identity with respect to the LFA-1 I domain amino acid sequence less than about 40%, about 45%, about 50%, about 55%, about 60%, about 65%, about 70%, about 75%, about 80%, about 85%, or about 90%.

18. The method of claim 14 wherein said Rossmann fold structure in said first molecule comprises a β sheet having β sheet strands positioned in a 3214567 orientation.

19. The method of claim 18 wherein said first molecule has an amino acid sequence that exhibits a percent identity with respect to the LFA-1 I domain amino acid sequence less than about 40%, about 45%, about 50%, about 55%, about 60%, about 65%, about 70%, about 75%, about 80%, about 85%, or about 90%.

20. The method of claim 14 wherein said Rossmann fold structure in said first molecule comprises a β sheet having β sheets strands positioned in a 32145 orientation.

21. The method of claim 20 wherein said first molecule has an amino acid sequence that exhibits a percent identity with respect to the LFA-1 I domain amino acid sequence less than about 40%, about 45%, about 50%, about 55%, about 60%, about 65%, about 70%, about 75%, about 80%, 85%, or about 90%.

22. The method of claim 10, 11, or 12 wherein said first molecule comprises an I domain structure.

23. The method of claim 23 wherein said first molecule has an amino acid sequence that exhibits a percent identity with respect to the LFA-1 I domain amino acid sequence less than about 40%, about 45%, about 50%, about 55%, about 60%, about 65%, about 70%, about 75%, about 80%, about 85%,or about 90%.

24. The method of claim 11, 11, or 12 wherein said first molecule comprises an A domain structure.

25. The method of claim 24 wherein said first molecule has an amino acid sequence that exhibits a percent identity with respect to the LFA-1 I domain amino acid sequence less than about 40%, about 45%, about 50%, about 55%, about 60%, about 65%, about 70%, about 75%, about 80%, about 85%, or about 90%.

26. The method of any one of claims 1-3, 5-7, 10-12, 15-21, 23 or 25 wherein the modulator promotes a conformation in the ligand binding domain of said first molecule that increases binding between said first molecule and said binding partner molecule.

27. The method of claim 26 wherein the increase in binding between the first molecule and the second molecule results in increased enzymatic activity of the first molecule.

28. The method of any one of claims 1-3, 5-7, 10-12, 15-21, 23 or 25 wherein the modulator promotes a conformation in the ligand binding domain of said first molecule that decreases binding between said first molecule and said binding partner molecule.

29. The method of claim 28 wherein the decrease in binding between the first molecule and the second molecule results in decreased enzymatic activity of the first molecule.

30. The method of any one of claims 1-3, 5-7, 10-12, 15-21, 23 or 25 wherein the first molecule is selected from the group consisting of the proteins set forth in Table 1.

31. The method of claim 30 wherein the first molecule is a eukaryotic molecule.

32. The method of claim 30 wherein the first molecule is a human molecule.

33. The method of claim 30 wherein the first molecule is a prokaryotic molecule.

34. The method of claim 30 wherein the first molecule is a bacterial molecule.

35. The method of claim 30 wherein the first molecule is selected from the group consisting of α_{M}β₂, complement protein C2, complement protein Factor B, α_{E}β₇, α₄β₇, α_{V}β₃, α₄β₁, α_{d}β₂, von Willebrand factor, Rac-1, HPPK, ftsZ, and ENR.

36. The method of claim 35 wherein the first molecule is α_{M}β₂ and the binding partner protein is fibrinogen.

37. The method of claim 35 wherein the first molecule is α_{M}β₂ and the binding partner protein is iC3b.

38. The method of claim 35 wherein the first molecule is α_{E}β₇ and the binding partner protein is E-cadherin.

39. The method of claim 35 wherein the first molecule is α₄β₇ and the binding partner protein is MAdCAM-1.

40. The method of claim 35 wherein the first molecule is α_{V}β₃ and the binding partner protein is vitronectin.

41. The method of claim 35 wherein the first molecule is α₄β₁ and the binding partner protein is VCAM.

42. The method of claim 35 wherein the first molecule is α_{d}β₂ and the binding partner protein is VCAM.

43. The method of claim 35 wherein the first molecule is von Willebrand factor and the binding partner protein is gpIb.

44. The method of claim 35 wherein the first molecule is complement protein C2 and the binding partner protein is complement protein C4b.

45. The method of claim 35 wherein the first molecule is complement protein Factor B and the binding partner protein is complement protein C3b.

46. The method of claim 35 wherein the first molecule is Rac-1 and the binding partner is GTP.

47. The method of claim 35 wherein the first molecule is HPPK and the binding partner is ATP or HMDP.

48. The method of claim 35 wherein the first molecule is ftsZ and the binding partner is GTP.

49. The method of claim 35 wherein the first molecule is ENR and the binding partner is NADH.
